(19) United States
(12) Patent Application Publication
(10) Pub. No.: US 2024/0165153 A1
(43) Pub. Date: May 23, 2024

(54) BICISTRONIC CHIMERIC ANTIGEN RECEPTORS DESIGNED TO REDUCE RETROVIRAL RECOMBINATION AND USES THEREOF

(71) Applicant: The United States of America,as represented by the Secretary,Department of Health and Human Services, Bethesda, MD (US)

(72) Inventors: James N. Kochenderfer, Bethesda, MD (US); Norris Lam, Silver Spring, MD (US)

(73) Assignee: The United States of America,as represented by the Secretary,Department of Health and Human Services, Bethesda, MD (US)

(21) Appl. No.: 18/282,919

(22) PCT Filed: Mar. 23, 2022
(86) PCT No.: PCT/US2022/021545

§ 371 Date: Sep. 19, 2023

Related U.S. Application Data
(60) Provisional application No. 63/165,195, filed on Mar. 24, 2021.

(51) Int. Cl. A61K 35/17; A61K 39/00; C07K 16/28; C12N 15/86

(52) U.S. Cl. CPC A61K 35/17; A61K 39/4611; C07K 16/2803; C07K 16/2887; C12N 15/86; A61K 2039/507; A61K 2039/5156; A61K 2239/13; C07K 2317/53; C07K 2319/03; C12N 2740/13043

(57) ABSTRACT

An aspect of the invention provides nucleic acids comprising a nucleotide sequence encoding chimeric antigen receptor (CAR) amino acid constructs. Polypeptides, recombinant expression vectors, host cells, populations of cells, and pharmaceutical compositions relating to the CAR constructs are disclosed. Methods of detecting the presence of cancer in a mammal and methods of treating or preventing cancer in a mammal are also disclosed.

### CROSS-REFERENCE TO RELATED APPLICATION

This patent application claims the benefit of U.S. Provisional Patent Application No. 63/165,195 filed Mar. 24, 2021, which is incorporated by reference herein in its entirety.

### STATEMENT REGARDING FEDERALLY SPONSORED RESEARCH AND DEVELOPMENT

This invention was made with Government support under project number ZIA BC011417 09 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 127,516 byte ASCII (text) file named “759440_ST25.txt” dated Feb. 7, 2022.

### BACKGROUND

Cancer is a public health concern. Despite advances in treatments such as chemotherapy, the prognosis for many cancers, including hematological malignancies, may be poor. Accordingly, there exists an unmet need for additional treatments for cancer, particularly hematological malignancies.

### BRIEF SUMMARY

In aspects, the disclosure provides a nucleic acid comprising a nucleotide sequence encoding a chimeric antigen receptor (CAR) construct comprising: (a) a first CAR comprising a first antigen binding domain, a first transmembrane domain, and a first intracellular T cell signaling domain; (b) a second CAR comprising a second antigen binding domain, a second transmembrane domain, and a second intracellular T cell signaling domain; and (c) a cleavage sequence; wherein the cleavage sequence is positioned between the first and second CARs, and wherein the nucleic acid has been designed to reduce retroviral recombination.

In aspects, the disclosure provides a method of making a chimeric antigen receptor (CAR) construct, the method comprising: (i) designing a nucleic acid comprising a nucleotide sequence encoding the CAR construct comprising (a) a first CAR comprising a first antigen binding domain, a first transmembrane domain, and a first intracellular T cell signaling domain;(b) a second CAR comprising a second antigen binding domain, a second transmembrane domain, and a second intracellular T cell signaling domain; and (c) a cleavage sequence; wherein the cleavage sequence is positioned between the first and second CARs; (ii) designing the nucleic acid to reduce retroviral recombination; and (iii) preparing the nucleic acid of (ii).

In aspects, the disclosure provides a nucleic acid comprising a nucleotide sequence encoding a CAR comprising the nucleic acid sequence of any one of SEQ ID NOS: 42-45 and 48-52.

Further aspects of the disclosure provide related polypeptides encoded by the nucleic acids, recombinant expression vectors, host cells, populations of cells, and pharmaceutical compositions.

Additional aspects of the disclosure provide related methods of detecting the presence of and treating or preventing cancer in a mammal.

Additional aspects are as described herein.

### BRIEF DESCRIPTION OF THE DRAWINGS

FIG. 1 presents a diagram of a bicistronic CAR construct and, without wishing to be bound by theory, a possible mechanism of deletion of a sequence driven by regions of sequence similarity. The diagram depicts a mechanism of intramolecular deletion driven by base pairing of nascent proviral DNA to two identical repeated regions of sequence in the retroviral genomic RNA during reverse transcription. Step 1: Genomic RNA with two identical repeated regions of sequence (Sequence 1 and Sequence 2). Step 2: Reverse transcription begins at the 3′ end of the genomic RNA. Step 3: If two regions of identical RNA sequence come into close proximity, simultaneous base pairing can occur between both of the identical repeated RNA sequences and the nascent DNA strand. RNAse H digests RNA after it has been reverse transcribed. Step 4: Intramolecular template switch can occur, so that reverse transcription continues at the 5′ region of identical sequence (Sequence 1) rather than the 3′ region of identical sequence (Sequence 2) where reverse transcription was occurring prior to the template switch. Step 5: This results in one copy of the identical regions of sequence being incorporated into the final DNA provirus with the sequence between the identical regions of sequence deleted.

FIG. 2 presents alignment of the first (SEQ ID NO: 1) and second (SEQ ID NO: 2) CD8α hinge and transmembrane domains of the Hu1928-Hu20BB-original CAR. Nucleotides that are different between the two regions are capitalized. The numbers on the right side of the figure are the locations of the CD8α hinge and transmembrane region nucleotides covered by each alignment. The two areas of underlined nucleotides indicate the 8 nucleotides immediately 5′ to areas of deleted sequence that occurred in a minority of RNA transcripts when the Hu1928-Hu20BB-original construct was used to transduce human T cells. These deleted regions are consistent with recombination events driven by areas of identical nucleotide sequences in different parts of the CAR construct.

FIG. 3 presents diagrams of bicistronic CAR constructs.

FIG. 4 presents diagrams of monospecific CARs.

FIG. 5 presents an alignment of the first (SEQ ID NO: 3) and second (SEQ ID NO: 4) CD8α hinge and transmembrane domains of Hu1928-Hu20BB std 10-5-2020. Nucleotides that are different between the two regions are capitalized. The numbers on the right side of the figure are the locations of the CD8α hinge and transmembrane region nucleotides covered by each alignment. Changes were made in nucleotides to decrease series of identical nucleotides in order to reduce the change of recombination events in Hu1928-Hu20BB std 10-5-2020 versus Hu1928-Hu20BB original.

FIGS. 6A-6D present dot plots showing CAR expression on the surface of transduced T cells. FIG. 6A shows CD4⁺ cells stained with anti-Hu20. FIG. 6B shows CD4+ cells stained with anti-Hu19. FIG. 6C shows CD8⁺ T cells stained with anti-Hu20. FIG. 6D shows CD8⁺ T cells stained with anti-Hu19. Figures show one of four representative experiments with similar results.

FIGS. 7A-7C present dot plots showing CD4⁺ T cells transduced with bicistronic CAR constructs degranulate specifically in response to CD19 and CD20. FIG. 7A presents plots for T cells transduced with Hu1928-Hu20BB std 10-5-2020; FIG. 7B presents plots for T cells transduced with Hu1928-Hu20BB long 10-21-2020; and FIG. 7C presents plots for untransduced T cells. Plots show cells gated on CD3⁺, CD4⁺ live lymphocytes. CD107a+ events indicate degranulation. These present one of four experiments having similar results.

FIGS. 8A-8C present dot plots showing CD8⁺ T cells transduced with bicistronic CAR constructs degranulate specifically in response to CD19 and CD20. FIG. 8A presents plots for T cells transduced with Hu1928-Hu20BB std 10-5-2020; FIG. 8B presents plots for T cells transduced with Hu1928-Hu20BB long 10-21-2020; and FIG. 8C presents plots for untransduced T cells. Plots show cells gated on CD3⁺, CD8⁺ live lymphocytes. CD107a⁺ events indicate degranulation. These present one of four experiments having similar results.

FIGS. 9A and 9B present graphs showing CD4⁺ T cells transduced with bicistronic CAR constructs proliferated in an antigen-specific manner. Histograms are gated on CD3⁺, CD4⁺, CAR⁺, live lymphocytes and show the CFSE fluorescence for T cells cultured with the indicated target cells. CD19-K562 or CD20-K562 antigen-expressing target cells are the larger peaks in each graph, and antigen-negative NGFR-K562 cells are the smaller peaks in each graph. Lower CFSE fluorescence indicates more proliferation of the transduced T cells. FIG. 9A shows results for T cells expressing the Hu1928-Hu20BB std 10-5-2020 CAR construct, and FIG. 9B shows results for T cells expressing the Hu1928-Hu20BB long 10-21-2020 CAR construct. These present one of two experiments having similar results.

FIGS. 10A and 10B present graphs showing CD8⁺ T cells transduced with bicistronic CAR constructs proliferated in an antigen-specific manner. Histograms are gated on CD3⁺, CD8⁺, CAR⁺, live lymphocytes and show the CFSE fluorescence for T cells cultured with the indicated target cells. CD19-K562 or CD20-K562 antigen-expressing target cells are the larger peaks in each graph, and antigen-negative NGFR-K562 cells are the smaller peaks in each graph. Lower CFSE fluorescence indicates more proliferation of the transduced T cells. FIG. 10A shows results for T cells expressing the Hu1928-Hu20BB std 10-5-2020 CAR construct, and FIG. 10B shows results for T cells expressing the Hu1928-Hu20BB long 10-21-2020 CAR construct. These present one of two experiments having similar results.

FIGS. 11A-11D show dot plots showing expression of Hu1928-Hu2028 long and HU1928-Hu20BB long 10-21-2020. All plots are gated on live, CD3⁺ lymphocytes. FIG. 11A presents plots for CD4⁺ cells stained with anti-Hu20. FIG. 11B presents plots for CD4⁺ cells stained with anti-Hu19. FIG. 11C presents plots for CD8⁺ T cells stained with anti-Hu20. FIG. 11D presents plots for CD8⁺ T cells stained with anti-Hu19. These present one of three experiments having similar results.

FIGS. 12A-12N present dot plots and line graphs showing expression of CARs described herein. FIG. 12A shows human PBMC stimulated with anti-CD3 in IL-2-containing media, with transductions conducted two days later. Five days later, flow cytometry was conducted to assess CAR expression. Plots gated on live CD3⁺ lymphocytes show combined expression of anti-CD19 and anti-CD20 CARs on T cells transduced with Hu1928-Hu20BB-Original (Original) or Hu1928-Hu20BB std 10-5-2020 (STD) constructs. FIG. 12B shows a summary of 4 experiments conducted as in FIG. 12A with cells from 4 donors. Statistical comparison was by two-tailed, paired t test. FIGS. 12C-12F present plots showing expression of anti-CD19 and anti-CD20 CARs on CD4⁺ (FIGS. 12C and 12D) and CD8⁺ (FIGS. 12E and 12F) T cells transduced with STD or Hu1928-Hu20BB long 10-21-2020 (LONG) constructs. Untransduced T cells are also shown. For FIGS. 12C-12F, plots are gated on live CD3+ lymphocytes. FIGS. 12G and 12H show T cells cultured and transduced, with flow cytometry performed as in FIGS. 12C and 12D. Percentages of CD4⁺ (FIG. 12G) and CD8⁺ (FIG. 12H) T cells staining with anti-CD19 CAR antibody were compared for STD and LONG. FIGS. 12I and 12J show T cells were cultured and transduced, with flow cytometry performed as in FIGS. 12E and 12F. Percentages of CD4⁺ (FIG. 12I) and CD8⁺ (FIG. 12J) T cells staining with the anti-CD20 CAR antibody were compared for STD and LONG. FIGS. 12K and 12L show T cells expressing STD or LONG cultured for 4 hours with st486 cells in the presence of an antibody against CD107a. Cells were assessed by flow cytometry for CD107a expression on live CD3⁺ CD4⁺ T cells (FIG. 12K) and live CD3⁺ CD8⁺ T cells (FIG. 12L).

FIGS. 12M and 12N show cells assessed for CD107a expression in the same manner as FIGS. 12K and 12L except st486-CD19neg target cells were used. For FIGS. 12G-12N, statistical comparison was by 2-tailed paired t test; n=4 and N.S. means not significant.

FIG. 13A presents a bar graph showing T cells transduced with the indicated CAR constructs or left untransduced and cultured overnight with target cells. An IFNg ELISA was performed on culture supernatants; n=4 different donors. CD19-K562 target cells expressed CD19. CD20-K562, st486-CD19neg, and Toledo-CD19neg expressed CD20. St486 and Toledo expressed CD19 and CD20. CCRF-CEM and NGFR-K562 were negative for CD19 and CD20. Statistics were two-tailed, paired ratio t tests; *indicates P<0.05, ** indicates P<0.001; cytokine values were normalized for CAR expression by dividing cytokine values by the fraction of T cells expressing both CARs in the constructs.

FIG. 13B presents a bar graph showing supernatants from the same CAR T cell plus target cell cultures as in FIG. 13A assessed by ELISA for IL-2; n=5 donors except n=4 for Toledo and Toledo-CD19neg. Statistics were two-tailed, paired ratio t tests; *indicates P<0.05, ** indicates P<0.001; cytokine values were normalized for CAR expression by dividing cytokine values by the fraction of T cells expressing both CARs in the constructs.

FIGS. 13C and 13D presents dot plots showing T cells expressing either Hu1928-Hu20BB std 10-5-2020 (STD) (FIG. 13C) or Hu1928-Hu20BB long 10-21-2020 (LONG) (FIG. 13D) cultured for 4 hours with either st486-CD19neg or NGFR-K562 target cells. CD4⁺ and CD8⁺ T cells were then assessed for anti-CD20 CAR expression. Plots are gated on live (7aad-negative), CD3⁺ lymphocytes.

FIGS. 13E and 13F present dot plots showing annexin V staining of the same cells from FIGS. 13C and 13D, respectively. Plots are gated on live CAR+ CAR⁺ CD4⁺ or CAR⁺ CD8⁺ T cells.

FIG. 13G presents a line graph showing cells analyzed by flow cytometry as shown in FIGS. 13C and 13D. The decrease in antigen-specific CAR expression of Hu20-CARs was quantified as the percent anti-CD20 CAR⁺ cells with st486-CD19neg stimulation divided by the percent anti-CD20 CAR⁺ cells with NGFR-K562 stimulation (% CAR⁺ with st486-CD19-neg/NGFR-K562 stim). Results are for CD4⁺ cells. n=6, and statistics are two-tailed paired t tests.

FIG. 13H presents a line graph showing the same analysis as in FIG. 13G on cells from the same cultures as in FIG. 13G shown for CD8⁺ cells. n=6, and statistics are two-tailed paired t tests.

FIG. 13I presents a line graph showing the percentage of specific annexin V expression for the same CD4⁺ CAR⁺ T cells analyzed in FIG. 13G. Specific % Annexin V⁺ cells were calculated as the % Annexin V⁺ CAR⁺ T cells with st486-CD19neg stimulation minus the % Annexin V⁺ CAR⁺ T cells with NGFR-K562 stimulation. n=6, and statistics are two-tailed paired t tests.

FIG. 13J presents a line graph showing the percentage of specific annexin V expression for the same CD8⁺ CAR⁺ T cells analyzed in FIG. 13H. Specific % Annexin V⁺ cells were calculated as the % Annexin V⁺ CAR⁺ T cells with st486-CD19neg stimulation minus the % Annexin V⁺ CAR⁺ T cells with NGFR-K562 stimulation. n=6, and statistics are two-tailed paired t tests.

FIG. 14A is a line graph showing T cells expressing Hu1928-Hu20BB std 10-5-2020 (STD), Hu1928-Hu20BB long 10-21-2020 (LONG), or the negative-control CAR SP6-CD828Z incubated with Toledo cells for 4 hours, with cytoxicity assessed. This is one of 2 experiments with nearly identical results.

FIG. 14B is a line graph. Four million st486 cells were injected intradermally into NSG mice, and six days later when palpable tumors were present, mice were injected intravenously with a single infusion of 1×10⁶ CAR⁺ T cells or left untreated as indicated. Ten total mice from 2 separate experiments of 5 mice each are in each group. Each experiment used T cells from a different human donor. Values are mean tumor volume+/− SEM.

FIG. 14C is a Kaplan-Meier plot of survival of the same mice as in FIG. 14B. Survival was statistically longer when the Hu1928-Hu20BB std 10-5-2020 (STD) or Hu1928-Hu20BB long 10-21-2020 (LONG) groups were compared to the SP6-CD828Z group (P<0.0001). Survival was statistically longer when the STD or LONG groups were compared to the Untreated group (P<0.0001). There was no statistically-significant difference in survival between STD and LONG. Survival comparison by Log-rank test.

FIG. 15A presents dot plots showing 10-5-2020 Hul9-CD828Z and Hu20-CD8BBZ long expression on T cells transduced with the indicated CAR constructs assessed by flow cytometry five days after transduction. Transductions were performed as described for FIG. 12. Plots are gated on live CD3⁺ T cells. Similar results were obtained with cells from 9 donors.

FIG. 15B is a bar graph showing T cells transduced with the indicated CAR constructs or left untransduced cultured overnight with the indicated target cells, with an IFNγ ELISA performed on culture supernatants. With CD19⁺ CD19-K562 target cells, Hu20-CD8BBZ long T cells had statistically lower IFNγ production compared with 10-5-2020 Hul9-CD828Z T cells and Hu1928-Hu20BB long 10-21-2020 (LONG) T cells. 10-5-2020 Hul9-CD828Z T cells had statistically lower IFNγ production than T cells expressing Hu20-CD8BBZ long or LONG when T cell were stimulated with the CD19-negative target cells CD20-K562 and st486-CD19neg or st486 target cells that weakly express CD19. Compared with T cells expressing the other two CAR constructs, Hu20-CD8BBZ long T cells had higher non-specific IFNγ release against negative control target cells. CAR T-cell types with statistically different levels of IFNγ release than the other 2 CAR T-cell types at the P<0.01 by level two-tailed, paired ratio t test are indicated by*. Bars represent mean+SEM; n=5 donors. Values are pg/mL of IFNγ normalized for CAR expression.

FIG. 15C is a line graph. NSG mice were injected with CD20⁺, CD19-negative CD20-MM.1S cells. Seven days later, when palpable tumors were present, mice received intravenous injections of 3×10⁶ CAR⁺ human T cells of the indicated types or were left untreated. Values are mean tumor volume+/−SEM; n=5 mice per group.

FIG. 15D is a Kaplan-Meier survival plot of the same mice as in FIG. 15C. By log-rank test, survival was longer for Hu20-CD8BBZ long and Hu1928-Hu20BB long 10-21-2020 (LONG) versus the other 3 groups; P<0.003.

FIG. 15E is a line graph. NSG mice were injected with NALM6 cells. When palpable tumors were present 6 days later, mice received the indicated number of Hu1928-Hu20BB long 10-21-2020 (LONG) CAR⁺ T cells or were left untreated. Values are mean tumor volume+/−SEM; n=5 mice per group.

FIG. 15F is a Kaplan-Meier survival plot of mice from FIG. 15E. Survival of all LONG groups was longer than survival of untreated mice; P<0.005 by log-rank test.

### DETAILED DESCRIPTION

Recombination events driven by binding of identical regions of nucleotides can occur during the production and use of retroviral vectors. Recombination between homologous nucleotide sequences can occur during production of retroviral vectors in packaging cells. Recombination events leading to deletions of intended sequences can also occur during reverse transcription after retroviruses infect target cells. Recombination events during reverse transcription occur when areas of identical nucleotide sequence on the same nucleotide strand anneal, which leads to strand switching by the reverse transcriptase enzyme and deletion of some of the intended sequence.

In aspects, the disclosure provides a nucleic acid comprising a nucleotide sequence encoding a chimeric antigen receptor (CAR) construct comprising: (a) a first CAR comprising a first antigen binding domain, a first transmembrane domain, and a first intracellular T cell signaling domain; (b) a second CAR comprising a second antigen binding domain, a second transmembrane domain, and a second intracellular T cell signaling domain; and (c) a cleavage sequence; wherein the cleavage sequence is positioned between the first and second CARs, and wherein the nucleic acid has been designed to reduce retroviral recombination.

A CAR is an artificially constructed hybrid protein or polypeptide containing an antigen binding domain of an antibody linked to T-cell signaling or T-cell activation domains. CARs have the ability to redirect T-cell specificity and reactivity toward a selected target in a non-MHC-restricted manner, exploiting the antigen-binding properties of monoclonal antibodies. The non-MHC-restricted antigen binding gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

In aspects, the nucleic acid sequence identity between the first and second CARs is no more than 99%, 98%, 97%, 96%, 95%, 94%, 93%, 92%, 91%, 90%, 89%, 88%, 87%, 86%, 85%, 84%, 83%, 82%, 81%, or 80%. In aspects, the nucleic acid sequence identity between the first and second CARs is no more than 90%. In aspects, the nucleotide sequence of the CAR construct has been designed to reduce areas where a series of 8, 9, 10, 11, 12, 13, 14, 15, 20, or 25 or more contiguous nucleotides occur more than once within the CAR construct. In aspects, the CAR construct has been designed by changing the linker length in a single-chain variable domain (scFv) and by use of either CD28 or 4-1BB costimulatory domains. In aspects, the nucleic acid, when expressed in a host cell, exhibits greater expression compared to a nucleic acid that encodes the same amino acid sequence but that has not been designed to reduce retroviral recombination.

In aspects, the first antigen binding domain of the first CAR has antigenic specificity for CD19, and the second antigen binding domain of the second CAR has antigenic specificity for CD20. The phrases “has antigenic specificity” and “elicit antigen-specific response,” as used herein, means that the CAR can specifically bind to and immunologically recognize an antigen, such that binding of the CAR to the antigen elicits an immune response.

CD19 (also known as B-lymphocyte antigen CD19, B4, and CVID3) is a cell surface molecule expressed only by B lymphocytes and follicular dendritic cells of the hematopoietic system. It is the earliest of the B-lineage-restricted antigens to be expressed and is present on most pre-B-cells and most non-T-cell acute lymphocytic leukemia cells and B-cell type chronic lymphocytic leukemia cells.

CD20 (also known as B-lymphocyte antigen CD20) is an activated-glycosylated phosphoprotein expressed on the surface of all B-cells. CD20 is found on B-cell lymphomas, hairy cell leukemia, B-cell chronic lymphocytic leukemia, transformed mycosis fungoides, and melanoma cancer stem cells.

In aspects, the nucleic acid sequence may comprise, consist of, or consist essentially of the nucleotide sequence of any one of SEQ ID NO: 42 (Hu1928-Hu20BB standard (std) 10-5-2020), SEQ ID NO: 43 (Hu1928-Hu20BB long 10-21-2020), SEQ ID NO: 44 (Hu1928-Hu2028 long), or SEQ ID NO: 45 (Hu1928-Hu2028 std (standard)). In aspects, the nucleic acid sequence may comprise, consist of, or consist essentially of the nucleotide sequence of any one of SEQ ID NO: 48 (10-5-2020 Hul9-CD828Z), SEQ ID NO: 49 (9-15-2020 Hu20-CD8BBZ std), SEQ ID NO: 50 (Hu20-CD828Z std), SEQ ID NO: 51 (Hu20-CD8BBZ long), or SEQ ID NO: 52 (Hu20-CD828Z long).

The inventive bicistronic CAR constructs may provide any one or more of a variety of advantages. Although CAR T cells have been known to be a successful therapy, loss of antigen expression after CAR T-cell therapy has been found to be a mechanism for failure of this treatment approach (e.g., loss of CD19 expression has been detected in acute lymphoid leukemia and B-cell lymphomas). The inventive bicistronic CAR constructs may allow treatment of malignancies that lose expression of one antigen, e.g., CD19 or CD20, if expression of one of the two antigens is retained. By targeting two antigens, e.g., CD19 and CD20, the inventive CAR constructs advantageously provide an alternative strategy for treating cancer. Further, the inventive nucleic acids require only one gene therapy vector to engineer a patient's T cells to express two CARs. A single T cell can simultaneously express both CARs.

The first CAR comprises a first antigen binding domain. In aspects, the first antigen binding domain recognizes and binds to CD19. The antigen binding domain of the CAR may comprise the antigen binding domain of an anti-CD19 antibody.

The second CAR comprises a second antigen binding domain. In aspects, the second antigen binding domain recognizes and binds to CD20. The antigen binding domain of the CAR may comprise the antigen binding domain of an anti-CD20 antibody.

In aspects, the first and second antigen binding domains may comprise any antigen binding portion of an antibody. For example, the antigen binding domain may be a Fab fragment (Fab), F(ab')2 fragment, diabody, triabody, tetrabody, single-chain variable region fragment (scFv), or a disulfide-stabilized variable region fragment (dsFv). In a preferred aspect, the antigen binding domain is an scFv. An scFv is a truncated Fab fragment including the variable (V) domain of an antibody heavy chain linked to a V domain of an antibody light chain via a synthetic peptide, which can be generated using routine recombinant DNA technology techniques. The antigen binding domains employed in the inventive CARs, however, are not limited to these exemplary types of antibody fragments.

In aspects, the first antigen binding domain may comprise a light chain variable region and/or a heavy chain variable region of an anti-CD19 antibody. In aspects of the disclosure, the heavy chain variable region of the first antigen binding domain comprises a heavy chain complementarity determining region (CDR) 1, a heavy chain CDR2, and a heavy chain CDR3 of an anti-CD19 antibody. In aspects of the disclosure, the light chain variable region of the first antigen binding domain may comprise a light chain CDR1, a light chain CDR2, and a light chain CDR3 of an anti-CD19 antibody. In a preferred aspect, the first antigen binding domain comprises all of a heavy chain CDR1, a heavy chain CDR2, a heavy chain CDR3, a light chain CDR1, a light chain CDR2, and a light chain CDR3 of an anti-CD19 antibody.

In aspects, the second antigen binding domain may comprise a light chain variable region and/or a heavy chain variable region of an anti-CD20 antibody. In aspects of the disclosure, the heavy chain variable region of the second antigen binding domain comprises a heavy chain CDR1, a heavy chain CDR2, and a heavy chain CDR3 of an anti-CD20 antibody. In aspects of the disclosure, the light chain variable region of the second antigen binding domain may comprise a light chain CDR1, a light chain CDR2, and a light chain CDR3 of an anti-CD20 antibody. In a preferred aspect, the second antigen binding domain comprises all of a light chain CDR1, a light chain CDR2, a light chain CDR3, a heavy chain CDR1, a heavy chain CDR2, and a heavy chain CDR3 of an anti-CD20 antibody.

In aspects of the disclosure, the first antigen binding domain of the CAR is the antigen binding domain of the scFv Hul9. The antigen binding domain of Hul9 specifically binds to CD19. The Hul9 scFv is described in Alabanza et al., Molecular Ther., 25: 2452-2465 (2017). The inventive first CAR may comprise all of the light chain CDR1, the light chain CDR2, the light chain CDR3, the heavy chain CDR1, the heavy chain CDR2, and the heavy chain CDR3 of Hul9.

In aspects of the disclosure, the first antigen binding domain of the CAR is the antigen binding domain of 47G4. The antigen binding domain of 47G4 specifically binds to CD19. The inventive first CAR may comprise all of the light chain CDR1, the light chain CDR2, the light chain CDR3, the heavy chain CDR1, the heavy chain CDR2, and the heavy chain CDR3 of 47G4.

In aspects of the disclosure, the second antigen binding domain of the CAR is the antigen binding domain of the antibody C2B8. The antigen binding domain of C2B8 specifically binds to CD20. The C2B8 antibody is described in U.S. Pat. No. 5,736,137, incorporated by reference herein in its entirety. The inventive second CAR may comprise all of the light chain CDR1, the light chain CDR2, the light chain CDR3, the heavy chain CDR1, the heavy chain CDR2, and the heavy chain CDR3 of C2B8.

In aspects of the disclosure, the second antigen binding domain of the CAR is the antigen binding domain of the antibody 11B8. The antigen binding domain of 11B8 specifically binds to CD20. The 11B8 antibody is described in U.S. Patent Application 2004/0167319, incorporated by reference herein in its entirety. The inventive second CAR may comprise all of the light chain CDR1, the light chain CDR2, the light chain CDR3, the heavy chain CDR1, the heavy chain CDR2, and the heavy chain CDR3 of 11B8.

In aspects of the disclosure, the second antigen binding domain of the CAR is the antigen binding domain of the antibody 8G6-5. The antigen binding domain of 8G6-5 specifically binds to CD20. The 8G6-5 antibody is described in U.S. Patent Application 2009/0035322, incorporated, by reference herein in its entirety. The inventive second CAR may comprise all of the light chain CDR1, the light chain CDR2, the light chain CDR3, the heavy chain CDR1, the heavy chain CDR2, and the heavy chain CDR3 of the antibody 8G6-5.

In aspects of the disclosure, the second antigen binding domain of the CAR is the antigen binding domain of the antibody 2.1.2. The antigen binding domain of 2.1.2 specifically binds to CD20. The 2.1.2 antibody is described in WO 2006/130458, incorporated by reference herein in its entirety. The inventive second CAR may comprise all of the light chain CDR1, the light chain CDR2, the light chain CDR3, the heavy chain CDR1, the heavy chain CDR2, and the heavy chain CDR3 of the antibody 2.1.2.

In aspects of the disclosure, the second antigen binding domain of the CAR is the antigen binding domain of the antibody GA101. The antigen binding domain of GA101 specifically binds to CD20. The GA101 antibody is described in U.S. Pat. No. 9,539,251, incorporated by reference herein in its entirety. The inventive second CAR may comprise all of the light chain CDR1, the light chain CDR2, the light chain CDR3, the heavy chain CDR1, the heavy chain CDR2, and the heavy chain CDR3 of the antibody GA101.

In aspects of the disclosure, the Hul9 antigen binding domain comprises a heavy chain variable region and a light chain variable region. The heavy chain variable region of the Hul9 antigen binding domain may comprise, consist of, or consist essentially of the amino acid sequence of SEQ ID NO: 5. The light chain variable region of the Hul9 antigen binding domain may comprise, consist of, or consist essentially of the amino acid sequence of SEQ ID NO: 6. Accordingly, in aspects of the disclosure, the Hul9 antigen binding domain comprises a heavy chain variable region comprising the amino acid sequence of SEQ ID NO: 5 and/or a light chain variable region comprising the amino acid sequence of SEQ ID NO: 6. Preferably, the Hul9 antigen binding domain comprises the amino acid sequences of both SEQ ID NOs: 5 and 6.

In aspects of the disclosure, the C2B8 antigen binding domain comprises a heavy chain variable region and a light chain variable region. The heavy chain variable region of the C2B8 antigen binding domain may comprise, consist of, or consist essentially of the amino acid sequence of SEQ ID NO: 7. The light chain variable region of the C2B8 antigen binding domain may comprise, consist of, or consist essentially of the amino acid sequence of SEQ ID NO: 8. Accordingly, in aspects of the disclosure, the C2B8 antigen binding domain comprises a heavy chain variable region comprising the amino acid sequence of SEQ ID NO: 7 and/or a light chain variable region comprising the amino acid sequence of SEQ ID NO: 8. Preferably, the C2B8 antigen binding domain comprises the amino acid sequences of both SEQ ID NOs: 7 and 8.

In aspects of the disclosure, the 11B8 antigen binding domain comprises a heavy chain variable region and a light chain variable region. The heavy chain variable region of the 111B8 antigen binding domain may comprise, consist of, or consist essentially of the amino acid sequence of SEQ ID NO: 9. The light chain variable region of the 11B8 antigen binding domain may comprise, consist of, or consist essentially of the amino acid sequence of SEQ ID NO: 10. Accordingly, in aspects of the disclosure, the 11B8 antigen binding domain comprises a heavy chain variable region comprising the amino acid sequence of SEQ ID NO: 9 and/or a light chain variable region comprising the amino acid sequence of SEQ ID NO: 10. Preferably, the 11B8 antigen binding domain comprises the amino acid sequences of both SEQ ID NOs: 9 and 10.

In aspects of the disclosure, the 8G6-5 antigen binding domain comprises a heavy chain variable region and a light chain variable region. The heavy chain variable region of the 8G6-5 antigen binding domain may comprise, consist of, or consist essentially of the amino acid sequence of SEQ ID NO: 11. The light chain variable region of the 8G6-5 antigen binding domain may comprise, consist of, or consist essentially of the amino acid sequence of SEQ ID NO: 12. Accordingly, in aspects of the disclosure, the 8G6-5 antigen binding domain comprises a heavy chain variable region comprising the amino acid sequence of SEQ ID NO: 11 and/or a light chain variable region comprising the amino acid sequence of SEQ ID NO: 12. Preferably, the 8G6-5 antigen binding domain comprises the amino acid sequences of both SEQ ID NOs: 11 and 12.

In aspects of the disclosure, the 2.1.2 antigen binding domain comprises a heavy chain variable region and a light chain variable region. The heavy chain variable region of the 2.1.2 antigen binding domain may comprise, consist of, or consist essentially of the amino acid sequence of SEQ ID NO: 13. The light chain variable region of the 2.1.2 antigen binding domain may comprise, consist of, or consist essentially of the amino acid sequence of SEQ ID NO: 14. Accordingly, in aspects of the disclosure, the 2.1.2 antigen binding domain comprises a heavy chain variable region comprising the amino acid sequence of SEQ ID NO: 13 and/or a light chain variable region comprising the amino acid sequence of SEQ ID NO: 14. Preferably, the 2.1.2 antigen binding domain comprises the amino acid sequences of both SEQ ID NOs: 13 and 14.

In aspects of the disclosure, the GA101 antigen binding domain comprises a heavy chain variable region and a light chain variable region. The heavy chain variable region of the GA101 antigen binding domain may comprise, consist of, or consist essentially of the amino acid sequence of SEQ ID NO: 15. The light chain variable region of the GA101 antigen binding domain may comprise, consist of, or consist essentially of the amino acid sequence of SEQ ID NO: 16. Accordingly, in aspects of the disclosure, the GA101 antigen binding domain comprises a heavy chain variable region comprising the amino acid sequence of SEQ ID NO: 15 and/or a light chain variable region comprising the amino acid sequence of SEQ ID NO: 16. Preferably, the GA101 antigen binding domain comprises the amino acid sequences of both SEQ ID NOs: 15 and 16.

The inventive second CAR may comprise a 11B8 antigen binding domain comprising one or more of a light chain CDR1 comprising, consisting of, or consisting essentially of the amino acid sequence of SEQ ID NO: 17; a light chain CDR2 comprising, consisting of, or consisting essentially of the amino acid sequence of SEQ ID NO: 18; and a light chain CDR3 comprising, consisting of, or consisting essentially of the amino acid sequence of SEQ ID NO: 19. Preferably, the 11B8 light chain comprises all of the amino acid sequences of SEQ ID NOs: 17-19.

The inventive second CAR may comprise a 11B8 antigen binding domain comprising one or more of a heavy chain CDR1 comprising, consisting of, or consisting essentially of the amino acid sequence of SEQ ID NO: 20; a heavy chain CDR2 comprising, consisting of, or consisting essentially of the amino acid sequence of SEQ ID NO: 21; and a heavy chain CDR3 comprising, consisting of, or consisting essentially of the amino acid sequence of SEQ ID NO: 22. Preferably, the 11B8 heavy chain comprises all of the amino acid sequences of SEQ ID NOs: 20-22.

In aspects, the 11B8 antigen binding domain comprises the amino acid sequences of all of SEQ ID NOs: 17-22.

The inventive second CAR may comprise a GA101 antigen binding domain comprising one or more of a light chain CDR1 comprising, consisting of, or consisting essentially of the amino acid sequence of SEQ ID NO: 23; a light chain CDR2 comprising, consisting of, or consisting essentially of the amino acid sequence of SEQ ID NO: 24; and a light chain CDR3 comprising, consisting of, or consisting essentially of the amino acid sequence of SEQ ID NO: 25. Preferably, the GA101 light chain comprises all of the amino acid sequences of SEQ ID NOs: 23-25.

The inventive second CAR may comprise a GA101 antigen binding domain comprising one or more of a heavy chain CDR1 comprising, consisting of, or consisting essentially of the amino acid sequence of SEQ ID NO: 26; a heavy chain CDR2 comprising, consisting of, or consisting essentially of the amino acid sequence of SEQ ID NO: 27; and a heavy chain CDR3 comprising, consisting of, or consisting essentially of the amino acid sequence of SEQ ID NO: 28. Preferably, the GA101 heavy chain comprises all of the amino acid sequences of SEQ ID NOs: 26-28.

In aspects, the GA101 antigen binding domain comprises all of the amino acid sequences of SEQ ID NOs: 23-28.

CDR sequences can be determined by one of skill in the art as a routine matter. Such methods and available resources are known in the art, for example see Wu, et al., J. Exp. Med., 132: 211-250 (1970), IMG™, the international ImMunoGeneTics information system, and the freely available Paratome web server.

In aspects of the disclosure, the light chain variable region and the heavy chain variable region may be joined by an antigen binding domain linker peptide. The antigen binding domain linker peptide may be of any length and many comprise any amino acid sequence. For example, the antigen binding domain linker peptide may comprise or consist of any one or more of glycine, serine, lysine, proline, glutamic acid, and threonine, with or without other amino acid residues. In aspects of the disclosure, the antigen binding domain linker peptide may have a length of about 5 to about 100 amino acid residues, about 8 to about 75 amino acid residues, about 8 to about 50 amino acid residues, about 10 to about 25 amino acid residues, about 8 to about 30 amino acid residues, about 8 to about 40 amino acid residues, about 8 to about 50 amino acid residues, or about 12 to about 20 amino acid residues. In aspects of the disclosure, the antigen binding domain linker peptide has any of the foregoing lengths and consists of amino acid residues selected, independently, from the group consisting of glycine and serine. In aspects, the antigen binding domain linker peptide may comprise or consist of repeats of four glycines and one serine (G4S), for example, (G4S)³ (SEQ ID NO: 10). Such a linker could also have 4 repeats of (G4S), (G4S)⁴ (SEQ ID NO: 41). In aspects of the disclosure, the antigen binding domain linker peptide may comprise, consist, or consist essentially of, SEQ ID NO: 29 (GSTSGSGKPGSGEGSTKG). While the antigen binding domain may have a sequence from N-terminus to C-terminus of heavy-chain variable domain, linker, light-chain variable domain, in a preferred aspect, the antigen binding domain has a sequence from N-terminus to C-terminus of light-chain variable domain, linker, heavy-chain variable domain.

In another aspect, each of the first and second CARs comprises a leader sequence (also referred to as a signal sequence). The leader sequence may be positioned at the amino terminus of one or both of the first and second antigen binding domains (e.g., one or both of the light chain variable region of the anti-CD19 antibody and the anti-CD20 antibody). The leader sequence may be a human leader sequence. The leader sequence may comprise any suitable amino acid sequence. In one aspect, the leader sequence is a human granulocyte-macrophage colony-stimulating factor (GM-CSF) receptor leader sequence or a human CD8α leader sequence. For example, the antigen binding domain may comprise a human CD8α leader sequence comprising, consisting of, or consisting essentially of SEQ ID NO: 30. In aspects of the disclosure, while the leader sequence may facilitate expression of one or both of the first and second CARs on the surface of the cell, the presence of the leader sequence in one or both of the first and second expressed CARs may not be necessary in order for the CAR to function. In aspects of the disclosure, upon expression of one or both of the first and second CARs on the cell surface, all or a portion of the leader sequence may be cleaved off of the one or both of the first and second CARs. Accordingly, in aspects of the disclosure, the one or both of the first and second CARs lack a leader sequence.

In aspects of the disclosure, one or both of the first and second CARs comprise a hinge domain. One of ordinary skill in the art will appreciate that a hinge domain is a short sequence of amino acids that facilitates antibody flexibility (see, e.g., Woof et al., Nat. Rev. Immunol., 4(2): 89-99 (2004)). The hinge domain may be positioned between the antigen binding domain and the TM domain of one or both one or both of the first and second CARs. Preferably, the hinge domain is a human hinge domain. The hinge domain may comprise the hinge domain of human CD8α or human CD28. For example, the human hinge domain may comprise a sequence comprising, consisting of, or consisting essentially of the hinge domain of human CD8α.

The CAR may comprise a transmembrane (TM) domain. The TM domain can be any TM domain derived or obtained from any molecule known in the art. Preferably, the TM domain is a human TM domain. For example, the TM domain may comprise the TM domain of a human CD8α molecule or a human CD28 molecule. CD8 is a TM glycoprotein that serves as a co-receptor for the TCR, and is expressed primarily on the surface of cytotoxic T-cells. The most common form of CD8 exists as a dimer composed of a CD8α and CD8β chain. CD28 is expressed on T-cells and provides co-stimulatory signals for T-cell activation. CD28 is the receptor for CD80 (B7.1) and CD86 (B7.2). For example, the human TM domain may comprise a sequence comprising, consisting of, or consisting essentially of the TM domain of human CD8a.

The human CD8α hinge domain and human CD8α transmembrane domain may comprise, for example, a sequence comprising, consisting of, or consisting essentially of SEQ ID NO: 31. The nucleic acid may comprise, for example, a sequence comprising, consisting of, or consisting essentially of SEQ ID NO: 57 or 65.

One or both of the first and second CARs may comprise an intracellular (i.e., cytoplasmic) T-cell signaling domain. The intracellular T-cell signaling domain can be obtained or derived from a CD28 molecule, a CD3 zeta (ζ) molecule, an Fc receptor gamma (FcRγ) chain, a CD27 molecule, an OX40 molecule, a 4-1BB molecule, an inducible T-cell costimulatory protein (ICOS), or other intracellular signaling molecules known in the art, or modified versions of any of the foregoing. As discussed above, CD28 is a T-cell marker which is involved in T-cell co-stimulation. The intracellular T cell signaling domain of human CD28 may comprise, consist, or consist essentially of the amino acid sequence of SEQ ID NO: 32. The nucleic acid may comprise, consist, or consist essentially of the amino acid sequence of SEQ ID NO: 58 or 69. CD3ζ associates with TCRs to produce a signal and contains immunoreceptor tyrosine-based activation motifs (ITAMs). The intracellular T cell signaling domain of human CD3ζ may comprise, consist, or consist essentially of the amino acid sequence of SEQ ID NO: 33. The nucleic acid may comprise, consist, or consist essentially of the amino acid sequence of SEQ ID NO: 59 or 67. 4-1BB, also known as CD137, transmits a potent costimulatory signal to T-cells, promoting differentiation and enhancing long-term survival of T lymphocytes. The intracellular T cell signaling domain of human 4-1BB may comprise, consist, or consist essentially of the amino acid sequence of SEQ ID NO: 34. The nucleic acid may comprise, consist, or consist essentially of the amino acid sequence of SEQ ID NO: 66. ICOS is a CD28-superfamily costimulatory molecule that is expressed on activated T cells. In a preferred aspect, the CD28, CD3ζ, FcRγ, ICOS, 4-1BB, OX40, and CD27 are human.

One or both of the first and second CARs can comprise any one or more of the aforementioned TM domains and any one or more of the aforementioned intracellular T-cell signaling domains in any combination. For example, the inventive first CAR may comprise a CD8α hinge and TM domain and intracellular T-cell signaling domains of CD28 and CD3ζ. Alternatively, for example, the inventive second CAR may comprise a CD8α hinge and TM domain and intracellular T-cell signaling domains of 4-1BB and CD3ζ.

In aspects, the inventive CAR construct encodes, from the amino terminus to the carboxyl terminus, a CD8α leader sequence, an anti-CD19 scFv, human CD8α hinge and transmembrane domains, an intracellular T cell signaling domain of human CD28, an intracellular T cell signaling domain of the human CD3ζ molecule, a cleavage sequence, a CD8α leader sequence, an anti-CD20 scFv, human CD8α hinge and transmembrane domains, 4-1BB intracellular T cell signaling domain, and an intracellular T cell signaling domain of the human CD3ζ molecule.

In aspects, the inventive first CAR comprises from the amino terminus to the carboxyl terminus, a leader sequence, an anti-CD19 scFv, human CD8α hinge and transmembrane domains, an intracellular T cell signaling domain of human CD28, and an intracellular T cell signaling domain of the human CD3ζ molecule.

In aspects, the inventive second CAR comprises from the amino terminus to the carboxyl terminus, a leader sequence, an anti-CD20 scFv, a human CD8α hinge and transmembrane domains, 4-1BB intracellular T cell signaling domain, and an intracellular T cell signaling domain of the human CD3ζ molecule.

Included in the scope of the disclosure are functional portions of the inventive CARs described herein. The term “functional portion” when used in reference to a CAR refers to any part or fragment of the CAR of the disclosure, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, about 25%, about 30%, about 50%, about 60%, about 70%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the inventive CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 98% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The CARs of aspects of the disclosure (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 1000 amino acids long, such as 50, 70, 75, 100, 125, 150, 175, 200, 300, 400, 500, 600, 700, 800, 900, 1000 or more amino acids in length.

The CARs of aspects of the disclosure (including functional portions and functional variants of the disclosure) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N‘-benzyl-N’-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α, γ-diaminobutyric acid, α,β-diaminopropionic acid, homophenylalanine, and α-tert-butylglycine.

The CARs of aspects of the disclosure (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs of aspects of the disclosure (including functional portions and functional variants thereof) can be obtained by methods known in the art. The CARs may be made by any suitable method of making polypeptides or proteins. For example, CARs can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the CARs described herein (including functional portions and functional variants thereof) can be commercially synthesized by commercial entities. In this respect, the inventive CARs can be synthetic, recombinant, isolated, and/or purified.

Further provided by an aspect of the disclosure is a nucleic acid comprising a nucleotide sequence encoding any of the CARs described herein (including functional portions and functional variants thereof). The nucleic acids of the disclosure may comprise a nucleotide sequence encoding any of the leader domains, hinge domains, antigen binding domains, cleavage sequences, TM domains, and intracellular T cell signaling domains described herein.

In aspects of the disclosure, the first and/or second CAR may be provided in combination with a regulatory element capable of modulating the activity of a host cell expressing the CAR. For example, the regulatory element may regulate the anti-CD19 and/or anti-CD20 activity of a host cell expressing the CAR. The regulatory element may regulate the anti-CD19 and/or anti-CD20 activity of a host cell expressing the first and/or second CAR. For example, the regulatory element may act as an “on” or “off” switch.

In aspects of the disclosure, the regulatory element downregulates the activity, e.g., the anti-CD19 and/or anti-CD20 activity, of the host cell expressing the first and/or second CAR. For example, the regulatory element kills the host cell expressing the first and/or second CAR. In this regard, the regulatory element is a suicide gene. In aspects of the disclosure, the regulatory element is an inducible dimerization kill switch. An example of an inducible dimerization kill switch is the IC9 suicide gene. Another example of an inducible dimerization kill switch is an element which provides for small-molecule-induced dimerization of the intracellular signaling domain of Fas, which induces apoptosis via a caspase-8-dependent pathway. This approach may be used to induce apoptosis using a small molecule made by fusing two molecules of the drug calcineurin (Spencer et al., Curr. Biol., 6: 839-47 (1996); Belshaw et al., Chem. Biol., 3: 731-38 (1996)) or the FKBP/AP1903 dimerizer system described herein (Thomis et al., Blood, 97: 1249-57 (2001)).

In aspects of the disclosure, the regulatory element is a cell surface marker. The cell surface marker may be co-expressed with the first and/or second CAR. Administration of an antibody targeting the cell surface marker may reduce or eliminate the first and/or second CAR-expressing host cells. Such cell surface markers may be useful as a safety mechanism to deplete CAR-positive cells in vivo. In vivo depletion may occur by one or both of complement-mediated lysis of opsonized cells and antibody-mediated cell-dependent cytotoxicity. For example, cells transduced with a cell surface marker which is a CD8α stalk with two rituximab (anti-CD20) mimotopes can be depleted with rituximab (Philip et al., Blood, 124: 1277-87 (2014)). Other examples of cell surface markers which may be targeted for depletion by an antibody include CD20 (Griffioen et al., Haematologica, 94: 1316-20 (2009)), c-myc epitope tag (Kieback et al., PNAS, 105: 623-28 (2008)), and truncated versions of the human epidermal growth factor receptor. The truncated epidermal growth factor receptor may lack one or both of the ligand-binding and intracellular signaling domains but retain the epitope for cetuximab binding (Wang et al., Blood, 118: 1255-63 (2011)).

The regulatory element may be an inhibitory receptor. For example, antigen-specific inhibitory chimeric antigen receptors (iCARs) may preemptively constrain T cell responses. Such iCARs may selectively limit cytokine secretion, cytotoxicity, and proliferation induced through the endogenous T cell receptor or an activating chimeric receptor (Fedorov et al., Sci. Transl. Med., 5:215ra172 (2013)).

In aspects of the disclosure, the regulatory element upregulates the activity, e.g., anti-CD19 and/or anti-CD20 activity of the host cell. In this regard, the regulatory element may act as an “on” switch to control expression or activity of the first and/or second CAR to occur where and when it is needed.

For example, the regulatory element may be an element which confers dependence on small-molecule ligands for cell survival or activity. An example of such an element may be a drug-responsive, ribozyme-based regulatory device linked to growth cytokine targets to control cell (e.g., T cell) proliferation (Chen et al., PNAS, 107(19): 8531-6 (2010)). Another example may be to design the antigen-binding and intracellular signaling components of the CAR to assemble only in the presence of a heterodimerizing small molecule (Wu et al., Science, 350(6258):aab4077 (2015)).

Other potential regulatory elements may include elements which control the location of transgene integration (Schumann et al., PNAS, 112(33): 10437-42 (2015)) or a genetic deletion which produces an auxotrophic cell (e.g., T cell).

In another aspect of the disclosure, the nucleotide sequence encoding the first and/or second CAR is RNA. Introducing CAR mRNA into cells may result in transient expression of the CAR. With this approach, the mRNA may persist for a few days, but there may be an antitumor effect with minimal on-target toxicity (Beatty et al., Cancer Immunol. Res., 2(2): 112-20 (2014)).

In aspects of the disclosure, the first and/or second CAR is provided in combination with a suicide gene. The product of the suicide gene may, advantageously, provide on-demand reduction or elimination of host cells.

As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, inducible caspase 9 (IC9) gene, purine nucleoside phosphorylase, and nitroreductase.

The suicide gene may be the IC9 gene. The product of the IC9 gene contains part of the proapoptotic protein human caspase 9 (“caspase 9 component”) fused to a binding domain derived from human FK-506 binding protein (FKBP12 component). Activation of the caspase 9 domain of IC9 is dependent on dimerization of IC9 proteins that occurs when a small molecule drug, rimiducid (AP1903), binds to the FKBP12 moiety of IC9. After caspase 9 is activated, the cells carrying the IC9 gene undergo apoptosis.

In aspects of the disclosure, the nucleic acid comprises a nucleotide sequence encoding a cleavage sequence that is positioned between the first and second CARs. In aspects of the disclosure, the cleavage sequence is cleavable. In this regard, the amino acid sequence encoded by the inventive nucleic acids may be cleaved such that two proteins are produced: a first protein encoded by the nucleotide sequence encoding the first CAR and a second protein encoded by the nucleotide sequence encoding the second CAR.

In aspects, the cleavable cleavage sequence comprises a “self cleaving” sequence. In aspects, the “self cleaving” sequence is a “self cleaving” 2A peptide. “Self cleaving” 2A peptides are described, for example, in Liu et al., Sci. Rep., 7(1): 2193 (2017), and Szymczak et al., Nature Biotechnol., 22(5): 589-594 (2004). 2A peptides are viral oligopeptides that mediate cleavage of polypeptides during translation in eukaryotic cells. The designation “2A” refers to a specific region of the viral genome. Without being bound to a particular theory or mechanism, it is believed that the mechanism of 2A-mediated “self cleavage” is ribosome skipping of the formation of a glycyl-prolyl peptide bond at the C-terminus of the 2A peptide. Different 2A peptides may comprise, at the C-terminus, the consensus amino acid sequence of GDVEX₁NPGP (SEQ ID NO: 35), wherein X₁ of SEQ ID NO: 35 is any naturally occurring amino acid residue. In aspects of the disclosure, the cleavable ribosomal skip sequence is a porcine teschovirus-1 2A (P2A) amino acid sequence, equine rhinitis A virus (E2A) amino acid sequence, thosea asigna virus 2A (T2A) amino acid sequence, or foot-and-mouth disease virus (F2A) amino acid sequence. In aspects of the disclosure, the ribosomal skip sequence is a 2A peptide amino acid sequence comprising, consisting, or consisting essentially of, the amino acid sequence of (F2A).

In aspects, the cleavable cleavage sequence comprises an enzyme-cleavable sequence. In aspects, the enzyme-cleavable sequence is a furin-cleavable sequence. Exemplary furin-cleavable sequences are described in Duckert et al., Protein Engineering, Design & Selection, 17(1): 107-112 (2004) and U.S. Pat. No. 8,871,906, each of which is incorporated herein by reference. In aspects of the disclosure, the furin-cleavable sequence is represented by the formula P4-P3-P2-P1 (Formula I), wherein P4 is an amino acid residue at the amino end, P1 is an amino acid residue at the carboxyl end, P1 is an arginine or a lysine residue, and the sequence is cleavable at the carboxyl end of P1 by furin. In another aspect of the disclosure, the furin-cleavable sequence of Formula I (i) further comprises amino acid residues represented by P6-P5 at the amino end, (ii) further comprises amino acid residues represented by P1′-P2′ at the carboxyl end, (iii) wherein if P1 is an arginine or a lysine residue, P2′ is tryptophan, and P4 is arginine, valine or lysine, provided that if P4 is not arginine, then P6 and P2 are basic residues, and (iv) the sequence is cleavable at the carboxyl end of P1 by furin. In aspects of the disclosure, the furin-cleavable sequence comprises R-X₁-X₂-R, wherein X₁ is any naturally occurring amino acid and X₂ is arginine or lysine.

In aspects of the disclosure, the cleavage sequence comprises an enzyme-cleavable sequence and any “self cleaving” sequence. In aspects of the disclosure, the cleavage sequence comprises an enzyme-cleavable sequence (e.g., a furin cleavable sequence), a spacer (e.g., SGSG [SEQ ID NO: 36]), and a “self cleaving” sequence (e.g., F2A). In aspects of the disclosure, the cleavage sequence is an amino acid sequence comprising, consisting, or consisting essentially of, the amino acid sequence of (SEQ ID NO: 37).

Another aspect of the disclosure provides a nucleic acid comprising a nucleotide sequence encoding an anti-CD19 CAR comprising an antigen binding domain, a TM domain, and an intracellular T cell signaling domain, wherein the antigen binding domain has antigenic specificity for CD19. The anti-CD19 CAR may be as described herein with respect to other aspects of the disclosure.

In aspects, the disclosure provides a nucleic acid comprising a nucleotide sequence encoding a single CAR of a CAR construct wherein the nucleic acid of the CAR construct has been designed to reduce retroviral recombination. Such a single CAR may be as described herein with respect to other aspects of the disclosure.

A further aspect of the disclosure provides a nucleic acid, wherein the CAR construct comprises exactly two CARs being the first and second CARs, respectively.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some aspects, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

The nucleic acids of an aspect of the disclosure may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green and Sambrook, supra. The nucleic acids can be constructed based on chemical synthesis and/or enzymatic ligation reactions using procedures known in the art. See, for example, Green and Sambrook, supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the disclosure can be purchased from commercial entities.

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An aspect of the disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs (alone or in combination with a suicide gene). It is generally appreciated that conditions can be rendered more stringent by the addition of increasing amounts of formamide.

The disclosure also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In aspects, the nucleic acids of the disclosure can be incorporated into a recombinant expression vector. In this regard, an aspect of the disclosure provides recombinant expression vectors comprising any of the nucleic acids of the disclosure. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the disclosure are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In aspects, the recombinant expression vector of the disclosure can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBIl21 and pBIN19 (Clontech). Examples of animal expression vectors include pEUK-Cl, nMAM, and pMAMneo (Clontech). The recombinant expression vector may be a viral vector, e.g., a retroviral vector (e.g., a gamma-retroviral vector) or a lentiviral vector.

In aspects, the recombinant expression vectors of the disclosure can be prepared using standard recombinant DNA techniques described in, for example, Green and Sambrook, supra. Constructs of expression vectors, which are circular or linear, can be prepared to contain a replication system functional in a prokaryotic or eukaryotic host cell. Replication systems can be derived, e.g., from ColEl, 2 plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning. In addition to the inventive nucleic acid sequence encoding the CARs (alone or in combination with a suicide gene), the recombinant expression vector preferably comprises expression control sequences, such as promoters, enhancers, polyadenylation signals, transcription terminators, internal ribosome entry sites (IRES), and the like, that provide for the expression of the nucleic acid sequence in a host cell.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CARs (including functional portions and functional variants thereof) (alone or in combination with a suicide gene), or to the nucleotide sequence which is complementary to or which hybridizes to the nucleotide sequence encoding the CARs (alone or in combination with a suicide gene). The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

An aspect of the disclosure further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, the host cell may be a mammalian cell. The host cell may be a human cell. The host cell can be of any cell type, can originate from any type of tissue, and can be of any developmental stage. The host cell may be a peripheral blood lymphocyte (PBL) or a peripheral blood mononuclear cell (PBMC) or a macrophage.

In aspects of the disclosure, the host cell is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell.

In aspects of the disclosure, the host cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 9^{th} ed., Janeway et al., eds., Garland Publishing, New York, NY (2016)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be involved in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

Also provided by an aspect of the disclosure is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the disclosure, the population of cells is a clonal population comprising host cells comprising a recombinant expression vector as described herein.

The inventive recombinant expression vectors encoding the CARs may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Phage or viral vectors can be introduced into host cells, after growth of infectious particles in suitable packaging cells, many of which are commercially available.

Included in the scope of the disclosure are conjugates, e.g., bioconjugates, comprising any of the inventive CARs (including any of the functional portions or variants thereof), nucleic acids, recombinant expression vectors, host cells, or populations of host cells. Conjugates, as well as methods of synthesizing conjugates in general, are known in the art.

CARs (including functional portions and variants thereof) (alone or in combination with a suicide gene product), nucleic acids, systems, protein(s) and combination(s) of proteins encoded by the nucleic acids, recombinant expression vectors, and host cells (including populations thereof), all of which are collectively referred to as “inventive CAR materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some aspects, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The inventive CAR materials can be formulated into a composition, such as a pharmaceutical composition. In this regard, an aspect of the disclosure provides a pharmaceutical composition comprising any of the inventive CAR materials and a pharmaceutically acceptable carrier. The inventive pharmaceutical compositions containing any of the inventive CAR materials can comprise more than one inventive CAR material, e.g., a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, cyclophosphamide, daunorubicin, doxorubicin, fludarabine, fluorouracil, gemcitabine, hydroxyurea, methotrexate, paclitaxel, rituximab, vinblastine, vincristine, etc. In a preferred aspect, the pharmaceutical composition comprises the inventive host cell or populations thereof.

Preferably, the carrier is a pharmaceutically acceptable carrier. With respect to pharmaceutical compositions, the carrier can be any of those conventionally used for the particular inventive CAR material under consideration. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular inventive CAR material, as well as by the particular method used to administer the inventive CAR material. In a preferred aspect, the CARs are expressed by a host cell, which is preferably a T cell or an NK cell, and host cells expressing the CARs are administered to a patient. These cells could be autologous or allogeneic in relation to the recipient of the cells. A nucleic acid encoding the CARs may be introduced to the cells by any of a variety of methods of genetic modification including, but not limited to, transduction with a gamma-retrovirus, a lentivirus, or a transposon system. There are a variety of suitable formulations of the pharmaceutical composition of the disclosure. Suitable formulations may include any of those for parenteral, subcutaneous, intravenous, intramuscular, intratumoral, intraarterial, intrathecal, or interperitoneal administration. More than one route can be used to administer the inventive CAR materials, and in certain instances, a particular route can provide a more immediate and more effective response than another route.

Preferably, the inventive CAR material is administered by injection, e.g., intravenously. When the inventive CAR material is a host cell expressing the inventive CARs (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In aspects, the pharmaceutically acceptable carrier is supplemented with human serum albumen.

The composition can employ time-released, delayed release, and sustained release delivery systems such that the delivery of the inventive composition occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition aspects of the disclosure.

Without being bound to a particular theory or mechanism, it is believed that by eliciting an antigen-specific response against CD19 and/or CD20, the first and/or second CARs provide for one or more of the following: targeting and destroying CD19 and/or CD20-expressing cancer cells, reducing or eliminating cancer cells, facilitating infiltration of immune cells to tumor site(s), and enhancing/extending anti-cancer responses.

It is contemplated that the first and/or second CARs materials can be used in methods of treating or preventing a disease, e.g., cancer, in a mammal. Without being bound to a particular theory or mechanism, the first and/or second CARs have biological activity, e.g., ability to recognize antigen, e.g., CD19 and/or CD20, such that the first and/or second CAR when expressed by a cell is able to mediate an immune response against the cell expressing the antigen, e.g., CD19 and/or CD20, for which the first and/or second CAR is specific. In this regard, an aspect of the disclosure provides a method of treating or preventing cancer in a mammal, comprising administering to the mammal any of the CARs (including functional portions and variants thereof) (alone or in combination with a suicide gene product), nucleic acids, systems, protein(s) (including combination(s) of proteins) encoded by the nucleic acids, recombinant expression vectors, host cells (including populations thereof) and/or pharmaceutical compositions of the disclosure in an amount effective to treat or prevent cancer in the mammal. In a preferred aspect, the method comprises infusing the mammal with host cells transduced with the inventive CAR construct.

One or more isolated host cells expressing the first and/or second CARs described herein can be contacted with a population of cancer cells that express CD19 and/or CD20 ex vivo, in vivo, or in vitro. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. The inventive method preferably involves ex vivo and in vivo components. In this regard, for example, the isolated host cells described above can be cultured ex vivo under conditions to express the first and/or second CARs, and then directly transferred into a mammal (preferably a human) affected by a CD19 and/or CD20-positive cancer, e.g., lymphoma. Such a cell transfer method is referred to in the art as “adoptive cell transfer (ACT),” in which immune-derived cells are transferred into a recipient to transfer the functionality of the immune-derived cells to the host. The immune-derived cells may have originated from the recipient or from another individual. Adoptive cell transfer methods may be used to treat various types of cancers, including hematological cancers such as myeloma.

Once the composition comprising host cells expressing the inventive first and second CAR-encoding nucleic acid sequence, or a vector comprising the inventive first and second CAR-encoding nucleic acid sequence, is administered to a mammal (e.g., a human), the biological activity of the first and/or second CAR can be measured by any suitable method known in the art. In accordance with the inventive method, the first CAR binds to, e.g., CD19 and/or the second CAR binds to, e.g., CD20 on the cancer, and the cancer cells are destroyed. Binding of the first CAR to CD19 and/or the second CAR to CD20 on the surface of cancer cells can be assayed using any suitable method known in the art, including, for example, ELISA (enzyme-linked immunosorbent assays) and flow cytometry. The ability of the CARs to destroy cells can be measured using any suitable method known in the art, such as cytotoxicity assays described in, for example, Kochenderfer et al., J. Immunotherapy, 32(7): 689-702 (2009), and Herman et al. J. ImmunologicalMethods, 285(1): 25-40 (2004). The biological activity of the first and/or second CAR also can be measured by assaying expression of certain cytokines, such as CD107α, IFNγ, 1L-2, and TNF.

An aspect of the disclosure further comprises lymphodepleting the mammal prior to administering the inventive CAR material. Examples of lymphodepletion include, but may not be limited to, nonmyeloablative lymphodepleting chemotherapy, myeloablative lymphodepleting chemotherapy, total body irradiation, etc. For example, a lymphodepleting chemotherapy regimen can be administered to the mammal prior to administering the inventive CAR material to the mammal. In aspects, cyclophosphamide and/or fludarabine are administered to a mammal prior to administering the inventive CAR material. In aspects, cyclophosphamide and/or fludarabine are administered for three consecutive days to a mammal prior to administering the inventive CAR material. In a further aspect, cyclophosphamide is administered at a dose of from about 1 to about 100 mg/m² (e.g., from about 50 to about 950, from about 100 to about 900, from about 200 to about 800, from about 300 to about 700, from about 400 to about 600, from about 450 to about 550, from about 300 to about 500, about 300, about 400, or about 500 mg/m²). In a further aspect, fludarabine is administered at a dose of from about 1 to about 100 mg/m² (e.g., from about 5 to about 80, from about 10 to about 70, from about 15 to about 60, from about 20 to about 50, from about 25 to about 40, from about 27 to about 33, or about 30 mg/m²). In some aspects, the inventive CAR material can be administered (e.g., infused) about 72 hours after the last dose of chemotherapy.

For purposes of the inventive methods, wherein host cells or populations of cells are administered, the cells can be cells that are allogeneic or autologous to the mammal. Preferably, the cells are autologous to the mammal.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the particular CAR material selected, method of administration, timing and frequency of administration, the existence, nature, and extent of any adverse side-effects that might accompany the administration of a particular CAR material, and the desired physiological effect. It will be appreciated by one of skill in the art that various diseases or disorders (e.g., cancer) could require prolonged treatment involving multiple administrations, perhaps using the inventive CAR materials in each or various rounds of administration. By way of example and not intending to limit the disclosure, the dose of the inventive CAR material can be about 0.001 to about 1000 mg/kg body weight of the subject being treated/day, from about 0.01 to about 10 mg/kg body weight/day, about 0.01 mg to about 1 mg/kg body weight/day. In aspects of the disclosure, the dose may be from about 1×10⁴ to about 1×10¹⁰ cells expressing the first and/or second CAR per kg body weight. When the inventive CAR material is a host cell, an exemplary dose of host cells may be a minimum of one million cells (1 million cells/dose to as many as 10¹¹ cells/dose), e.g., 1×10′ cells. When the inventive CAR material is a nucleic acid packaged in a virus, an exemplary dose of virus may be 1 ng/dose.

For purposes of the disclosure, the amount or dose of the inventive CAR material administered should be sufficient to effect a therapeutic or prophylactic response in the subject or animal over a reasonable time frame. For example, the dose of the inventive CAR material should be sufficient to bind to antigen, or detect, treat or prevent disease, e.g., cancer, in a period of from about 2 hours or longer, e.g., about 12 to about 24 or more hours, from the time of administration. In certain aspects, the time period could be even longer. The dose will be determined by the efficacy of the particular inventive CAR material and the condition of the animal (e.g., human), as well as the body weight of the animal (e.g., human) to be treated.

For purposes of the disclosure, an assay, which comprises, for example, comparing the extent to which target cells are lysed and/or IFNγ is secreted by T cells expressing the first and/or second CAR upon administration of a given dose of such T cells to a mammal, among a set of mammals of which is each given a different dose of the T cells, could be used to determine a starting dose to be administered to a mammal. The extent to which target cells are lysed and/or IFNγ is secreted upon administration of a certain dose can be assayed by methods known in the art.

When the inventive CAR materials are administered with one or more additional therapeutic agents, one or more additional therapeutic agents can be coadministered to the mammal. By “coadministering” is meant administering one or more additional therapeutic agents and the inventive CAR materials sufficiently close in time such that the inventive CAR materials can enhance the effect of one or more additional therapeutic agents, or vice versa. In this regard, the inventive CAR materials can be administered first and the one or more additional therapeutic agents can be administered second, or vice versa. Alternatively, the inventive CAR materials and the one or more additional therapeutic agents can be administered simultaneously. An exemplary therapeutic agent that can be co-administered with the CAR materials is IL-2. It is believed that IL-2 enhances the therapeutic effect of the inventive CAR materials. Without being bound by a particular theory or mechanism, it is believed that IL-2 enhances therapy by enhancing the in vivo expansion of the numbers of cells expressing the first and/or second CARs.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods, the cancer can be any cancer. In aspects of the disclosure, the cancer is a CD19 and/or CD20-expressing cancer. In aspects of the disclosure, the cancer is leukemia and/or lymphoma.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, e.g., cancer, or a symptom or condition thereof or preventing the recurrence of the disease, e.g., cancer.

Another aspect of the disclosure provides any of the first and/or second CARs (including functional portions and variants thereof) (alone or in combination with a suicide gene product), nucleic acids, systems, protein(s) (including combination(s) of proteins) encoded by the nucleic acids, recombinant expression vectors, host cells (including populations thereof) and/or pharmaceutical compositions described herein with respect to other aspects of the disclosure for use in a method of treating or preventing cancer in a mammal. Still another aspect of the disclosure provides the use of any of the first and/or second CARs (including functional portions and variants thereof) (alone or in combination with a suicide gene product), nucleic acids, systems, protein(s) (including combination(s) of proteins) encoded by the nucleic acids, recombinant expression vectors, host cells (including populations thereof) and/or pharmaceutical compositions described herein with respect to other aspects of the disclosure in the manufacture of a medicament for the treatment or prevention of cancer in a mammal. The cancer may be any of the cancers described herein.

A further aspect of the disclosure provides one or more polypeptide(s) encoded by the nucleic acids of the disclosure.

Another aspect of the disclosure provides methods of detecting the presence of cancer in a mammal, comprising (a) contacting a sample comprising one or more cells from the mammal with nucleic acids, protein(s) (including combination(s) of proteins) encoded by the nucleic acids, recombinant expression vectors, host cells (including populations thereof) and/or pharmaceutical compositions of the disclosure, thereby forming a complex, and (b) detecting the complex, wherein detection of the complex is indicative of the presence of cancer in the mammal.

In aspects, the disclosure provides a method of making a chimeric antigen receptor (CAR) construct, the method comprising: (i) designing a nucleic acid comprising a nucleotide sequence encoding the CAR construct comprising (a) a first CAR comprising a first antigen binding domain, a first transmembrane domain, and a first intracellular T cell signaling domain; (b) a second CAR comprising a second antigen binding domain, a second transmembrane domain, and a second intracellular T cell signaling domain; and (c) a cleavage sequence; wherein the cleavage sequence is positioned between the first and second CARs; (ii) designing the nucleic acid to reduce retroviral recombination; and (iii) preparing the nucleic acid of (ii). Preparation of the nucleic acid may be of any suitable means known in the art, e.g., such as methods described above, including, for example, as described in Green and Sambrook, supra.

In aspects, the nucleic acid sequence identity between the first and second CARs is no more than 99%, 98%, 97%, 96%, 95%, 94%, 93%, 92%, 91%, 90%, 89%, 88%, 87%, 86%, 85%, 84%, 83%, 82%, 81%, or 80%. In aspects, the nucleic acid sequence identity between the first and second CARs is no more than 90%. In aspects, the nucleic acid, when expressed in a host cell, exhibits greater expression compared to a nucleic acid that encodes the same amino acid sequence but that has not been designed to reduce retroviral recombination. In aspects, the method further comprises expressing the CAR construct in a host cell.

The following includes certain aspects of the disclosure.

1. A nucleic acid comprising a nucleotide sequence encoding a chimeric antigen receptor (CAR) construct comprising:
(a) a first CAR comprising
   a first antigen binding domain,
   a first transmembrane domain, and
   a first intracellular T cell signaling domain;
(b) a second CAR comprising
   a second antigen binding domain,
   a second transmembrane domain, and
   a second intracellular T cell signaling domain; and
(c) a cleavage sequence,
   wherein the cleavage sequence is positioned between the first and second CARs; and
   wherein the nucleic acid has been designed to reduce retroviral recombination.

2. The nucleic acid of aspect 1, wherein the nucleic acid sequence identity between the first and second CARs is no more than 90%.

3. The nucleic acid of aspect 1 or 2, wherein the nucleic acid, when expressed in a host cell, exhibits greater expression compared to a nucleic acid that encodes the same amino acid sequence but that has not been designed to reduce retroviral recombination.

4. A nucleic acid comprising a nucleotide sequence encoding a chimeric antigen receptor (CAR) construct comprising:
(a) a first CAR comprising
   a first antigen binding domain,
   a first transmembrane domain, and
   a first intracellular T cell signaling domain;
(b) a second CAR comprising
   a second antigen binding domain,
   a second transmembrane domain, and
   a second intracellular T cell signaling domain; and
(c) a cleavage sequence,
   wherein the cleavage sequence is positioned between the first and second CARs; and
(d) wherein the first or second antigen binding domain comprises a linker of SEQ ID NO: 41.

5. The nucleic acid of any one of aspects 1-4, wherein the first antigen binding domain of the first CAR has antigenic specificity for CD19, and wherein the second antigen binding domain of the second CAR has antigenic specificity for CD20.

6. The nucleic acid of any one of aspects 1-5, wherein the cleavage sequence comprises any one of the following: porcine teschovirus-1 2A (P2A) amino acid sequence, equine rhinitis A virus (E2A) amino acid sequence, thosea asigna virus 2A (T2A) amino acid sequence, foot-and-mouth disease virus (F2A) amino acid sequence, or a furin-cleavable amino acid sequence, modified versions of any of the foregoing, or any combination of the foregoing.

7. The nucleic acid of any one of aspects 1-6, wherein the cleavage sequence comprises a foot-and-mouth disease virus (F2A) amino acid sequence.

8. The nucleic acid of any one of aspects 1-7, wherein the cleavage sequence comprises an amino acid sequence comprising SEQ ID NO: 37.

9. The nucleic acid of any one of aspects 1-8, wherein the first antigen binding domain comprises the six CDRs of Hul9 or 47G4.

10. The nucleic acid of any one of aspects 1-9, wherein the first antigen binding domain comprises single-chain variable fragment Hul9.

11. The nucleic acid of any one of aspects 1-10, wherein the second antigen binding domain comprises the six CDRs of 11B8, C2B8, 2.1.2, 8G6, or GA101.

12. The nucleic acid of any one of aspects 1-10, wherein the second antigen binding domain comprises an antigen binding domain of antibody C2B, 11B8, 8G6, 2.1.2, or GA101.

13. The nucleic acid of any one of aspects 1-12, wherein one or both of the first and second transmembrane domain(s) comprises a CD8 transmembrane domain and hinge domain.

14. The nucleic acid of aspect 13, wherein one or both of the first and second CARs comprises the nucleic acid sequence of SEQ ID NO: 57 or 65.

15. The nucleic acid of any one of aspects 1-14, wherein one or both of the first and second intracellular T cell signaling domain(s) comprises any one of the following: a human CD28 protein, a human CD3-zeta protein, a human FcRy protein, a CD27 protein, an OX40 protein, a human 4-1BB protein, a human inducible T-cell costimulatory protein (ICOS), modified versions of any of the foregoing, or any combination of the foregoing.

16. The nucleic acid of any one of aspects 1-15, wherein one or both of the first and second intracellular T cell signaling domain(s) comprises a CD28 intracellular T cell signaling sequence or a 41BB sequence.

17. The nucleic acid of aspect 16, wherein one or both of the first and second intracellular T cell signaling domain(s) comprises a CD28 intracellular T cell signaling sequence comprising the nucleic acid sequence of SEQ ID NO: 58 or 69; or wherein one or both of the first and second intracellular T cell signaling domain(s) comprises a 4-1BB intracellular T cell signaling sequence comprising the nucleic acid sequence of SEQ ID NO: 66.

18. The nucleic acid of any one of aspects 1-17, wherein one or both of the first and second intracellular T cell signaling domain(s) comprises a CD3 zeta (( ) intracellular T cell signaling sequence.

19. The nucleic acid of aspect 18, wherein the CD3 intracellular T cell signaling sequence comprises the nucleic acid sequence of SEQ ID NO: 59 or 67.

20. The nucleic acid of any one of aspects 1-19, wherein the CAR construct comprises a CD8 leader domain.

21. The nucleic acid of aspect 20, wherein the CD8 leader domain sequence comprises the nucleic acid sequence of SEQ ID NO: 53.

22. The nucleic acid of any one of aspects 1-21, wherein the CAR construct comprises exactly two CARs being the first and second CARs, respectively.

23. The nucleic acid of aspect 1, comprising the nucleic acid sequence of one or more of SEQ ID NOs: 42-45.

24. A nucleic acid comprising the nucleic acid sequence of one or more of SEQ ID NOs: 48-52.

25. A chimeric antigen receptor (CAR) comprising the amino acid sequence of any one of SEQ ID NOs: 71-79.

26. The CAR of aspect 25, wherein the CAR comprises the amino acid sequence of SEQ ID NO: 72.

27. A recombinant expression vector comprising the nucleic acid of any one of aspects 1-24.

28. The recombinant expression vector of aspect 27, wherein the vector is a gamma-retrovirus, lentivirus, or transposon vector.

29. An isolated host cell comprising the recombinant expression vector of aspect 27 or 28.

30. The isolated host cell of aspect 29, wherein the cell is a T cell, a macrophage, or a NK cell.

31. A population of cells comprising at least one host cell of aspect 29 or 30.

32. A pharmaceutical composition comprising the nucleic acid of any one of aspects 1-24, the CAR of aspect 25 or 26, the recombinant expression vector of aspect 27 or 28, the host cell of aspect 29 or 30, or the population of cells of aspect 31, and a pharmaceutically acceptable carrier.

33. A method of detecting the presence of cancer in a mammal, comprising:
(a) contacting a sample comprising one or more cells from the mammal with the nucleic acid of any one of aspects 1-24, the CAR of aspect 25 or 26, the recombinant expression vector of aspect 27 or 28, the host cell of aspect 29 or 30, or the population of cells of aspect 31, or the pharmaceutical composition of aspect 32, thereby forming a complex, and
(b) detecting the complex, wherein detection of the complex is indicative of the presence of cancer in the mammal.

34. The nucleic acid of any one of aspects 1-24, the CAR of aspect 25 or 26, the recombinant expression vector of aspect 27 or 28, the host cell of aspect 29 or 30, or the population of cells of aspect 31, or the pharmaceutical composition of aspect 32 for use in the treatment or prevention of cancer in a mammal.

35. The host cell of aspect 29 or 30 or the population of cells of aspect 31 for the use of aspect 34.

36. The host cell of aspect 29 or 30 or the population of cells of aspect 31 for the use of aspect 34 or 35, wherein the host cell or population of cells is autologous in relation to the mammal.

37. The host cell of aspect 29 or 30 or the population of cells of aspect 31 for the use of aspect 34 or 35, wherein the host cell or population of cells is allogeneic in relation to the mammal.

38. The nucleic acid of any one of aspects 1-24, the CAR of aspect 25 or 26, the recombinant expression vector of aspect 27 or 28, the host cell of aspect 29 or 30, or the population of cells of aspect 31, or the pharmaceutical composition of aspect 32, for the use of aspect 34 or 35, wherein the cancer is a hematological malignancy.

39. A method of making a chimeric antigen receptor (CAR) construct, the method comprising:
(i) designing a nucleic acid comprising a nucleotide sequence encoding a CAR construct comprising
   (a) a first CAR comprising
      a first antigen binding domain,
      a first transmembrane domain, and
      a first intracellular T cell signaling domain;
   (b) a second CAR comprising
      a second antigen binding domain,
      a second transmembrane domain, and
      a second intracellular T cell signaling domain; and
   (c) a cleavage sequence, wherein the cleavage sequence is positioned between the first and second CARs;
   (ii) designing the nucleic acid to reduce retroviral recombination; and
   (iii) preparing the nucleic acid of (ii).

40. The method of aspect 39, wherein the sequence identity between the first and second CARs is no more than 90%.

41. The method of aspect 39 or 40, wherein the nucleic acid, when expressed in a host cell, exhibits greater expression compared to a nucleic acid that encodes the same amino acid sequence but that has not been designed to reduce retroviral recombination.

42. The method of any one of aspects 39-41, wherein the method further comprises expressing the CAR construct in a host cell.

43. A method of making a CAR comprising a nucleic acid sequence of any one of SEQ ID NOs: 48-52.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

The following examples further illustrate the disclosure but, of course, should not be construed as in any way limiting its scope.

### EXAMPLES

The following materials and methods were employed in the experiments described in Examples 1-9.

RNA Sequencing

RNA sequencing with Illumina methods was performed. One microgram of total RNA was used as the input to an mRNA capture with oligo-dT coated magnetic beads. The mRNA was fragmented, and then a random-primed cDNA synthesis was performed. The resulting double-strand cDNA was used as the input to a standard Illumina (San Diego, CA, USA) library prep with end-repair, adapter ligation with unique indexed barcode and PCR amplification performed to give a sequencing-ready library. The final purified product was quantitated by qPCR before cluster generation and sequencing.

Single-molecule real-time RNA sequencing was performed. Full-length cDNA was synthesized and amplified using the NEBNext© Single Cell/Low Input cDNA Synthesis & Amplification Module (New England Biolabs, Ipswich, MA, USA) and the Iso-Seq Express Oligo Kit (Pacific Biosciences, Menlo Park, CA, USA). cDNA was amplified with 12 PCR cycles and size selected using 0.84×ProNex beads (Promega, Madison, WI, USA). SMRTbell libraries were then prepared using the SMRTbell Express Template Prep Kit 2.0 (Pacific Biosciences). Transcripts above 2.5 kb were selected. Sequencing primer v4 was annealed and Sequel II polymerase 2.0 was bound to libraries prior to loading each on one 8M SMRT Cell on the Sequel II System using diffusion loading. Sequencing was performed with 2h pre-extension and a 24 h movie.

Cell Lines

K562 cells were transduced as previously described to express CD19 (CD19-K562) or low-affinity nerve growth factor (NFGR-K562) (Kochenderfer et al., Journal of Immunotherapy, 32: 689-702 (2009), incorporated by reference herein). K562 cells were also transduced to express CD20. The genes were transferred to K562 cells by standard methods with the MSGV1 gamma-retroviral vector. The NGFR-K562 cells served as CD19-negative control cells. CCRF-CEM cells (ATCC, Manassas, VA, USA) also served as negative control cells. CD19⁺ NALM6 cells (acute lymphoid leukemia from DSMZ, Braunschweig, Germany) were also used. Toledo and st486 were CD19⁺ and CD20⁺ lymphoma cell lines obtained from ATCC. Toledo CD19 −/−and st486 CD19 −/−cell lines were both produced by clustered regularly interspaced short palindromic repeats (CRISPR)/CRISPR-associated protein (Cas)9 knockout of CD19 from parent cell lines.

Design and Construction of Bicistronic CARs Targeting CD19 and CD20

A fully-human anti-CD19 CAR designated Hul9-CD828Z as previously designed (Alabanza et al., Molecular Therapy, 25: 2452-2465 (2017), incorporated by reference herein) was used as the basis for the anti-CD19 CAR. A scFv designated Hul9 was designed with the following sequence from 5′ to 3′: Human CD8α signal sequence, light chain variable region, a linker peptide (GSTSGSGKPGSGEGSTKG, SEQ ID NO: 29), heavy chain variable region. A DNA sequence encoding a CAR with the following components from 5′ to 3′ was designed: Hul9 scFv, part of the extracellular region and the transmembrane region of the human CD8a molecule, the cytoplasmic portion of the human CD28 molecule, and the cytoplasmic part of the human CD3ζ molecule.

To form a construct with the ability to recognize both CD19 and CD20, Hul9-CD828Z was incorporated into bicistronic constructs also encoding a separate CAR targeting CD20. From N-terminus to C-terminus, the first bicistronic construct included the CD8α signal sequence followed by the Hul9-CD828Z CAR sequence as described above. Next, a furin cleavage site with the amino acid sequence RAKR (SEQ ID NO: 38), a spacer with the amino acid sequence SGSGAP (SEQ ID NO: 39), and an F2A ribosomal skip cleavage sequence with an amino acid sequence of VKQTLNFDLLKLAGDVESNPGP (SEQ ID NO: 40) from the foot-and-mouth disease virus was incorporated. Following the F2A sequence, an anti-CD20 CAR designated Hu20-CD8BBZ was incorporated. Hu20-CD8BBZ has a granulocyte-macrophage colony stimulating receptor (GM-CSFr) signal sequence followed by an scFv designated Hu20. The variable regions of the Hu20 scFv are from an antibody called 2.1.2 (WO 2006/130458, incorporated by reference herein). The light chain and heavy chain variable domains of the Hu20 scFv were connected by a (G4S)3 linker with the amino acid sequence GGGGSGGGGSGGGGS (SEQ ID NO: 10). The light chain variable region comes first in this scFv followed by the linker and the heavy chain variable region. After the scFv, a CD8α hinge and transmembrane region was added followed by the cytoplasmic region of human 4-1BB and then the cytoplasmic region of human CD3ζ. This entire CAR construct including the Hul9-CD828Z CAR, the intervening F2A-containing sequence, and the Hu20-CD8BBZ CAR was designated Hu1928-Hu20BB (“Hu1928-Hu20BB-original”). The DNA sequence encoding Hu1928-Hu20BB was synthesized and cloned into the MSGV1 gamma-retroviral backbone by standard methods (Hughes et al., Human Gene Therapy, 16:457-72 (2005), incorporated by reference herein).

To prevent retroviral recombination events that are driven by annealing of regions of identical nucleotide sequence, the DNA sequence of Hu1928-Hu20BB-original was designed to eliminate as much as possible areas of identical DNA sequence in different parts of the construct. As a first step, the CD8α signal sequence of the Hu20-CD8BBZ CAR was replaced with the signal sequence of the granulocyte-macrophage colony stimulating factor receptor (GM-CSFr) that was used in a previous CAR (Kochenderfer et al., Journal of Immunotherapy, 32:689-702 (2009), incorporated by reference herein). Next, the regions of Hu1928-Hu20BB-original from the CD8α signal sequence of the Hul9-CD828Z CAR to the CD3ζ domain of the Hu20-CD8BBZ CAR were assessed for areas of DNA sequence that were identical between the two CARs making up the construct. Areas of identical sequence in proteins of the same type in both CARs were eliminated. For example, the light chain variable region domain of Hul9-CD828Z was compared to the light chain variable region of Hu20-CD8BBZ. When an area of identical DNA sequence shared by the two CARs was identified, a change was made in the DNA sequence of a DNA triplet codon by substituting an alternate DNA triplet codon encoding the same amino acid. The GenScript Codon Usage Frequency Table (www.genscript.com/tools/codon-frequency-table) was used to do this. This process was performed for the light chain variable domains, scFv linkers, heavy chain variable domains, CD8α hinge and transmembrane domains, and CD3ζ domains of Hul9-CD828Z and Hu20-CD8BBZ of the Hu1928-Hu20BB-original construct. An iterative process of designing 7 new Hu1928-Hu20BB DNA sequences with ever increasing changes in the DNA sequence was performed. With each new design, the Hu1928-Hu20BB sequence was synthesized and cloned into the MSGV1 vector by standard methods (Kochenderfer et al., Journal of Immunotherapy, 32:689-702 (2009) and Hughes et al., Human Gene Therapy, 16:457-72 (2005), each of which is incorporated by reference herein). In synthesizing these new versions, new DNA fragments were synthesized by Thermo/GeneArt with restriction sites at the 5′ and 3′ ends. These new DNA fragments were then ligated into one of the earlier versions of the MSGV1-Hu1928-Hu20BB plasmid. For the first of the 7 new Hu1928-Hu20BB versions, the new DNA fragment was used to replace the corresponding region in MSGV1-Hu1928-Hu20BB-original. Subsequently, new DNA fragments for each subsequent version were used to replace a corresponding region in a prior version of Hu1928-Hu20BB. This replacement was performed by restriction enzyme digestion (enzymes from New England Biolabs) followed by ligation of the new fragment into the restriction-enzyme-digested prior MSGV1-Hu1928-Hu20BB version (Roche (Basel, Switzerland) DNA ligation kit).

Human T cells were then transduced with transiently-produced gamma-retroviral vector encoding each new Hu19280-Hu20BB design to assess for cell-surface expression of these CARs. This was done because changes in the DNA sequence might have caused a decrease in expression. During these iterative changes in DNA sequence and expression testing, it was found that expression of both Hul9-CD828Z and Hu20-CD8BBZ increased. Further changes in the new designs were made. This design was designated Hu1928-Hu20BB standard (std) 10-5-2020.

Three more bicistronic CAR constructs were designed and synthesized. The sequences of these CAR constructs started with a CD8α signal sequence that was followed by the Hul9-CD828Z CAR as described above. After the Hul9-CD828Z component, the sequence included the same furin binding site plus spacer plus F2A sequence as Hu1928-Hu20BB std 10-5-2020. Following the F2A-containing region, each of the 3 novel CAR constructs contained a different CAR that incorporated the Hu20 scFv.

Hu1928-Hu20BB long 10-21-2020 has the same nucleotide sequence as Hu1928-Hu20BB std 10-5-2020 except that the linker of the Hu20 scFv has been lengthened to GGGGSGGGGSGGGGSGGGGS (SEQ ID NO: 41) (G4S)4 rather than the SEQ ID NO: 10 of the Hu1928-Hu20BB std 10-5-2020 construct. The MSGV1-Hu1928-Hu20BB long 10-21-2020 plasmid was generated by replacing the region of the Hu20 scFv in the MSGV1-Hu1928-Hu20BB std 10-5-2020 with a newly synthesized DNA fragment (Thermo/GeneArt) by standard recombinant DNA methods.

Hu1928-Hu2028 long has the same nucleotide sequence as Hu1928-Hu20BB long 10-21-2020 except that the 4-1BB domain of the Hu20-containing CAR has been replaced with the cytoplasmic sequence of CD28. This was performed by synthesizing a DNA fragment containing the cytoplasmic domain of human CD28 (Thermo/GeneArt). This fragment was used to replace the region of the MSGV1-Hu1928-Hu20BB long 10-21-2020 plasmid containing the 4-1BB domain by standard recombinant DNA methods.

Hu1928-Hu2028 std (standard) has the same nucleotide sequence as Hu1928-Hu2028 long except that the amino acid sequence of the linker of the Hu20-containing CAR has been shortened from (G4S)4 in the long version to (G4S)3 in the std version. This CAR was constructed by replacing the region of the Hu20 scFv in the MSGV1-Hu1928-Hu2028 long plasmid with a new DNA fragment (Thermo/GeneArt) encoding the shorter (G4S)3 linker.

A series of plasmids were generated encoding each unique monospecific CAR contained in the bicistronic constructs described above. These CARs all had the new nucleotide regions as in the bicistronic constructs. The new monocistronic constructs were: 10-5-2020 Hul9-CD828Z, 9-15-2020 Hu20-CD8BBZ std, Hu20-CD828Z std, Hu20-CD8BBZ long, Hu20-CD828Z long. The new versions of previously-reported CARs have new DNA sequences to reduce the risk of recombination. MSGV1 plasmids encoding all 5 of these CARs were constructed by synthesizing DNA fragments (Thermo/GeneArt) encoding the CARs or portions of the CARs with restriction endonuclease sites at each end and ligating these fragments into the appropriate MSGV1 plasmids by standard techniques. MSGV1-SP6-CD828Z encodes a previously-described negative control CAR.

The full-length sequences of the bicistronic and monospecific constructs described above are:
Hu1928-Hu20BB-original (SEQ ID NO: 46, amino acid; SEQ ID NO: 47, DNA)
Hu1928-Hu20BB standard (std) 10-5-2020 (SEQ ID NO: 71, amino acid; SEQ ID NO: 42, DNA)
Hu1928-Hu20BB long 10-21-2020 (SEQ ID NO: 72, amino acid; SEQ ID NO: 43, DNA)
Hu1928-Hu2028 long (SEQ ID NO: 73, amino acid; SEQ ID NO: 44, DNA)
Hu1928-Hu2028 std (standard) (SEQ ID NO: 74, amino acid; SEQ ID NO: 45, DNA)
10-5-2020 Hul9-CD828Z (SEQ ID NO: 75, amino acid; SEQ ID NO: 48, DNA)
9-15-2020 Hu20-CD8BBZ std (SEQ ID NO: 76, amino acid; SEQ ID NO: 49, DNA)
Hu20-CD828Z std (SEQ ID NO: 77, amino acid; SEQ ID NO: 50, DNA)
Hu20-CD8BBZ long (SEQ ID NO: 78, amino acid; SEQ ID NO: 51, DNA)
Hu20-CD828Z long (SEQ ID NO: 79, amino acid; SEQ ID NO: 52, DNA)

The following tables present the full-length DNA sequences, depicting the domains within the full-length sequences.

TABLE 1
Hu1928-Hu20BB standard (std) 10-5-2020

| | SEQ ID | |
|---|---|---|
| Description | NO: | Sequence |
| CD8α SS | 53 | ATGGCCCTGCCTGTGACAGC |
| | | TCTGCTGCTGCCCCTGGCCC |
| | | TGCTGCTGCATGCCGCCAGA |
| | | CCT |
| | 30 | MALPVTALLLPLALLLHAARP |
| CD19 scFv: | | |
| 47G4 LC | 54 | GAGATCGTGCTGACCCAGTC |
| | | TCCCGGTACCCTGTCTCTCA |
| | | GCCCAGGAGAGAGAGCCACC |
| | | CTGAGCTGCAGAGCCAGCCA |
| | | GAGCGTGTCCAGCAGCTACC |
| | | TGGCCTGGTATCAGCAGAAG |
| | | CCCGGACAGGCCCCCAGACT |
| | | GCTGATCTACGGCGCCAGCT |
| | | CTAGAGCCACCGGCATCCCC |
| | | GACAGATTCAGCGGCAGCGG |
| | | CAGTGGTACCGACTTCACCC |
| | | TGACCATCAGCAGACTGGAA |
| | | CCCGAGGACTTCGCCGTGTA |
| | | TTACTGCCAGCAGTACGGCA |
| | | GCAGCCGGTTCACCTTCGGC |
| | | CCTGGCACCAAGGTGGACAT |
| | | CAAG |
| | 6 | EIVLTQSPGTLSLSPGERAT |
| | | LSCRASQSVSSSYLAWYQQK |
| | | PGQAPRLLIYGASSRATGIP |
| | | DRFSGSGSGTDFTLTISRLE |
| | | PEDFAVYYCQQYGSSRFTFG |
| | | PGTKVDIK |
| Linker | 55 | GGCAGCACCTCCGGCAGCGG |
| | | CAAGCCTGGCTCTGGCGAGG |
| | | GCTCTACCAAGGGC |
| | 29 | GSTSGSGKPGSGEGSTKG |
| 47G4 HC | 56 | CAGGTGCAGCTGGTGCAGTC |
| | | TGGCGCCGAAGTCAAGAAAC |
| | | CCGGCTCTAGCGTGAAGGTG |
| | | TCCTGCAAGGACAGCGGCGG |
| | | CACCTTCAGCAGCTACGCCA |
| | | TCAGCTGGGTGCGCCAGGCC |
| | | CCAGGACAGGGGCTGGAATG |
| | | GATGGGCGGCATCATCCCCA |
| | | TCTTCGGCACCACCAACTAC |
| | | GCCCAGCAGTTCCAGGGCAG |
| | | AGTGACCATCACCGCCGACG |
| | | AGAGCACCAGCACCGCCTAC |
| | | ATGGAACTGAGCAGCCTGCG |
| | | GAGCGAGGACACAGCCGTGT |
| | | ATTACTGTGCCCGCGAGGCC |
| | | GTGGCCGCCGACTGGCTGGA |
| | | TCCTTGGGGACAGGGCACCC |
| | | TGGTGACAGTGTCCAGC |
| | 5 | QVQLVQSGAEVKKPGSSVKV |
| | | SCKDSGGTFSSYAISWVRQA |
| | | PGQGLEWMGGIIPIFGTTNY |
| | | AQQFQGRVTITADESTSTAY |
| | | MELSSLRSEDTAVYYCAREA |
| | | VAADWLDPWGQGTLVTVSS |
| CD8α | 57 | TTCGTGCCAGTGTTTCTACC |
| | | TGCCAAGCCGACCACCACGC |
| | | CTGCCCCTAGACCTCCTACA |
| | | CCCGCCCCTACAATCGCCAG |
| | | CCAGCCTCTGTCTCTGAGGC |
| | | CCGAGGCTTGTAGACCTGCT |
| | | GCTGGCGGAGCCGTGCACAC |
| | | CAGAGGACTGGATTTCGCCT |
| | | GCGACATCTACATCTGGGCC |
| | | CCTCTGGCCGGCACATGTGG |
| | | CGTGCTGCTGCTCAGCCTGG |
| | | TCATCACCCTGTACTGTAAC |
| | | CACCGGAAC |
| | 31 | FVPVFLPAKPTTTPAPRPPT |
| | | PAPTIASQPLSLRPEACRPA |
| | | AGGAVHTRGLDFACDIYIWA |
| | | PLAGTCGVLLLSLVITLYCN |
| | | HRN |
| CD28 | 58 | AGAAGCAAGCGGAGCAGACT |
| | | GCTGCACAGCGACTACATGA |
| | | ACATGACCCCTAGACGGCCC |
| | | GGACCTACCAGAAAGCACTA |
| | | CCAGCCTTACGCTCCTCCTC |
| | | GGGACTTTGCCGCCTATCGG |
| | | AGC |
| | 32 | RSKRSRLLHSDYMNMTPRRP |
| | | GPTRKHYQPYAPPRDFAAYR |
| | | S |
| CD3ζ | 59 | AGAGTGAAGTTCAGCAGATC |
| | | AGCCGATGCTCCTGCCTACC |
| | | AGCAGGGCCAGAATCAGCTG |
| | | TACAACGAGCTGAACCTGGG |
| | | GAGAAGAGAAGAGTACGACG |
| | | TGCTGGATAAGCGGAGAGGC |
| | | AGAGATCCTGAGATGGGCGG |
| | | CAAGCCCAGACGGAAGAATC |
| | | CTCAGGAGGGCCTGTATAAT |
| | | GAGCTGCAGAAAGACAAGAT |
| | | GGCCGAGGCCTACAGCGAGA |
| | | TCGGCATGAAAGGCGAGAGA |
| | | AGAAGAGGCAAGGGCCACGA |
| | | TGGACTGTACCAGGGACTGA |
| | | GCACAGCCACCAAGGATACC |
| | | TACGATGCCCTGCACATGCA |
| | | GGCCCTTCCACCTAGA |
| | 33 | RVKFSRSADAPAYQQGQNQL |
| | | YNELNLGRREEYDVLDKRRG |
| | | RDPEMGGKPRRKNPQEGLYN |
| | | ELQKDKMAEAYSEIGMKGER |
| | | RRGKGHDGLYQGLSTATKDT |
| | | YDALHMQALPPR |
| Cleavage | 60 | AGGGCCAAGAGATCTGGATC |
| sequence | | TGGCGCCCCTGTGAAGCAGA |
| | | CCCTGAATTTCGACCTGCTG |
| | | AAGCTGGCCGGCGACGTGGA |
| | | ATCTAATCCTGGACCT |
| | 37 | RAKRSGSGAPVKQTLNFDLL |
| | | KLAGDVESNPGP |
| GM-CSF SS | 61 | ATGCTTCTCCTGGTGACAAG |
| | | CCTTCTGCTCTGTGAGTTAC |
| | | CACACCCAGCATTCCTCCTG |
| | | ATCCCA |
| | 80 | MLLLVTSLLLCELPHPAFLL |
| | | IP |
| CD20 scFv: | | |
| 2.1.2 LC | 62 | GATATCGTGATGACACAGAC |
| | | ACCTCACAGCAGCCCTGTTA |
| | | CACTGGGACAGCCTGCCAGC |
| | | ATCTCCTGTAGAAGCTCCCA |
| | | GAGCCTGGTGTCCAGAGATG |
| | | GCAATACCTACCTGAGCTGG |
| | | CTGCAGCAGAGGCCTGGACA |
| | | ACCTCCTAGGCTGCTGATTT |
| | | ACAAGATCAGCAACCGGTTC |
| | | AGCGGCGTGCCCAATAGATT |
| | | TTCTGGAAGCGGAGCCGGCA |
| | | CAGACTTTACCCTGAAGATT |
| | | TCTAGAGTGAAGGCCGAGGA |
| | | CGTGGGCGTGTACTACTGTA |
| | | TGCAGGCCACACAGTTCCCT |
| | | CTGACCTTTGGCCAGGGCAC |
| | | CAGACTGGAAATCAAA |
| | 14 | DIVMTQTPHSSPVTLGQPAS |
| | | ISCRSSQSLVSRDGNTYLSW |
| | | LQQRPGQPPRLLIYKISNRF |
| | | SGVPNRFSGSGAGTDFTLKI |
| | | SRVKAEDVGVYYCMQATQFP |
| | | LTFGQGTRLEIK |
| Linker | 63 | GGTGGCGGAGGTTCCGGCGG |
| | | CGGAGGATCAGGCGGAGGTG |
| | | GAAGT |
| | 10 | GGGGSGGGGSGGGGS |
| 2.1.2 HC | 64 | GAAGTCCAGCTCGTTCAGTC |
| | | CGGAGCCGAGGTGAAGAAGC |
| | | CTGGCGAGTCTCTGAAGATC |
| | | AGCTGCAAAGGCAGCGGCTA |
| | | CAGCTTCACCAGCTATTGGA |
| | | TCGGCTGGGTCCGACAGATG |
| | | CCTGGCAAAGGACTGGAGTG |
| | | GATGGGCATCATCTACCCCG |
| | | GCGACAGCGATACCAGATAC |
| | | AGCCCTAGCTTTCAGGGCCA |
| | | AGTGACCATCAGCGCCGACA |
| | | AGAGCATCAGCACAGCCTAC |
| | | CTGCAGTGGTCTAGCCTGAA |
| | | GGCCAGCGACACCGCCATGT |
| | | ACTATTGTGCCAGACAGGGC |
| | | GACTTTTGGAGCGGCTATGG |
| | | TGGCATGGATGTGTGGGGCC |
| | | AGGGCACAACAGTGACCGTG |
| | | TCTAGC |
| | 13 | EVQLVQSGAEVKKPGESLKI |
| | | SCKGSGYSFTSYWIGWVRQM |
| | | PGKGLEWMGIIYPGDSDTRY |
| | | SPSFQGQVTISADKSISTAY |
| | | LQWSSLKASDTAMYYCARQG |
| | | DFWSGYGGMDVWGQGTTVTV |
| | | SS |
| CD8α | 65 | TTCGTTCCGGTTTTTCTGCC |
| | | GGCAAAGCCTACAACTACCC |
| | | CCGCACCCCGGCCCCCAACT |
| | | CCCGCTCCAACGATCGCATC |
| | | ACAACCACTTTCACTCCGAC |
| | | CAGAGGCTTGCAGACCGGCT |
| | | GCGGGAGGCGCGGTACACAC |
| | | GCGGGGGCTCGATTTTGCTT |
| | | GCGATATTTACATTTGGGCT |
| | | CCTCTTGCCGGTACATGCGG |
| | | TGTCTTGCTCCTGTCCCTCG |
| | | TCATTACTCTCTATTGCAAC |
| | | CATAGGAAC |
| | 31 | FVPVFLPAKPTTTPAPRPPT |
| | | PAPTIASQPLSLRPEACRPA |
| | | AGGAVHTRGLDFACDIYIWA |
| | | PLAGTCGVLLLSLVITLYCN |
| | | HRN |
| 4-1BB | 66 | AAGCGAGGCCGGAAGAAGCT |
| | | GCTGTACATCTTCAAGCAGC |
| | | CTTTCATGCGGCCCGTGCAG |
| | | ACCACACAAGAGGAAGATGG |
| | | CTGTAGCTGCAGATTCCCCG |
| | | AGGAAGAAGAAGGCGGCTGC |
| | | GAGCTG |
| | 34 | KRGRKKLLYIFKQPFMRPVQ |
| | | TTQEEDGCSCRFPEEEEGGC |
| | | EL |
| CD3ζ | 67 | AGGGTGAAATTCTCTAGAAG |
| | | CGCCGACGCACCCGCATATC |
| | | AGCAAGGACAAAACCAGCTC |
| | | TATAACGAACTCAACCTCGG |
| | | CAGACGCGAGGAATATGATG |
| | | TGCTGGACAAGAGGCGGGGA |
| | | CGCGATCCAGAAATGGGAGG |
| | | AAAGCCTCGGAGAAAGAACC |
| | | CACAAGAGGGACTTTACAAC |
| | | GAACTCCAAAAGGATAAGAT |
| | | GGCAGAAGCCTATTCCGAGA |
| | | TTGGAATGAAGGGCGAACGT |
| | | CGGAGAGGAAAGGGACACGA |
| | | CGGCCTTTATCAGGGCCTGT |
| | | CCACCGCCACAAAAGATACG |
| | | TATGACGCTCTCCACATGCA |
| | | AGCGTTGCCCCCCCGC |
| | 33 | RVKFSRSADAPAYQQGQNQL |
| | | YNELNLGRREEYDVLDKRRG |
| | | RDPEMGGKPRRKNPQEGLYN |
| | | ELQKDKMAEAYSEIGMKGER |
| | | RRGKGHDGLYQGLSTATKDT |
| | | YDALHMQALPPR |

TABLE 2
Hu1928-Hu20BB long 10-21-2020

| | SEQ ID | |
|---|---|---|
| Description | NO: | Sequence |
| CD8α SS | 53 | ATGGCCCTGCCTGTGACAGCTCTGC |
| | | TGCTGCCCCTGGCCCTGCTGCTGCA |
| | | TGCCGCCAGACCT |
| | 30 | MALPVTALLLPLALLLHAARP |
| CD19 scFv: | | |
| 47G4 LC | 54 | GAGATCGTGCTGACCCAGTCTCCCG |
| | | GTACCCTGTCTCTCAGCCCAGGAGA |
| | | GAGAGCCACCCTGAGCTGCAGAGCC |
| | | AGCCAGAGCGTGTCCAGCAGCTACC |
| | | TGGCCTGGTATCAGCAGAAGCCCGG |
| | | ACAGGCCCCCAGACTGCTGATCTAC |
| | | GGCGCCAGCTCTAGAGCCACCGGCA |
| | | TCCCCGACAGATTCAGCGGCAGCGG |
| | | CAGTGGTACCGACTTCACCCTGACC |
| | | ATCAGCAGACTGGAACCCGAGGACT |
| | | TCGCCGTGTATTACTGCCAGCAGTA |
| | | CGGCAGCAGCCGGTTCACCTTCGGC |
| | | CCTGGCACCAAGGTGGACATCAAG |
| | 6 | EIVLTQSPGTLSLSPGERATLSCRA |
| | | SQSVSSSYLAWYQQKPGQAPRLLIY |
| | | GASSRATGIPDRFSGSGSGTDFTLT |
| | | ISRLEPEDFAVYYCQQYGSSRFTFG |
| | | PGTKVDIK |
| Linker | 55 | GGCAGCACCTCCGGCAGCGGCAAGC |
| | | CTGGCTCTGGCGAGGGCTCTACCAA |
| | | GGGC |
| | 29 | GSTSGSGKPGSGEGSTKG |
| 47G4 HC | 56 | CAGGTGCAGCTGGTGCAGTCTGGCG |
| | | CCGAAGTCAAGAAACCCGGCTCTAG |
| | | CGTGAAGGTGTCCTGCAAGGACAGC |
| | | GGCGGCACCTTCAGCAGCTACGCCA |
| | | TCAGCTGGGTGCGCCAGGCCCCAGG |
| | | ACAGGGGCTGGAATGGATGGGCGGC |
| | | ATCATCCCCATCTTCGGCACCACCA |
| | | ACTACGCCCAGCAGTTCCAGGGCAG |
| | | AGTGACCATCACCGCCGACGAGAGC |
| | | ACCAGCACCGCCTACATGGAACTGA |
| | | GCAGCCTGCGGAGCGAGGACACAGC |
| | | CGTGTATTACTGTGCCCGCGAGGCC |
| | | GTGGCCGCCGACTGGCTGGATCCTT |
| | | GGGGACAGGGCACCCTGGTGACAGT |
| | | GTCCAGC |
| | 5 | QVQLVQSGAEVKKPGSSVKVSCKDS |
| | | GGTFSSYAISWVRQAPGQGLEWMGG |
| | | IIPIFGTTNYAQQFQGRVTITADES |
| | | TSTAYMELSSLRSEDTAVYYCAREA |
| | | VAADWLDPWGQGTLVTVSS |
| CD8α | 57 | TTCGTGCCAGTGTTTCTACCTGCCA |
| | | AGCCGACCACCACGCCTGCCCCTAG |
| | | ACCTCCTACACCCGCCCCTACAATC |
| | | GCCAGCCAGCCTCTGTCTCTGAGGC |
| | | CCGAGGCTTGTAGACCTGCTGCTGG |
| | | CGGAGCCGTGCACACCAGAGGACTG |
| | | GATTTCGCCTGCGACATCTACATCT |
| | | GGGCCCCTCTGGCCGGCACATGTGG |
| | | CGTGCTGCTGCTCAGCCTGGTCATC |
| | | ACCCTGTACTGTAACCACCGGAAC |
| | 31 | FVPVFLPAKPTTTPAPRPPTPAPTI |
| | | ASQPLSLRPEACRPAAGGAVHTRGL |
| | | DFACDIYIWAPLAGTCGVLLLSLVI |
| | | TLYCNHRN |
| CD28 | 58 | AGAAGCAAGCGGAGCAGACTGCTGC |
| | | ACAGCGACTACATGAACATGACCCC |
| | | TAGACGGCCCGGACCTACCAGAAAG |
| | | CACTACCAGCCTTACGCTCCTCCTC |
| | | GGGACTTTGCCGCCTATCGGAGC |
| | 32 | RSKRSRLLHSDYMNMTPRRPGPTRK |
| | | HYQPYAPPRDFAAYRS |
| CD3ζ | 59 | AGAGTGAAGTTCAGCAGATCAGCCG |
| | | ATGCTCCTGCCTACCAGCAGGGCCA |
| | | GAATCAGCTGTACAACGAGCTGAAC |
| | | CTGGGGAGAAGAGAAGAGTACGACG |
| | | TGCTGGATAAGCGGAGAGGCAGAGA |
| | | TCCTGAGATGGGCGGCAAGCCCAGA |
| | | CGGAAGAATCCTCAGGAGGGCCTGT |
| | | ATAATGAGCTGCAGAAAGACAAGAT |
| | | GGCCGAGGCCTACAGCGAGATCGGC |
| | | ATGAAAGGCGAGAGAAGAAGAGGCA |
| | | AGGGCCACGATGGACTGTACCAGGG |
| | | ACTGAGCACAGCCACCAAGGATACC |
| | | TACGATGCCCTGCACATGCAGGCCC |
| | | TTCCACCTAGA |
| | 33 | RVKFSRSADAPAYQQGQNQLYNELN |
| | | LGRREEYDVLDKRRGRDPEMGGKPR |
| | | RKNPQEGLYNELQKDKMAEAYSEIG |
| | | MKGERRRGKGHDGLYQGLSTATKDT |
| | | YDALHMQALPPR |
| Cleavage | 60 | AGGGCCAAGAGATCTGGATCTGGCG |
| sequence | | CCCCTGTGAAGCAGACCCTGAATTT |
| | | CGACCTGCTGAAGCTGGCCGGCGAC |
| | | GTGGAATCTAATCCTGGACCT |
| | 37 | RAKRSGSGAPVKQTLNFDLLKLAGD |
| | | VESNPGP |
| GM-CSF SS | 61 | ATGCTTCTCCTGGTGACAAGCCTTC |
| | | TGCTCTGTGAGTTACCACACCCAGC |
| | | ATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTC |
| | | ACAGCAGCCCTGTTACACTGGGACA |
| | | GCCTGCCAGCATCTCCTGTAGAAGC |
| | | TCCCAGAGCCTGGTGTCCAGAGATG |
| | | GCAATACCTACCTGAGCTGGCTGCA |
| | | GCAGAGGCCTGGACAACCTCCTAGG |
| | | CTGCTGATTTACAAGATCAGCAACC |
| | | GGTTCAGCGGCGTGCCCAATAGATT |
| | | TTCTGGAAGCGGAGCCGGCACAGAC |
| | | TTTACCCTGAAGATTTCTAGAGTGA |
| | | AGGCCGAGGACGTGGGCGTGTACTA |
| | | CTGTATGCAGGCCACACAGTTCCCT |
| | | CTGACCTTTGGCCAGGGCACCAGAC |
| | | TGGAAATCAAA |
| | 14 | DIVMTQTPHSSPVTLGQPASISCRS |
| | | SQSLVSRDGNTYLSWLQQRPGQPPR |
| | | LLIYKISNRFSGVPNRFSGSGAGTD |
| | | FTLKISRVKAEDVGVYYCMQATQFP |
| | | LTFGQGTRLEIK |
| Linker | 68 | GGAGGAGGCGGGAGTGGTGGCGGAG |
| | | GTTCCGGCGGCGGAGGATCAGGCGG |
| | | AGGTGGAAGT |
| | 41 | GGGGSGGGGSGGGGSGGGGS |
| 2.1.2 HC | 64 | GAAGTCCAGCTCGTTCAGTCCGGAG |
| | | CCGAGGTGAAGAAGCCTGGCGAGTC |
| | | TCTGAAGATCAGCTGCAAAGGCAGC |
| | | GGCTACAGCTTCACCAGCTATTGGA |
| | | TCGGCTGGGTCCGACAGATGCCTGG |
| | | CAAAGGACTGGAGTGGATGGGCATC |
| | | ATCTACCCCGGCGACAGCGATACCA |
| | | GATACAGCCCTAGCTTTCAGGGCCA |
| | | AGTGACCATCAGCGCCGACAAGAGC |
| | | ATCAGCACAGCCTACCTGCAGTGGT |
| | | CTAGCCTGAAGGCCAGCGACACCGC |
| | | CATGTACTATTGTGCCAGACAGGGC |
| | | GACTTTTGGAGCGGCTATGGTGGCA |
| | | TGGATGTGTGGGGCCAGGGCACAAC |
| | | AGTGACCGTGTCTAGC |
| | 13 | EVQLVQSGAEVKKPGESLKISCKGS |
| | | GYSFTSYWIGWVRQMPGKGLEWMGI |
| | | IYPGDSDTRYSPSFQGQVTISADKS |
| | | ISTAYLQWSSLKASDTAMYYCARQG |
| | | DFWSGYGGMDVWGQGTTVTVSS |
| CD8α | 65 | TTCGTTCCGGTTTTTCTGCCGGCAA |
| | | AGCCTACAACTACCCCCGCACCCCG |
| | | GCCCCCAACTCCCGCTCCAACGATC |
| | | GCATCACAACCACTTTCACTCCGAC |
| | | CAGAGGCTTGCAGACCGGCTGCGGG |
| | | AGGCGCGGTACACACGCGGGGGCTC |
| | | GATTTTGCTTGCGATATTTACATTT |
| | | GGGCTCCTCTTGCCGGTACATGCGG |
| | | TGTCTTGCTCCTGTCCCTCGTCATT |
| | | ACTCTCTATTGCAACCATAGGAAC |
| | 31 | FVPVFLPAKPTTTPAPRPPTPAPTI |
| | | ASQPLSLRPEACRPAAGGAVHTRGL |
| | | DFACDIYIWAPLAGTCGVLLLSLVI |
| | | TLYCNHRN |
| 4-1BB | 66 | AAGCGAGGCCGGAAGAAGCTGCTGT |
| | | ACATCTTCAAGCAGCCTTTCATGCG |
| | | GCCCGTGCAGACCACACAAGAGGAA |
| | | GATGGCTGTAGCTGCAGATTCCCCG |
| | | AGGAAGAAGAAGGCGGCTGCGAGCT |
| | | G |
| | 34 | KRGRKKLLYIFKQPFMRPVQTTQEE |
| | | DGCSCRFPEEEEGGCEL |
| CD3ζ | 67 | AGGGTGAAATTCTCTAGAAGCGCCG |
| | | ACGCACCCGCATATCAGCAAGGACA |
| | | AAACCAGCTCTATAACGAACTCAAC |
| | | CTCGGCAGACGCGAGGAATATGATG |
| | | TGCTGGACAAGAGGCGGGGACGCGA |
| | | TCCAGAAATGGGAGGAAAGCCTCGG |
| | | AGAAAGAACCCACAAGAGGGACTTT |
| | | ACAACGAACTCCAAAAGGATAAGAT |
| | | GGCAGAAGCCTATTCCGAGATTGGA |
| | | ATGAAGGGCGAACGTCGGAGAGGAA |
| | | AGGGACACGACGGCCTTTATCAGGG |
| | | CCTGTCCACCGCCACAAAAGATACG |
| | | TATGACGCTCTCCACATGCAAGCGT |
| | | TGCCCCCCCGC |
| | 33 | RVKFSRSADAPAYQQGQNQLYNELN |
| | | LGRREEYDVLDKRRGRDPEMGGKPR |
| | | RKNPQEGLYNELQKDKMAEAYSEIG |
| | | MKGERRRGKGHDGLYQGLSTATKDT |
| | | YDALHMQALPPR |

TABLE 3
Hu1928-Hu2028 long

| | SEQ ID | |
|---|---|---|
| Description | NO: | Sequence |
| CD8α SS | 53 | ATGGCCCTGCCTGTGACAGCTCTGC |
| | | TGCTGCCCCTGGCCCTGCTGCTGCA |
| | | TGCCGCCAGACCT |
| | 30 | MALPVTALLLPLALLLHAARP |
| CD19 scFv: | | |
| 47G4 LC | 54 | GAGATCGTGCTGACCCAGTCTCCCG |
| | | GTACCCTGTCTCTCAGCCCAGGAGA |
| | | GAGAGCCACCCTGAGCTGCAGAGCC |
| | | AGCCAGAGCGTGTCCAGCAGCTACC |
| | | TGGCCTGGTATCAGCAGAAGCCCGG |
| | | ACAGGCCCCCAGACTGCTGATCTAC |
| | | GGCGCCAGCTCTAGAGCCACCGGCA |
| | | TCCCCGACAGATTCAGCGGCAGCGG |
| | | CAGTGGTACCGACTTCACCCTGACC |
| | | ATCAGCAGACTGGAACCCGAGGACT |
| | | TCGCCGTGTATTACTGCCAGCAGTA |
| | | CGGCAGCAGCCGGTTCACCTTCGGC |
| | | CCTGGCACCAAGGTGGACATCAAG |
| | 6 | EIVLTQSPGTLSLSPGERATLSCRA |
| | | SQSVSSSYLAWYQQKPGQAPRLLIY |
| | | GASSRATGIPDRFSGSGSGTDFTLT |
| | | ISRLEPEDFAVYYCQQYGSSRFTFG |
| | | PGTKVDIK |
| Linker | 55 | GGCAGCACCTCCGGCAGCGGCAAGC |
| | | CTGGCTCTGGCGAGGGCTCTACCAA |
| | | GGGC |
| | 29 | GSTSGSGKPGSGEGSTKG |
| 47G4 HC | 56 | CAGGTGCAGCTGGTGCAGTCTGGCG |
| | | CCGAAGTCAAGAAACCCGGCTCTAG |
| | | CGTGAAGGTGTCCTGCAAGGACAGC |
| | | GGCGGCACCTTCAGCAGCTACGCCA |
| | | TCAGCTGGGTGCGCCAGGCCCCAGG |
| | | ACAGGGGCTGGAATGGATGGGCGGC |
| | | ATCATCCCCATCTTCGGCACCACCA |
| | | ACTACGCCCAGCAGTTCCAGGGCAG |
| | | AGTGACCATCACCGCCGACGAGAGC |
| | | ACCAGCACCGCCTACATGGAACTGA |
| | | GCAGCCTGCGGAGCGAGGACACAGC |
| | | CGTGTATTACTGTGCCCGCGAGGCC |
| | | GTGGCCGCCGACTGGCTGGATCCTT |
| | | GGGGACAGGGCACCCTGGTGACAGT |
| | | GTCCAGC |
| | 5 | QVQLVQSGAEVKKPGSSVKVSCKDS |
| | | GGTFSSYAISWVRQAPGQGLEWMGG |
| | | IIPIFGTTNYAQQFQGRVTITADES |
| | | TSTAYMELSSLRSEDTAVYYCAREA |
| | | VAADWLDPWGQGTLVTVSS |
| CD8α | 57 | TTCGTGCCAGTGTTTCTACCTGCCA |
| | | AGCCGACCACCACGCCTGCCCCTAG |
| | | ACCTCCTACACCCGCCCCTACAATC |
| | | GCCAGCCAGCCTCTGTCTCTGAGGC |
| | | CCGAGGCTTGTAGACCTGCTGCTGG |
| | | CGGAGCCGTGCACACCAGAGGACTG |
| | | GATTTCGCCTGCGACATCTACATCT |
| | | GGGCCCCTCTGGCCGGCACATGTGG |
| | | CGTGCTGCTGCTCAGCCTGGTCATC |
| | | ACCCTGTACTGTAACCACCGGAAC |
| | 31 | FVPVFLPAKPTTTPAPRPPTPAPTI |
| | | ASQPLSLRPEACRPAAGGAVHTRGL |
| | | DFACDIYIWAPLAGTCGVLLLSLVI |
| | | TLYCNHRN |
| CD28 | 58 | AGAAGCAAGCGGAGCAGACTGCTGC |
| | | ACAGCGACTACATGAACATGACCCC |
| | | TAGACGGCCCGGACCTACCAGAAAG |
| | | CACTACCAGCCTTACGCTCCTCCTC |
| | | GGGACTTTGCCGCCTATCGGAGC |
| | 32 | RSKRSRLLHSDYMNMTPRRPGPTRK |
| | | HYQPYAPPRDFAAYRS |
| CD3ζ | 59 | AGAGTGAAGTTCAGCAGATCAGCCG |
| | | ATGCTCCTGCCTACCAGCAGGGCCA |
| | | GAATCAGCTGTACAACGAGCTGAAC |
| | | CTGGGGAGAAGAGAAGAGTACGACG |
| | | TGCTGGATAAGCGGAGAGGCAGAGA |
| | | TCCTGAGATGGGCGGCAAGCCCAGA |
| | | CGGAAGAATCCTCAGGAGGGCCTGT |
| | | ATAATGAGCTGCAGAAAGACAAGAT |
| | | GGCCGAGGCCTACAGCGAGATCGGC |
| | | ATGAAAGGCGAGAGAAGAAGAGGCA |
| | | AGGGCCACGATGGACTGTACCAGGG |
| | | ACTGAGCACAGCCACCAAGGATACC |
| | | TACGATGCCCTGCACATGCAGGCCC |
| | | TTCCACCTAGA |
| | 33 | RVKFSRSADAPAYQQGQNQLYNELN |
| | | LGRREEYDVLDKRRGRDPEMGGKPR |
| | | RKNPQEGLYNELQKDKMAEAYSEIG |
| | | MKGERRRGKGHDGLYQGLSTATKDT |
| | | YDALHMQALPPR |
| Cleavage | 60 | AGGGCCAAGAGATCTGGATCTGGCG |
| sequence | | CCCCTGTGAAGCAGACCCTGAATTT |
| | | CGACCTGCTGAAGCTGGCCGGCGAC |
| | | GTGGAATCTAATCCTGGACCT |
| | 37 | RAKRSGSGAPVKQTLNFDLLKLAGD |
| | | VESNPGP |
| GM-CSF SS | 61 | ATGCTTCTCCTGGTGACAAGCCTTC |
| | | TGCTCTGTGAGTTACCACACCCAGC |
| | | ATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTC |
| | | ACAGCAGCCCTGTTACACTGGGACA |
| | | GCCTGCCAGCATCTCCTGTAGAAGC |
| | | TCCCAGAGCCTGGTGTCCAGAGATG |
| | | GCAATACCTACCTGAGCTGGCTGCA |
| | | GCAGAGGCCTGGACAACCTCCTAGG |
| | | CTGCTGATTTACAAGATCAGCAACC |
| | | GGTTCAGCGGCGTGCCCAATAGATT |
| | | TTCTGGAAGCGGAGCCGGCACAGAC |
| | | TTTACCCTGAAGATTTCTAGAGTGA |
| | | AGGCCGAGGACGTGGGCGTGTACTA |
| | | CTGTATGCAGGCCACACAGTTCCCT |
| | | CTGACCTTTGGCCAGGGCACCAGAC |
| | | TGGAAATCAAA |
| | 14 | DIVMTQTPHSSPVTLGQPASISCRS |
| | | SQSLVSRDGNTYLSWLQQRPGQPPR |
| | | LLIYKISNRFSGVPNRFSGSGAGTD |
| | | FTLKISRVKAEDVGVYYCMQATQFP |
| | | LTFGQGTRLEIK |
| Linker | 68 | GGAGGAGGCGGGAGTGGTGGCGGAG |
| | | GTTCCGGCGGCGGAGGATCAGGCGG |
| | | AGGTGGAAGT |
| | 41 | GGGGSGGGGSGGGGSGGGGS |
| 2.1.2 HC | 64 | GAAGTCCAGCTCGTTCAGTCCGGAG |
| | | CCGAGGTGAAGAAGCCTGGCGAGTC |
| | | TCTGAAGATCAGCTGCAAAGGCAGC |
| | | GGCTACAGCTTCACCAGCTATTGGA |
| | | TCGGCTGGGTCCGACAGATGCCTGG |
| | | CAAAGGACTGGAGTGGATGGGCATC |
| | | ATCTACCCCGGCGACAGCGATACCA |
| | | GATACAGCCCTAGCTTTCAGGGCCA |
| | | AGTGACCATCAGCGCCGACAAGAGC |
| | | ATCAGCACAGCCTACCTGCAGTGGT |
| | | CTAGCCTGAAGGCCAGCGACACCGC |
| | | CATGTACTATTGTGCCAGACAGGGC |
| | | GACTTTTGGAGCGGCTATGGTGGCA |
| | | TGGATGTGTGGGGCCAGGGCACAAC |
| | | AGTGACCGTGTCTAGC |
| | 13 | EVQLVQSGAEVKKPGESLKISCKGS |
| | | GYSFTSYWIGWVRQMPGKGLEWMGI |
| | | IYPGDSDTRYSPSFQGQVTISADKS |
| | | ISTAYLQWSSLKASDTAMYYCARQG |
| | | DFWSGYGGMDVWGQGTTVTVSS |
| CD8α | 65 | TTCGTTCCGGTTTTTCTGCCGGCAA |
| | | AGCCTACAACTACCCCCGCACCCCG |
| | | GCCCCCAACTCCCGCTCCAACGATC |
| | | GCATCACAACCACTTTCACTCCGAC |
| | | CAGAGGCTTGCAGACCGGCTGCGGG |
| | | AGGCGCGGTACACACGCGGGGGCTC |
| | | GATTTTGCTTGCGATATTTACATTT |
| | | GGGCTCCTCTTGCCGGTACATGCGG |
| | | TGTCTTGCTCCTGTCCCTCGTCATT |
| | | ACTCTCTATTGCAACCATAGGAAC |
| | 31 | FVPVFLPAKPTTTPAPRPPTPAPTI |
| | | ASQPLSLRPEACRPAAGGAVHTRGL |
| | | DFACDIYIWAPLAGTCGVLLLSLVI |
| | | TLYCNHRN |
| CD28 | 69 | AGGAGTAAGAGGAGCAGGCTCCTGC |
| | | ATAGTGATTATATGAATATGACTCC |
| | | CCGCCGCCCCGGGCCCACCCGCAAG |
| | | CATTATCAGCCCTATGCCCCACCAC |
| | | GCGACTTCGCAGCCTACCGCTCC |
| | 32 | RSKRSRLLHSDYMNMTPRRPGPTRK |
| | | HYQPYAPPRDFAAYRS |
| CD3ζ | 67 | AGGGTGAAATTCTCTAGAAGCGCCG |
| | | ACGCACCCGCATATCAGCAAGGACA |
| | | AAACCAGCTCTATAACGAACTCAAC |
| | | CTCGGCAGACGCGAGGAATATGATG |
| | | TGCTGGACAAGAGGCGGGGACGCGA |
| | | TCCAGAAATGGGAGGAAAGCCTCGG |
| | | AGAAAGAACCCACAAGAGGGACTTT |
| | | ACAACGAACTCCAAAAGGATAAGAT |
| | | GGCAGAAGCCTATTCCGAGATTGGA |
| | | ATGAAGGGCGAACGTCGGAGAGGAA |
| | | AGGGACACGACGGCCTTTATCAGGG |
| | | CCTGTCCACCGCCACAAAAGATACG |
| | | TATGACGCTCTCCACATGCAAGCGT |
| | | TGCCCCCCCGC |
| | 33 | RVKFSRSADAPAYQQGQNQLYNELN |
| | | LGRREEYDVLDKRRGRDPEMGGKPR |
| | | RKNPQEGLYNELQKDKMAEAYSEIG |
| | | MKGERRRGKGHDGLYQGLSTATKDT |
| | | YDALHMQALPPR |

TABLE 4
Hu1928-Hu2028 std (standard)

| | SEQ ID | |
|---|---|---|
| Description | NO: | Sequence |
| CD8α SS | 53 | ATGGCCCTGCCTGTGACAGCTCTGCTGCTGCCCCTGGC |
| | | CCTGCTGCTGCATGCCGCCAGACCT |
| | 30 | MALPVTALLLPLALLLHAARP |
| CD19 scFv: | | |
| 47G4 LC | 54 | GAGATCGTGCTGACCCAGTCTCCCGGTACCCTGTCTCT |
| | | CAGCCCAGGAGAGAGAGCCACCCTGAGCTGCAGAGCC |
| | | AGCCAGAGCGTGTCCAGCAGCTACCTGGCCTGGTATCA |
| | | GCAGAAGCCCGGACAGGCCCCCAGACTGCTGATCTAC |
| | | GGCGCCAGCTCTAGAGCCACCGGCATCCCCGACAGATT |
| | | CAGCGGCAGCGGCAGTGGTACCGACTTCACCCTGACC |
| | | ATCAGCAGACTGGAACCCGAGGACTTCGCCGTGTATTA |
| | | CTGCCAGCAGTACGGCAGCAGCCGGTTCACCTTCGGCC |
| | | CTGGCACCAAGGTGGACATCAAG |
| | 6 | EIVLTQSPGTLSLSPGERATLSCRASQSVSSSYLAWYQQK |
| | | PGQAPRLLIYGASSRATGIPDRFSGSGSGTDFTLTISRLEPE |
| | | DFAVYYCQQYGSSRFTFGPGTKVDIK |
| Linker | 55 | GGCAGCACCTCCGGCAGCGGCAAGCCTGGCTCTGGCG |
| | | AGGGCTCTACCAAGGGC |
| | 29 | GSTSGSGKPGSGEGSTKG |
| 47G4 HC | 56 | CAGGTGCAGCTGGTGCAGTCTGGCGCCGAAGTCAAGA |
| | | AACCCGGCTCTAGCGTGAAGGTGTCCTGCAAGGACAG |
| | | CGGCGGCACCTTCAGCAGCTACGCCATCAGCTGGGTGC |
| | | GCCAGGCCCCAGGACAGGGGCTGGAATGGATGGGCGG |
| | | CATCATCCCCATCTTCGGCACCACCAACTACGCCCAGC |
| | | AGTTCCAGGGCAGAGTGACCATCACCGCCGACGAGAG |
| | | CACCAGCACCGCCTACATGGAACTGAGCAGCCTGCGG |
| | | AGCGAGGACACAGCCGTGTATTACTGTGCCCGCGAGG |
| | | CCGTGGCCGCCGACTGGCTGGATCCTTGGGGACAGGG |
| | | CACCCTGGTGACAGTGTCCAGC |
| | 5 | QVQLVQSGAEVKKPGSSVKVSCKDSGGTFSSYAISWVRQ |
| | | APGQGLEWMGGIIPIFGTTNYAQQFQGRVTITADESTSTA |
| | | YMELSSLRSEDTAVYYCAREAVAADWLDPWGQGTLVT |
| | | VSS |
| CD8α | 57 | TTCGTGCCAGTGTTTCTACCTGCCAAGCCGACCACCAC |
| | | GCCTGCCCCTAGACCTCCTACACCCGCCCCTACAATCG |
| | | CCAGCCAGCCTCTGTCTCTGAGGCCCGAGGCTTGTAGA |
| | | CCTGCTGCTGGCGGAGCCGTGCACACCAGAGGACTGG |
| | | ATTTCGCCTGCGACATCTACATCTGGGCCCCTCTGGCC |
| | | GGCACATGTGGCGTGCTGCTGCTCAGCCTGGTCATCAC |
| | | CCTGTACTGTAACCACCGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| CD28 | 58 | AGAAGCAAGCGGAGCAGACTGCTGCACAGCGACTACA |
| | | TGAACATGACCCCTAGACGGCCCGGACCTACCAGAAA |
| | | GCACTACCAGCCTTACGCTCCTCCTCGGGACTTTGCCG |
| | | CCTATCGGAGC |
| | 32 | RSKRSRLLHSDYMNMTPRRPGPTRKHYQPYAPPRDFAAY |
| | | RS |
| CD3ζ | 59 | AGAGTGAAGTTCAGCAGATCAGCCGATGCTCCTGCCTA |
| | | CCAGCAGGGCCAGAATCAGCTGTACAACGAGCTGAAC |
| | | CTGGGGAGAAGAGAAGAGTACGACGTGCTGGATAAGC |
| | | GGAGAGGCAGAGATCCTGAGATGGGCGGCAAGCCCAG |
| | | ACGGAAGAATCCTCAGGAGGGCCTGTATAATGAGCTG |
| | | CAGAAAGACAAGATGGCCGAGGCCTACAGCGAGATCG |
| | | GCATGAAAGGCGAGAGAAGAAGAGGCAAGGGCCACG |
| | | ATGGACTGTACCAGGGACTGAGCACAGCCACCAAGGA |
| | | TACCTACGATGCCCTGCACATGCAGGCCCTTCCACCTA |
| | | GA |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |
| Cleavage | 60 | AGGGCCAAGAGATCTGGATCTGGCGCCCCTGTGAAGC |
| sequence | | AGACCCTGAATTTCGACCTGCTGAAGCTGGCCGGCGAC |
| | | GTGGAATCTAATCCTGGACCT |
| | 37 | RAKRSGSGAPVKQTLNFDLLKLAGDVESNPGP |
| GM-CSF SS | 61 | ATGCTTCTCCTGGTGACAAGCCTTCTGCTCTGTGAGTTA |
| | | CCACACCCAGCATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTCACAGCAGCCCTGT |
| | | TACACTGGGACAGCCTGCCAGCATCTCCTGTAGAAGCT |
| | | CCCAGAGCCTGGTGTCCAGAGATGGCAATACCTACCTG |
| | | AGCTGGCTGCAGCAGAGGCCTGGACAACCTCCTAGGC |
| | | TGCTGATTTACAAGATCAGCAACCGGTTCAGCGGCGTG |
| | | CCCAATAGATTTTCTGGAAGCGGAGCCGGCACAGACTT |
| | | TACCCTGAAGATTTCTAGAGTGAAGGCCGAGGACGTG |
| | | GGCGTGTACTACTGTATGCAGGCCACACAGTTCCCTCT |
| | | GACCTTTGGCCAGGGCACCAGACTGGAAATCAAA |
| | 14 | DIVMTQTPHSSPVTLGQPASISCRSSQSLVSRDGNTYLSW |
| | | LQQRPGQPPRLLIYKISNRFSGVPNRFSGSGAGTDFTLKIS |
| | | RVKAEDVGVYYCMQATQFPLTFGQGTRLEIK |
| Linker | 63 | GGTGGCGGAGGTTCCGGCGGCGGAGGATCAGGCGGAG |
| | | GTGGAAGT |
| | 10 | GGGGSGGGGSGGGGS |
| 2.1.2 HC | 64 | GAAGTCCAGCTCGTTCAGTCCGGAGCCGAGGTGAAGA |
| | | AGCCTGGCGAGTCTCTGAAGATCAGCTGCAAAGGCAG |
| | | CGGCTACAGCTTCACCAGCTATTGGATCGGCTGGGTCC |
| | | GACAGATGCCTGGCAAAGGACTGGAGTGGATGGGCAT |
| | | CATCTACCCCGGCGACAGCGATACCAGATACAGCCCTA |
| | | GCTTTCAGGGCCAAGTGACCATCAGCGCCGACAAGAG |
| | | CATCAGCACAGCCTACCTGCAGTGGTCTAGCCTGAAGG |
| | | CCAGCGACACCGCCATGTACTATTGTGCCAGACAGGGC |
| | | GACTTTTGGAGCGGCTATGGTGGCATGGATGTGTGGGG |
| | | CCAGGGCACAACAGTGACCGTGTCTAGC |
| | 13 | EVQLVQSGAEVKKPGESLKISCKGSGYSFTSYWIGWVRQ |
| | | MPGKGLEWMGIIYPGDSDTRYSPSFQGQVTISADKSISTA |
| | | YLQWSSLKASDTAMYYCARQGDFWSGYGGMDVWGQG |
| | | TTVTVSS |
| CD8α | 65 | TTCGTTCCGGTTTTTCTGCCGGCAAAGCCTACAACTAC |
| | | CCCCGCACCCCGGCCCCCAACTCCCGCTCCAACGATCG |
| | | CATCACAACCACTTTCACTCCGACCAGAGGCTTGCAGA |
| | | CCGGCTGCGGGAGGCGCGGTACACACGCGGGGGCTCG |
| | | ATTTTGCTTGCGATATTTACATTTGGGCTCCTCTTGCCG |
| | | GTACATGCGGTGTCTTGCTCCTGTCCCTCGTCATTACTC |
| | | TCTATTGCAACCATAGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| CD28 | 69 | AGGAGTAAGAGGAGCAGGCTCCTGCATAGTGATTATA |
| | | TGAATATGACTCCCCGCCGCCCCGGGCCCACCCGCAAG |
| | | CATTATCAGCCCTATGCCCCACCACGCGACTTCGCAGC |
| | | CTACCGCTCC |
| | 32 | RSKRSRLLHSDYMNMTPRRPGPTRKHYQPYAPPRDFAAY |
| | | RS |
| CD3ζ | 67 | AGGGTGAAATTCTCTAGAAGCGCCGACGCACCCGCAT |
| | | ATCAGCAAGGACAAAACCAGCTCTATAACGAACTCAA |
| | | CCTCGGCAGACGCGAGGAATATGATGTGCTGGACAAG |
| | | AGGCGGGGACGCGATCCAGAAATGGGAGGAAAGCCTC |
| | | GGAGAAAGAACCCACAAGAGGGACTTTACAACGAACT |
| | | CCAAAAGGATAAGATGGCAGAAGCCTATTCCGAGATT |
| | | GGAATGAAGGGCGAACGTCGGAGAGGAAAGGGACAC |
| | | GACGGCCTTTATCAGGGCCTGTCCACCGCCACAAAAGA |
| | | TACGTATGACGCTCTCCACATGCAAGCGTTGCCCCCCC |
| | | GC |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |

TABLE 5
10-5-2020 Hu19-CD828Z

| | SEQ ID | |
|---|---|---|
| Description | NO: | Sequence |
| CD8α SS | 53 | ATGGCCCTGCCTGTGACAGCTCTGCTGCTGCCCCTGGC |
| | | CCTGCTGCTGCATGCCGCCAGACCT |
| | 30 | MALPVTALLLPLALLLHAARP |
| CD19 scFv: | | |
| 47G4 LC | 54 | GAGATCGTGCTGACCCAGTCTCCCGGTACCCTGTCTCT |
| | | CAGCCCAGGAGAGAGAGCCACCCTGAGCTGCAGAGCC |
| | | AGCCAGAGCGTGTCCAGCAGCTACCTGGCCTGGTATCA |
| | | GCAGAAGCCCGGACAGGCCCCCAGACTGCTGATCTAC |
| | | GGCGCCAGCTCTAGAGCCACCGGCATCCCCGACAGATT |
| | | CAGCGGCAGCGGCAGTGGTACCGACTTCACCCTGACC |
| | | ATCAGCAGACTGGAACCCGAGGACTTCGCCGTGTATTA |
| | | CTGCCAGCAGTACGGCAGCAGCCGGTTCACCTTCGGCC |
| | | CTGGCACCAAGGTGGACATCAAG |
| | 6 | EIVLTQSPGTLSLSPGERATLSCRASQSVSSSYLAWYQQK |
| | | PGQAPRLLIYGASSRATGIPDRFSGSGSGTDFTLTISRLEPE |
| | | DFAVYYCQQYGSSRFTFGPGTKVDIK |
| Linker | 55 | GGCAGCACCTCCGGCAGCGGCAAGCCTGGCTCTGGCG |
| | | AGGGCTCTACCAAGGGC |
| | 29 | GSTSGSGKPGSGEGSTKG |
| 47G4 HC | 56 | CAGGTGCAGCTGGTGCAGTCTGGCGCCGAAGTCAAGA |
| | | AACCCGGCTCTAGCGTGAAGGTGTCCTGCAAGGACAG |
| | | CGGCGGCACCTTCAGCAGCTACGCCATCAGCTGGGTGC |
| | | GCCAGGCCCCAGGACAGGGGCTGGAATGGATGGGCGG |
| | | CATCATCCCCATCTTCGGCACCACCAACTACGCCCAGC |
| | | AGTTCCAGGGCAGAGTGACCATCACCGCCGACGAGAG |
| | | CACCAGCACCGCCTACATGGAACTGAGCAGCCTGCGG |
| | | AGCGAGGACACAGCCGTGTATTACTGTGCCCGCGAGG |
| | | CCGTGGCCGCCGACTGGCTGGATCCTTGGGGACAGGG |
| | | CACCCTGGTGACAGTGTCCAGC |
| | 5 | QVQLVQSGAEVKKPGSSVKVSCKDSGGTFSSYAISWVRQ |
| | | APGQGLEWMGGIIPIFGTTNYAQQFQGRVTITADESTSTA |
| | | YMELSSLRSEDTAVYYCAREAVAADWLDPWGQGTLVT |
| | | VSS |
| CD8α | 57 | TTCGTGCCAGTGTTTCTACCTGCCAAGCCGACCACCAC |
| | | GCCTGCCCCTAGACCTCCTACACCCGCCCCTACAATCG |
| | | CCAGCCAGCCTCTGTCTCTGAGGCCCGAGGCTTGTAGA |
| | | CCTGCTGCTGGCGGAGCCGTGCACACCAGAGGACTGG |
| | | ATTTCGCCTGCGACATCTACATCTGGGCCCCTCTGGCC |
| | | GGCACATGTGGCGTGCTGCTGCTCAGCCTGGTCATCAC |
| | | CCTGTACTGTAACCACCGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| CD28 | 58 | AGAAGCAAGCGGAGCAGACTGCTGCACAGCGACTACA |
| | | TGAACATGACCCCTAGACGGCCCGGACCTACCAGAAA |
| | | GCACTACCAGCCTTACGCTCCTCCTCGGGACTTTGCCG |
| | | CCTATCGGAGC |
| | 32 | RSKRSRLLHSDYMNMTPRRPGPTRKHYQPYAPPRDFAAY |
| | | RS |
| CD3ζ | 59 | AGAGTGAAGTTCAGCAGATCAGCCGATGCTCCTGCCTA |
| | | CCAGCAGGGCCAGAATCAGCTGTACAACGAGCTGAAC |
| | | CTGGGGAGAAGAGAAGAGTACGACGTGCTGGATAAGC |
| | | GGAGAGGCAGAGATCCTGAGATGGGCGGCAAGCCCAG |
| | | ACGGAAGAATCCTCAGGAGGGCCTGTATAATGAGCTG |
| | | CAGAAAGACAAGATGGCCGAGGCCTACAGCGAGATCG |
| | | GCATGAAAGGCGAGAGAAGAAGAGGCAAGGGCCACG |
| | | ATGGACTGTACCAGGGACTGAGCACAGCCACCAAGGA |
| | | TACCTACGATGCCCTGCACATGCAGGCCCTTCCACCTA |
| | | GA |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |

TABLE 6
9-15-2020 Hu20-CD8BBZ std

| | SEQ ID | |
|---|---|---|
| Description | NO: | Sequence |
| GM-CSF SS | 61 | ATGCTTCTCCTGGTGACAAGCCTTCTGCTCTGTGAGTTA |
| | | CCACACCCAGCATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTCACAGCAGCCCTGT |
| | | TACACTGGGACAGCCTGCCAGCATCTCCTGTAGAAGCT |
| | | CCCAGAGCCTGGTGTCCAGAGATGGCAATACCTACCTG |
| | | AGCTGGCTGCAGCAGAGGCCTGGACAACCTCCTAGGC |
| | | TGCTGATTTACAAGATCAGCAACCGGTTCAGCGGCGTG |
| | | CCCAATAGATTTTCTGGAAGCGGAGCCGGCACAGACTT |
| | | TACCCTGAAGATTTCTAGAGTGAAGGCCGAGGACGTG |
| | | GGCGTGTACTACTGTATGCAGGCCACACAGTTCCCTCT |
| | | GACCTTTGGCCAGGGCACCAGACTGGAAATCAAA |
| | 14 | DIVMTQTPHSSPVTLGQPASISCRSSQSLVSRDGNTYLSW |
| | | LQQRPGQPPRLLIYKISNRFSGVPNRFSGSGAGTDFTLKIS |
| | | RVKAEDVGVYYCMQATQFPLTFGQGTRLEIK |
| Linker | 63 | GGTGGCGGAGGTTCCGGCGGCGGAGGATCAGGCGGAG |
| | | GTGGAAGT |
| | 10 | GGGGSGGGGSGGGGS |
| 2.1.2 HC | 64 | GAAGTCCAGCTCGTTCAGTCCGGAGCCGAGGTGAAGA |
| | | AGCCTGGCGAGTCTCTGAAGATCAGCTGCAAAGGCAG |
| | | CGGCTACAGCTTCACCAGCTATTGGATCGGCTGGGTCC |
| | | GACAGATGCCTGGCAAAGGACTGGAGTGGATGGGCAT |
| | | CATCTACCCCGGCGACAGCGATACCAGATACAGCCCTA |
| | | GCTTTCAGGGCCAAGTGACCATCAGCGCCGACAAGAG |
| | | CATCAGCACAGCCTACCTGCAGTGGTCTAGCCTGAAGG |
| | | CCAGCGACACCGCCATGTACTATTGTGCCAGACAGGGC |
| | | GACTTTTGGAGCGGCTATGGTGGCATGGATGTGTGGGG |
| | | CCAGGGCACAACAGTGACCGTGTCTAGC |
| | 13 | EVQLVQSGAEVKKPGESLKISCKGSGYSFTSYWIGWVRQ |
| | | MPGKGLEWMGIIYPGDSDTRYSPSFQGQVTISADKSISTA |
| | | YLQWSSLKASDTAMYYCARQGDFWSGYGGMDVWGQG |
| | | TTVTVSS |
| CD8α | 65 | TTCGTTCCGGTTTTTCTGCCGGCAAAGCCTACAACTAC |
| | | CCCCGCACCCCGGCCCCCAACTCCCGCTCCAACGATCG |
| | | CATCACAACCACTTTCACTCCGACCAGAGGCTTGCAGA |
| | | CCGGCTGCGGGAGGCGCGGTACACACGCGGGGGCTCG |
| | | ATTTTGCTTGCGATATTTACATTTGGGCTCCTCTTGCCG |
| | | GTACATGCGGTGTCTTGCTCCTGTCCCTCGTCATTACTC |
| | | TCTATTGCAACCATAGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| 4-1BB | 66 | AAGCGAGGCCGGAAGAAGCTGCTGTACATCTTCAAGC |
| | | AGCCTTTCATGCGGCCCGTGCAGACCACACAAGAGGA |
| | | AGATGGCTGTAGCTGCAGATTCCCCGAGGAAGAAGAA |
| | | GGCGGCTGCGAGCTG |
| | 34 | KRGRKKLLYIFKQPFMRPVQTTQEEDGCSCRFPEEEEGGC |
| | | EL |
| CD3ζ | 67 | AGGGTGAAATTCTCTAGAAGCGCCGACGCACCCGCAT |
| | | ATCAGCAAGGACAAAACCAGCTCTATAACGAACTCAA |
| | | CCTCGGCAGACGCGAGGAATATGATGTGCTGGACAAG |
| | | AGGCGGGGACGCGATCCAGAAATGGGAGGAAAGCCTC |
| | | GGAGAAAGAACCCACAAGAGGGACTTTACAACGAACT |
| | | CCAAAAGGATAAGATGGCAGAAGCCTATTCCGAGATT |
| | | GGAATGAAGGGCGAACGTCGGAGAGGAAAGGGACAC |
| | | GACGGCCTTTATCAGGGCCTGTCCACCGCCACAAAAGA |
| | | TACGTATGACGCTCTCCACATGCAAGCGTTGCCCCCCC |
| | | GC |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |

TABLE 7
Hu20-CD828Z std

| | SEQ ID | |
|---|---|---|
| Description | NO: | Sequence |
| GM-CSF SS | 61 | ATGCTTCTCCTGGTGACAAGCCTTCTGCTCTGTGAGTTA |
| | | CCACACCCAGCATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTCACAGCAGCCCTGT |
| | | TACACTGGGACAGCCTGCCAGCATCTCCTGTAGAAGCT |
| | | CCCAGAGCCTGGTGTCCAGAGATGGCAATACCTACCTG |
| | | AGCTGGCTGCAGCAGAGGCCTGGACAACCTCCTAGGC |
| | | TGCTGATTTACAAGATCAGCAACCGGTTCAGCGGCGTG |
| | | CCCAATAGATTTTCTGGAAGCGGAGCCGGCACAGACTT |
| | | TACCCTGAAGATTTCTAGAGTGAAGGCCGAGGACGTG |
| | | GGCGTGTACTACTGTATGCAGGCCACACAGTTCCCTCT |
| | | GACCTTTGGCCAGGGCACCAGACTGGAAATCAAA |
| | 14 | DIVMTQTPHSSPVTLGQPASISCRSSQSLVSRDGNTYLSW |
| | | LQQRPGQPPRLLIYKISNRFSGVPNRFSGSGAGTDFTLKIS |
| | | RVKAEDVGVYYCMQATQFPLTFGQGTRLEIK |
| Linker | 63 | GGTGGCGGAGGTTCCGGCGGCGGAGGATCAGGCGGAG |
| | | GTGGAAGT |
| | 10 | GGGGSGGGGSGGGGS |
| 2.1.2 HC | 64 | GAAGTCCAGCTCGTTCAGTCCGGAGCCGAGGTGAAGA |
| | | AGCCTGGCGAGTCTCTGAAGATCAGCTGCAAAGGCAG |
| | | CGGCTACAGCTTCACCAGCTATTGGATCGGCTGGGTCC |
| | | GACAGATGCCTGGCAAAGGACTGGAGTGGATGGGCAT |
| | | CATCTACCCCGGCGACAGCGATACCAGATACAGCCCTA |
| | | GCTTTCAGGGCCAAGTGACCATCAGCGCCGACAAGAG |
| | | CATCAGCACAGCCTACCTGCAGTGGTCTAGCCTGAAGG |
| | | CCAGCGACACCGCCATGTACTATTGTGCCAGACAGGGC |
| | | GACTTTTGGAGCGGCTATGGTGGCATGGATGTGTGGGG |
| | | CCAGGGCACAACAGTGACCGTGTCTAGC |
| | 13 | EVQLVQSGAEVKKPGESLKISCKGSGYSFTSYWIGWVRQ |
| | | MPGKGLEWMGIIYPGDSDTRYSPSFQGQVTISADKSISTA |
| | | YLQWSSLKASDTAMYYCARQGDFWSGYGGMDVWGQG |
| | | TTVTVSS |
| CD8α | 65 | TTCGTTCCGGTTTTTCTGCCGGCAAAGCCTACAACTAC |
| | | CCCCGCACCCCGGCCCCCAACTCCCGCTCCAACGATCG |
| | | CATCACAACCACTTTCACTCCGACCAGAGGCTTGCAGA |
| | | CCGGCTGCGGGAGGCGCGGTACACACGCGGGGGCTCG |
| | | ATTTTGCTTGCGATATTTACATTTGGGCTCCTCTTGCCG |
| | | GTACATGCGGTGTCTTGCTCCTGTCCCTCGTCATTACTC |
| | | TCTATTGCAACCATAGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| CD28 | 69 | AGGAGTAAGAGGAGCAGGCTCCTGCATAGTGATTATA |
| | | TGAATATGACTCCCCGCCGCCCCGGGCCCACCCGCAAG |
| | | CATTATCAGCCCTATGCCCCACCACGCGACTTCGCAGC |
| | | CTACCGCTCC |
| | 32 | RSKRSRLLHSDYMNMTPRRPGPTRKHYQPYAPPRDFAAY |
| | | RS |
| CD3ζ | 67 | AGGGTGAAATTCTCTAGAAGCGCCGACGCACCCGCAT |
| | | ATCAGCAAGGACAAAACCAGCTCTATAACGAACTCAA |
| | | CCTCGGCAGACGCGAGGAATATGATGTGCTGGACAAG |
| | | AGGCGGGGACGCGATCCAGAAATGGGAGGAAAGCCTC |
| | | GGAGAAAGAACCCACAAGAGGGACTTTACAACGAACT |
| | | CCAAAAGGATAAGATGGCAGAAGCCTATTCCGAGATT |
| | | GGAATGAAGGGCGAACGTCGGAGAGGAAAGGGACAC |
| | | GACGGCCTTTATCAGGGCCTGTCCACCGCCACAAAAGA |
| | | TACGTATGACGCTCTCCACATGCAAGCGTTGCCCCCCC |
| | | GC |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |

TABLE 8
Hu20-CD8BBZ long

| | SEQ ID | |
|---|---|---|
| Description | NO: | Sequence |
| GM-CSF SS | 61 | ATGCTTCTCCTGGTGACAAGCCTTCTGCTCTGTGAGTTA |
| | | CCACACCCAGCATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTCACAGCAGCCCTGT |
| | | TACACTGGGACAGCCTGCCAGCATCTCCTGTAGAAGCT |
| | | CCCAGAGCCTGGTGTCCAGAGATGGCAATACCTACCTG |
| | | AGCTGGCTGCAGCAGAGGCCTGGACAACCTCCTAGGC |
| | | TGCTGATTTACAAGATCAGCAACCGGTTCAGCGGCGTG |
| | | CCCAATAGATTTTCTGGAAGCGGAGCCGGCACAGACTT |
| | | TACCCTGAAGATTTCTAGAGTGAAGGCCGAGGACGTG |
| | | GGCGTGTACTACTGTATGCAGGCCACACAGTTCCCTCT |
| | | GACCTTTGGCCAGGGCACCAGACTGGAAATCAAA |
| | 14 | DIVMTQTPHSSPVTLGQPASISCRSSQSLVSRDGNTYLSW |
| | | LQQRPGQPPRLLIYKISNRFSGVPNRFSGSGAGTDFTLKIS |
| | | RVKAEDVGVYYCMQATQFPLTFGQGTRLEIK |
| Linker | 68 | GGAGGAGGCGGGAGTGGTGGCGGAGGTTCCGGCGGCG |
| | | GAGGATCAGGCGGAGGTGGAAGT |
| | 41 | GGGGSGGGGSGGGGSGGGGS |
| 2.1.2 HC | 64 | GAAGTCCAGCTCGTTCAGTCCGGAGCCGAGGTGAAGA |
| | | AGCCTGGCGAGTCTCTGAAGATCAGCTGCAAAGGCAG |
| | | CGGCTACAGCTTCACCAGCTATTGGATCGGCTGGGTCC |
| | | GACAGATGCCTGGCAAAGGACTGGAGTGGATGGGCAT |
| | | CATCTACCCCGGCGACAGCGATACCAGATACAGCCCTA |
| | | GCTTTCAGGGCCAAGTGACCATCAGCGCCGACAAGAG |
| | | CATCAGCACAGCCTACCTGCAGTGGTCTAGCCTGAAGG |
| | | CCAGCGACACCGCCATGTACTATTGTGCCAGACAGGGC |
| | | GACTTTTGGAGCGGCTATGGTGGCATGGATGTGTGGGG |
| | | CCAGGGCACAACAGTGACCGTGTCTAGC |
| | 13 | EVQLVQSGAEVKKPGESLKISCKGSGYSFTSYWIGWVRQ |
| | | MPGKGLEWMGIIYPGDSDTRYSPSFQGQVTISADKSISTA |
| | | YLQWSSLKASDTAMYYCARQGDFWSGYGGMDVWGQG |
| | | TTVTVSS |
| CD8α | 65 | TTCGTTCCGGTTTTTCTGCCGGCAAAGCCTACAACTAC |
| | | CCCCGCACCCCGGCCCCCAACTCCCGCTCCAACGATCG |
| | | CATCACAACCACTTTCACTCCGACCAGAGGCTTGCAGA |
| | | CCGGCTGCGGGAGGCGCGGTACACACGCGGGGGCTCG |
| | | ATTTTGCTTGCGATATTTACATTTGGGCTCCTCTTGCCG |
| | | GTACATGCGGTGTCTTGCTCCTGTCCCTCGTCATTACTC |
| | | TCTATTGCAACCATAGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| 4-1BB | 66 | AAGCGAGGCCGGAAGAAGCTGCTGTACATCTTCAAGC |
| | | AGCCTTTCATGCGGCCCGTGCAGACCACACAAGAGGA |
| | | AGATGGCTGTAGCTGCAGATTCCCCGAGGAAGAAGAA |
| | | GGCGGCTGCGAGCTG |
| | 34 | KRGRKKLLYIFKQPFMRPVQTTQEEDGCSCRFPEEEEGGC |
| | | EL |
| CD3ζ | 67 | AGGGTGAAATTCTCTAGAAGCGCCGACGCACCCGCAT |
| | | ATCAGCAAGGACAAAACCAGCTCTATAACGAACTCAA |
| | | CCTCGGCAGACGCGAGGAATATGATGTGCTGGACAAG |
| | | AGGCGGGGACGCGATCCAGAAATGGGAGGAAAGCCTC |
| | | GGAGAAAGAACCCACAAGAGGGACTTTACAACGAACT |
| | | CCAAAAGGATAAGATGGCAGAAGCCTATTCCGAGATT |
| | | GGAATGAAGGGCGAACGTCGGAGAGGAAAGGGACAC |
| | | GACGGCCTTTATCAGGGCCTGTCCACCGCCACAAAAGA |
| | | TACGTATGACGCTCTCCACATGCAAGCGTTGCCCCCCC |
| | | GC |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |

TABLE 9
Hu20-CD828Z long

| | SEQ ID | |
|---|---|---|
| Description | NO: | Sequence |
| GM-CSF SS | 61 | ATGCTTCTCCTGGTGACAAGCCTTCTGCTCTGTGAGTTA |
| | | CCACACCCAGCATTCCTCCTGATCCCA |
| | 80 | MLLLVTSLLLCELPHPAFLLIP |
| CD20 scFv: | | |
| 2.1.2 LC | 62 | GATATCGTGATGACACAGACACCTCACAGCAGCCCTGT |
| | | TACACTGGGACAGCCTGCCAGCATCTCCTGTAGAAGCT |
| | | CCCAGAGCCTGGTGTCCAGAGATGGCAATACCTACCTG |
| | | AGCTGGCTGCAGCAGAGGCCTGGACAACCTCCTAGGC |
| | | TGCTGATTTACAAGATCAGCAACCGGTTCAGCGGCGTG |
| | | CCCAATAGATTTTCTGGAAGCGGAGCCGGCACAGACTT |
| | | TACCCTGAAGATTTCTAGAGTGAAGGCCGAGGACGTG |
| | | GGCGTGTACTACTGTATGCAGGCCACACAGTTCCCTCT |
| | | GACCTTTGGCCAGGGCACCAGACTGGAAATCAAA |
| | 14 | DIVMTQTPHSSPVTLGQPASISCRSSQSLVSRDGNTYLSW |
| | | LQQRPGQPPRLLIYKISNRFSGVPNRFSGSGAGTDFTLKIS |
| | | RVKAEDVGVYYCMQATQFPLTFGQGTRLEIK |
| Linker | 68 | GGAGGAGGCGGGAGTGGTGGCGGAGGTTCCGGCGGCG |
| | | GAGGATCAGGCGGAGGTGGAAGT |
| | 41 | GGGGSGGGGSGGGGSGGGGS |
| 2.1.2 HC | 64 | GAAGTCCAGCTCGTTCAGTCCGGAGCCGAGGTGAAGA |
| | | AGCCTGGCGAGTCTCTGAAGATCAGCTGCAAAGGCAG |
| | | CGGCTACAGCTTCACCAGCTATTGGATCGGCTGGGTCC |
| | | GACAGATGCCTGGCAAAGGACTGGAGTGGATGGGCAT |
| | | CATCTACCCCGGCGACAGCGATACCAGATACAGCCCTA |
| | | GCTTTCAGGGCCAAGTGACCATCAGCGCCGACAAGAG |
| | | CATCAGCACAGCCTACCTGCAGTGGTCTAGCCTGAAGG |
| | | CCAGCGACACCGCCATGTACTATTGTGCCAGACAGGGC |
| | | GACTTTTGGAGCGGCTATGGTGGCATGGATGTGTGGGG |
| | | CCAGGGCACAACAGTGACCGTGTCTAGC |
| | 13 | EVQLVQSGAEVKKPGESLKISCKGSGYSFTSYWIGWVRQ |
| | | MPGKGLEWMGIIYPGDSDTRYSPSFQGQVTISADKSISTA |
| | | YLQWSSLKASDTAMYYCARQGDFWSGYGGMDVWGQG |
| | | TTVTVSS |
| CD8α | 65 | TTCGTTCCGGTTTTTCTGCCGGCAAAGCCTACAACTAC |
| | | CCCCGCACCCCGGCCCCCAACTCCCGCTCCAACGATCG |
| | | CATCACAACCACTTTCACTCCGACCAGAGGCTTGCAGA |
| | | CCGGCTGCGGGAGGCGCGGTACACACGCGGGGGCTCG |
| | | ATTTTGCTTGCGATATTTACATTTGGGCTCCTCTTGCCG |
| | | GTACATGCGGTGTCTTGCTCCTGTCCCTCGTCATTACTC |
| | | TCTATTGCAACCATAGGAAC |
| | 31 | FVPVFLPAKPTTTPAPRPPTPAPTIASQPLSLRPEACRPAAG |
| | | GAVHTRGLDFACDIYIWAPLAGTCGVLLLSLVITLYCNHR |
| | | N |
| CD28 | 69 | AGGAGTAAGAGGAGCAGGCTCCTGCATAGTGATTATA |
| | | TGAATATGACTCCCCGCCGCCCCGGGCCCACCCGCAAG |
| | | CATTATCAGCCCTATGCCCCACCACGCGACTTCGCAGC |
| | | CTACCGCTCC |
| | 32 | RSKRSRLLHSDYMNMTPRRPGPTRKHYQPYAPPRDFAAY |
| | | RS |
| CD3ζ | 67 | AGGGTGAAATTCTCTAGAAGCGCCGACGCACCCGCAT |
| | | ATCAGCAAGGACAAAACCAGCTCTATAACGAACTCAA |
| | | CCTCGGCAGACGCGAGGAATATGATGTGCTGGACAAG |
| | | AGGCGGGGACGCGATCCAGAAATGGGAGGAAAGCCTC |
| | | GGAGAAAGAACCCACAAGAGGGACTTTACAACGAACT |
| | | CCAAAAGGATAAGATGGCAGAAGCCTATTCCGAGATT |
| | | GGAATGAAGGGCGAACGTCGGAGAGGAAAGGGACAC |
| | | GACGGCCTTTATCAGGGCCTGTCCACCGCCACAAAAGA |
| | | TACGTATGACGCTCTCCACATGCAAGCGTTGCCCCCCC |
| | | GC |
| | 33 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |

T-Cell Culture

PBMC were thawed and washed in T cell medium that consisted of AIM V medium (Invitrogen, Carlsbad, CA, USA) plus 500 AB serum (Valley Biomedical, Winchester, VA, USA), 100 U/mL penicillin, and 100 μg/mL streptomycin. Prior to transductions, PBMC were suspended at a concentration of 1×10⁶ cells/mL in T cell medium plus 50 ng/mL of the anti-CD3 monoclonal antibody OKT3 (Ortho, Bridgewater, NJ, USA) and 300 IU/mL of interleukin-2 (IL-2). After transductions, T cells were maintained in T-cell medium plus TL,-2.

Gammaretroviral Transductions

To produce replication-incompetent gammaretroviruses, packaging cells were transfected with plasmids encoding CARs along with a plasmid encoding the RD 114 envelope protein as previously described, and gammaretroviral transduction of T cells was performed as previously described 2 days after initiation of T-cell cultures (Kochenderfer et al., Journal of Immunotherapy, 32:689-702 (2009), incorporated by reference herein).

CAR Detection on T Cells

An APC-labeled antibody that specifically binds to the linker component of the Hul9-CD828Z CAR was used to detect this CAR. A Pacific Blue-labeled antibody designated Kip-4 was used to detect anti-CD20 CARs. Kip-4 binds to the (G4S)3 linker in the Hu20 scFv. To do this staining, single-cell suspensions of T cells were prepared, and the cells were stained by standard methods with the CAR detection reagent, and antibodies against CD3, CD4 and CD8. Dead cell exclusion was performed using 7-aminoactinomycin D (7-AAD).

Interferon γ and IL-2 ELISAs

One-hundred thousand target cells were combined with 100,000 CAR-transduced T cells in duplicate wells of a 96 well round bottom plate in 200 μL of AIMN-V medium+5% human serum. The plates were incubated at 37° C. for 18-20 hours. Following the incubation, ELISAs for interferon gamma (IFNγ) and interleukin-2 were performed by using standard methods with commercial kits (R&D, Minneapolis, MN, USA).

CD107a Assay

For each T cell culture that was tested, two tubes were prepared. One tube contained target cells expressing CD19 and/or CD20, and the other tube contained NGFR-K562 cells that are negative for CD19 and CD20. All tubes contained CAR-transduced T cells or untransduced T cells, 1 ml of AIM-V medium+5% human AB serum, a titrated concentration of an anti-CD107a antibody (Thermo, Waltham, MA, USA), and 1 μL of Golgi Stop (monesin, BD Biosciences, San Jose, CA, USA). All tubes were incubated at 37° C. for 4 hours and then stained for CD3, CD4, and CD8.

Flow Cytometry

Flow cytometry analysis for all experiments was performed by using FlowJo (Tree Star, Inc., Ashland, OR, USA).

Proliferation Assays

Cocultures were set up in 24-well plates. Target cells included in cocultures were either 0.5×10⁶ irradiated CD19-K562 cells, 0.5×10⁶ irradiated CD20-K562 cells, or 0.5×10⁶ irradiated NGFR-K562 cells. The cocultures also included 1×10⁶ T cells from cultures that had been transduced with either MSGV1-Hu1928-Hu20BB std 10-5-2020 or MSGV1-Hu1928-Hu20BB long 10-21-2020. The T cells were labeled with carboxyfluorescein diacetate succinimidyl ester (CFSE, Invitrogen) as previously described (Mannering et al., Journal of Immunological Methods, 283: 173-183 (2003), incorporated by reference herein). The medium used in the cocultures was AIM V⁺5% human AB serum. IL-2 was not added to the medium. Four days after initiation, the live cells in each coculture were counted with trypan blue for dead cell exclusion, and flow cytometry was performed.

Cytotoxicity Assay

Cytotoxicity assays were conducted as previously described (Kochenderfer et al., Journal of Immunotherapy, 32: 689-702 (2009), incorporated by reference herein). Cytotoxicity was measured by comparing survival of primary chronic lymphocytic leukemia target cells relative to the survival of negative-control CCRF-CEM cells. Both cell types were combined in the same tubes with CAR-transduced T cells. CCRF-CEM negative control cells were labeled with the fluorescent dye 5-(and-6)-(((4-chloromethyl)benzoyl)amino) tetramethylrhodamine (CMTMR) (Invitrogen), and primary CLL target cells were labeled with CFSE. Cocultures were set up in sterile 5 mL test tubes (BD) in duplicate at multiple T cell to target-cell ratios. The target cells contained in the tubes were 50,000 CLL target cells along with 50,000 CCRF-CEM negative-control cells. The cultures were incubated for 4 hours at 37° C. Immediately after the incubation, 7AAD (7-amino-actinomycin D) (BD) was added, and flow cytometry acquisition was performed. For each T cell plus target-cell culture, the percent survival of CLL target cells was determined by dividing the percent live CLL cells by the percent live CCRF-CEM negative control cells. The corrected percent survival of CLL target cells was calculated by dividing the percent survival of CLL target cells in each T cell plus target cell culture by the ratio of the percent live CLL target cells to percent live CCRF-CEM negative-control cells in tubes containing only CLL target cells and CCRF-CEM cells without effector T cells. This correction was used to account for variation in the starting cell numbers and for spontaneous target cell death. Cytotoxicity was calculated as follows: the percent cytotoxicity of CLL target cells=100-corrected percent survival of CLL target cells.

Mouse Tumor Experiments

NOD.Cg-Prkdc^{scid}I2rg^{tm1wjI}/SzJ (NSG) mice at 6-8 weeks of age from NCI-Frederick or the Jackson Laboratories were injected with one of four types of human tumor cell line cells. Tumors were allowed to grow until measurable tumors were present. All tumor cell line cells were injected intradermally in a 1:1 mix of Matrigel and PBS. For st486 cells, 4×10⁶ cells were injected and allowed to grow for 6 days prior to CAR T-cell injection. For MM.1 S cells, 4×10⁶ cells were injected and allowed to grow for 7 days prior to CAR T-cell injection. For NALM6 cells, 4×10⁶ cells were injected and allowed to grow for 6 days prior to CAR T-cell injection. For st486-CD19neg, 4×10⁶ cells were injected and allowed to grow for 6 days prior to CAR T-cell injection. CAR T cells that had been started in culture 7 days prior to injection were injected intravenously at the CD3⁺ CAR⁺ cells/mouse doses indicated in figure legends. Mice received 1 injection of CAR T cells. Tumors were measured using a caliper every three days, and the volume of the tumors were calculated using the formula (length×width×height)/2. Mice were sacrificed once tumors reached 15 mm in the longest length.

### Example 1

This example illustrates recombination events in previous bicistronic CAR constructs.

Hu1928-Hu20BB-original encodes the fully-human CARs Hul9-CD828Z and Hu20-CD8BBZ (WO 2020/061048, incorporated by reference herein). Hul9-CD828Z included an anti-CD19 single-chain variable fragment (scFv), a CD8α hinge and transmembrane domain, a CD28 costimulatory domain, and a CD3ζ T-cell activation domain. Hu20-CD8BBZ included an anti-CD20 scFv, a CD8α hinge and transmembrane domain, a 4-1BB costimulatory domain, and a CD3ζ T-cell activation domain.

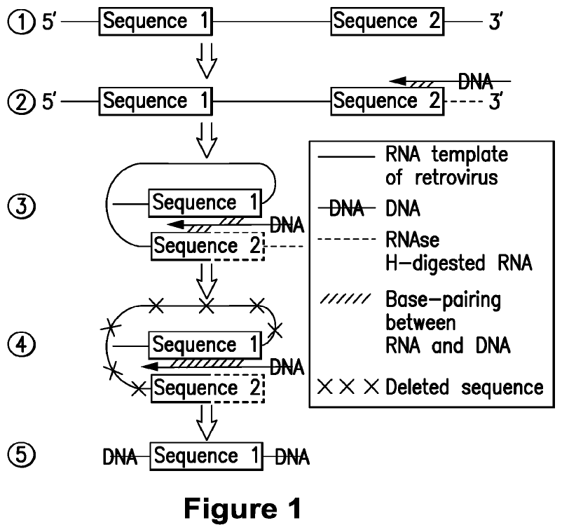

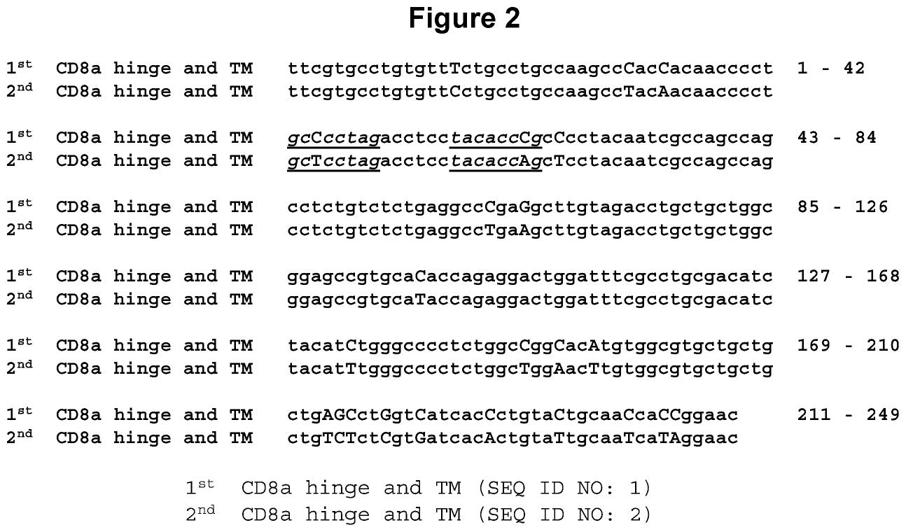

When T cells were transduced with gamma-retroviruses encoding Hu1928-Hu20BB-original, apparent recombination events occurred that led to deletion of parts of the expected CAR sequences in a minority of the RNA transcripts from the transduced T cells. Without wishing to be bound by theory, FIG. 1 diagrammatically presents a possible mechanism of recombination. These deletions in the expected CAR sequences were determined by RNA sequencing. An example of regions of identical sequence in different areas of the Hu1928-Hu20BB- original construct is shown in FIG. 2 by aligning the nucleotide sequence of the CD8α hinge and transmembrane domain of the Hul9-CD828Z CAR of this construct with the nucleotide sequence of the Hu20-CD8BBZ CAR of the same construct. This alignment shows many regions of identical nucleotide sequence in these CD8α hinge and transmembrane domain sequences that could promote retroviral recombination leading to deletion of portions of the intended final proteins encoded by this construct. Examples of the two common aberrant RNA products resulting from apparent retroviral recombination events involving areas of identical nucleotide sequence shared between the CD8α hinge and transmembrane domains of the two CARs contained in the Hu1928-Hu20BB-original construct are shown in Table 10.

TABLE 10

| | Location of last | | | | |
|---|---|---|---|---|---|
| | nucleotide | Nucleotide | Nucleotide | | |
| | prior to deletion in | sequence | sequence | | |
| Isoform | the 1^{st} CD8a hinge | just before | just after | | |
| identifying | and transmembrane | deletion | deletion | | Resulting RNA |
| number* | domain* | start | end | Sequence deleted | expressed |
| 20161.145 | Nucleotide 50 | gcccctag | acctccta | Hu19-CD828Z: some | A CAR with CD8a |
| | | | | nucleotides of | signal sequence, |
| | | | | CD8a hinge and | Hu19 scFv, CD8a |
| | | | | transmembrane domain, | hinge and |
| | | | | all nucleotides of CD28 | transmembrane |
| | | | | and CD3z. Hu20- | domain, CD28, and |
| | | | | CD8BBZ: all nucleotides | CD3z. All |
| | | | | of GM-CSF receptor | components are |
| | | | | signal sequence, Hu20 | complete and form a |
| | | | | scFv, and some | CAR sequence that |
| | | | | nucleotides of CD8a | could be expressed. |
| | | | | hinge and transmembrane | |
| | | | | domain. | |
| 20161.26 | Nucleotide 64 | tacacccg | ctcctaca | Hu19-CD828Z: some | A CAR with CD8a |
| | | | | nucleotides of | signal sequence, |
| | | | | CD8a hinge and | Hu19 scFv, CD8a |
| | | | | transmembrane domain, | hinge and |
| | | | | all nucleotides of CD28 | transmembrane |
| | | | | and CD3z. Hu20- | domain, CD28, and |
| | | | | CD8BBZ: all nucleotides | CD3z. All |
| | | | | of GM-CSF receptor | components are |
| | | | | signal sequence, Hu20 | complete and form a |
| | | | | scFv, and some | CAR sequence that |
| | | | | nucleotides of CD8a | could be expressed. |
| | | | | hinge and transmembrane | |
| | | | | domain. | |

^{∧}Deletion events were determined by RNA sequencing by Illumina short sequence RNAseq and single-molecule, real-time PacBio RNA sequencing.
*This column refers to the last nucleotide of the CD8a hinge and transmembrane domain 5′ to the deleted sequence.

### Example 2

This Example demonstrates designs of the inventive CARs, in accordance with aspects of the disclosure.

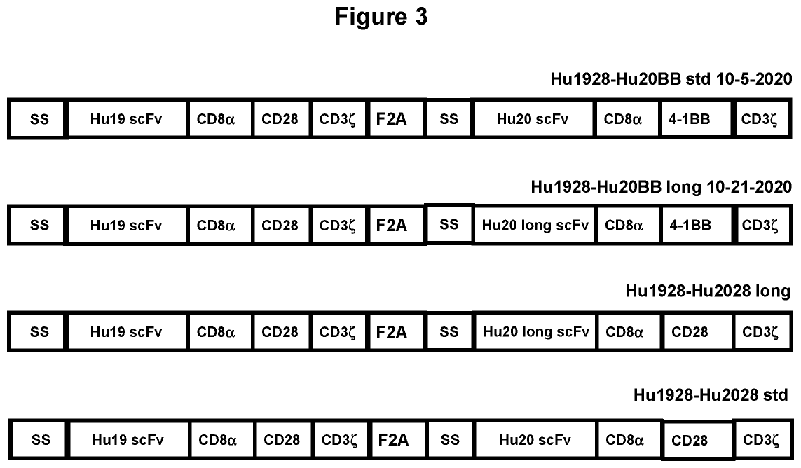

CAR constructs were designed that are different than the previously-reported Hu1928-Hu20BB-original. First, regions of identical DNA sequences in different components of the CAR constructs have been greatly reduced to reduce the risk of retroviral recombination events. Second, a different signal sequence from the GM-CSF receptor has been adopted for the new Hu20-CD8BBZ CAR. Third, the linker in the Hu20 scFv has been lengthened in some versions. Fourth, versions with a Hu20-containing CAR containing a CD28 costimulatory domain instead of a 4-1BB costimulatory domain have been designed. The bicistronic CAR constructs incorporating these design features are Hu1928-Hu20BB std 10-5-2020, Hu1928-Hu20BB long 10-21-2020, Hu1928-Hu2028 long, and Hu1928-Hu2028 std (FIG. 3).

As seen in FIG. 3, starting at the N-terminus, the components of constructs are: the human CD8α signal sequence (first SS), the Hul9 anti-CD19 scFv, the human CD8α hinge and transmembrane sequence, the cytoplasmic portion of human CD28, the cytoplasmic portion of human CD3ζ, a F2A ribosomal skip sequence, the GM-CSF receptor signal sequence (second SS), the Hu20 anti-CD20 scFv, the human CD8α hinge and transmembrane sequence, the cytoplasmic portion of human 4-1BB or CD28, and the cytoplasmic portion of CD3ζ. All variants have a sequence designed to reduce overlapping areas of identical sequence to reduce the risk of recombination. For Hu1928-Hu20BB std 10-5-2020, this CAR has a Hu20 scFv linker of (G4S)3 linker, which is 3 replicates of amino acids GGGGS (SEQ ID NO: 70). Hu1928-Hu20BB long 10-21-2020 is identical to Hu1928-Hu20BB std 10-5-2020 except the linker in the Hu20 scFv was lengthened to (G4S)4, which is 4 replicates of amino acids GGGGS (SEQ ID NO: 70). Hu1928-Hu2028 long is identical to Hu1928-Hu20BB long 10-21-2020 except for the replacement of the 4-1BB domain with a CD28 domain. Hu1928-Hu2028 std has the same nucleotide and amino acid sequences as Hu1928-Hu2028 long except the (G4S)4 long linker has been shortened to the (G4S)3 std linker.

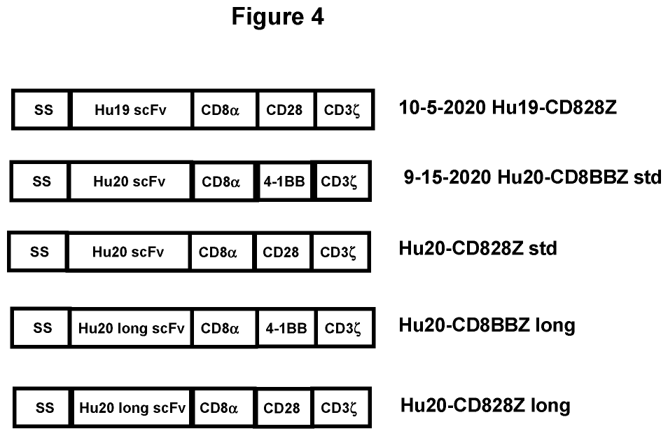

Monospecific versions of the CARs included in these bicistronic constructs include: 10-5-2020 Hul9-CD828Z, 9-15-2020 Hu20-CD8BBZ std, Hu20-CD828Z std, Hu20-CD8BBZ long, Hu20-CD828Z long (FIG. 4).

As seen in FIG. 4, starting at the N-terminus, the components of 10-5-2020 Hul9-CD828Z are: the human CD8α signal sequence, the Hul9 anti-CD19 scFv, the human CD8a hinge and transmembrane sequence, the cytoplasmic portion of human CD28, the cytoplasmic portion of human CD3ζ. The DNA sequence of 10-5-2020 Hul9-CD828Z was designed to reduce areas of overlapping DNA sequence with Hu20-CD8BBZ. Starting at the N-terminus, the components of 9-15-2020 Hu20-CD8BBZ std are: the human GM-CSF receptor signal sequence, the Hu20 anti-CD20 scFv, the human CD8α hinge and transmembrane sequence, the cytoplasmic portion of human 4-1BB, the cytoplasmic portion of human CD3ζ Hu20-CD828Z std has the same sequence as 9-15-2020 Hul9-CD8BBZ std except that the 4-1BB sequence has been replaced with a CD28 sequence. Hu20-CD8BBZ long is identical to 9-15-2020 Hu20-CD8BBZ std except that the linker of the Hu20 scFv was lengthened from 3 GGGGS amino acid replicates in 9-15-2020 Hu20-CD8BBZ std to 4 GGGGS replicates in Hu20-CD8BBZ long. Hu20-CD828Z long is identical to Hu20-CD8BBZ long except that the 4-1BB moiety has been replaced by a CD28 moiety. In the construct names, std designates a standard Hu20 scFv linker of (G4S)3, and long designates a lengthened Hu20 scFv amino acid sequence of (G4S)4.

The bicistronic CAR constructs Hu1928-Hu20BB std 10-5-2020, Hu1928-Hu20BB long 10-21-2020, Hu1928-Hu2028 long, and Hu1928-Hu2028 std all share the same nucleotide sequences in the following components: CD8α signal sequences, GM-CSF receptor signal sequences, Hul9 scFvs, Hu20 light chain variable regions, and Hu20 heavy chain variable regions. The CD28 moiety of the Hul9-CD828Z CARs have the same nucleotide sequences in each bicistronic CAR construct. The 4-1BB moieties of the Hu20-CD8BBZ CARs of Hu1928-Hu20BB std 10-5-2020 and Hu1928-Hu20BB long 10-21-2020 have the same nucleotide sequences. The monospecific CARs (FIG. 4) are all components of the bicistronic CAR constructs (FIG. 3). Each monospecific CAR has a nucleotide sequence identical to the nucleotide sequence of the same CAR when it is included in the bicistronic CAR constructs. The monospecific CAR names are shortened in the bicistronic CAR construct designations: Hul9-CD828Z is shortened to Hu1928; Hu20-CD8BBZ is shortened to Hu20BB; Hu20-CD828Z is shortened to Hu2028.

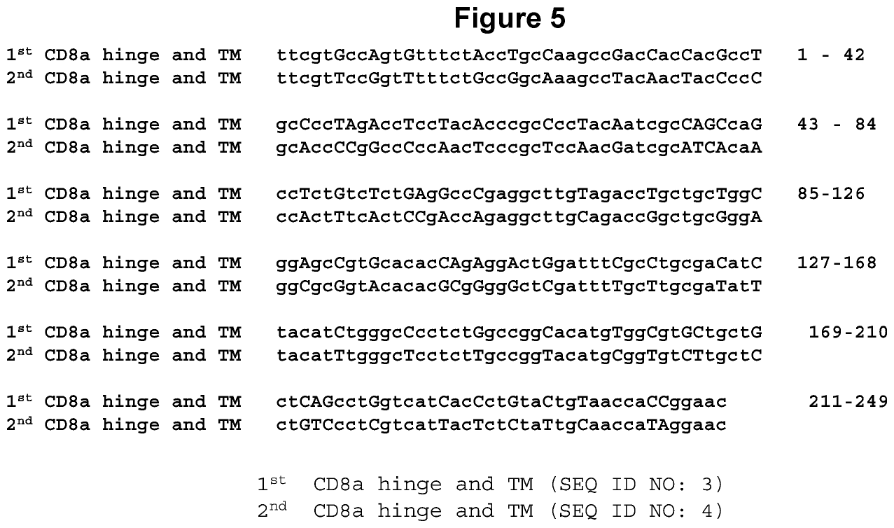

Among other nucleotide changes, Hu1928-Hu20BB-original was changed by incorporating CD8α hinge and transmembrane domain nucleotide sequences with greatly reduced regions of identical nucleotide sequence shared by the first and second CD8α hinge and transmembrane domains of the construct in the new Hu1928-Hu20BB std (standard) 10-5-2020 CAR construct (FIGS. 2 and 5). Nucleotide changes were made throughout all regions of the Hu1928-Hu20BB-original construct to reduce nucleotide identity where there were two areas with identical nucleotide sequence. Reducing regions of identical nucleotide sequence in the Hu1928-Hu20BB-original construct led to reduced incidence of deletions of intended RNA sequences, which were presumably caused by homology-driven retroviral recombination events. The reduction in incidence of deletions due to recombination events is summarized for different CAR regions in Table 11.

Table 11 shows the the fraction of total RNA transcripts that included deletions presumably due to recombination events. The fraction of total RNA transcripts with unexpected deletions is shown for the major CAR domains of Hu1928-Hu20BB-original and Hu1928-Hu20BB std 10-5-2020. The fraction of transcripts with deletions is much lower for the Hu1928-Hu20BB std 10-5-2020 CAR versus Hu1928-Hu20BB-original.

TABLE 11

| | Hu1928-Hu20BB- | Hu1928-Hu20BB std |
|---|---|---|
| CAR domain | original | Oct. 5, 2020 |
| Hu19 light chain domain | 0.0118 | 0 |
| Hu19 heavy chain domain | 0.0279 | 0.0009 |
| 1st CD8a hinge and | 0.2442 | 0 |
| transmembrane domain | | |
| CD28 | 0 | 0 |
| 1st CD3z | 0.0022 | 0.0009 |
| Hu20 light chain domain | 0.0022 | 0 |
| Hu20 heavy chain domain | 0.0022 | 0 |
| 2nd CD8a hinge and | 0.0029 | 0.0027 |
| transmembrane domain | | |
| 4-1BB | 0.0066 | 0 |
| 2nd CD3z | 0 | 0 |

For Table 11, cells from the same donor were transduced with transiently-produced MSGV1 vectors encoding the indicated CAR constructs. RNA was analyzed by single-molecule real-time (SMRT) RNAseq analysis. Values are fractions of total transcripts that had deletions of the expected sequences from the indicated CAR domains. The values are the sum of all deletions with 5-prime ends in the indicated regions. Only deletions occurring in at least 3 transcripts are included; deletions detected in less than 3 transcripts are recorded as zero.

### Example 3

This Example demonstrates expression of the inventive CARs, in accordance with aspects of the disclosure.

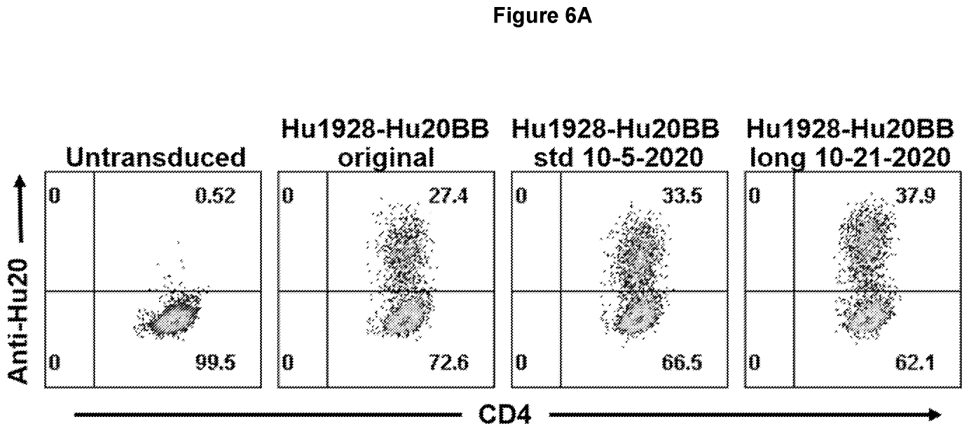

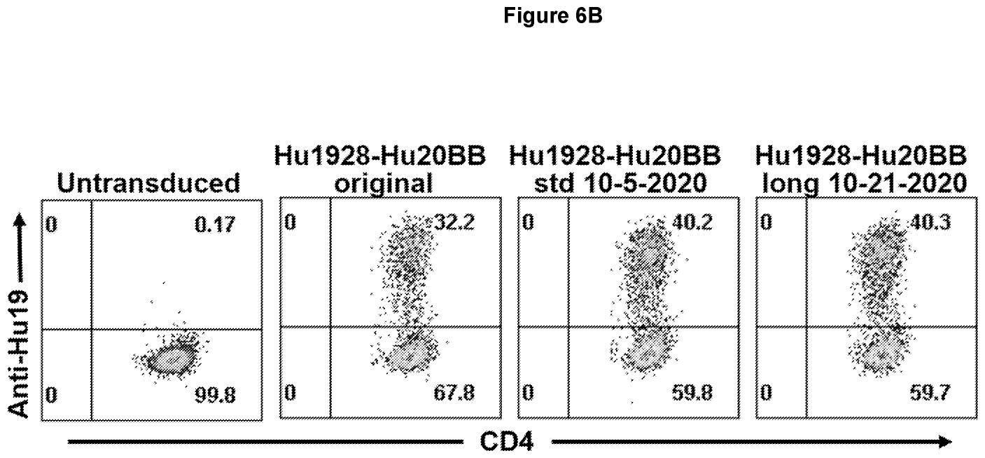

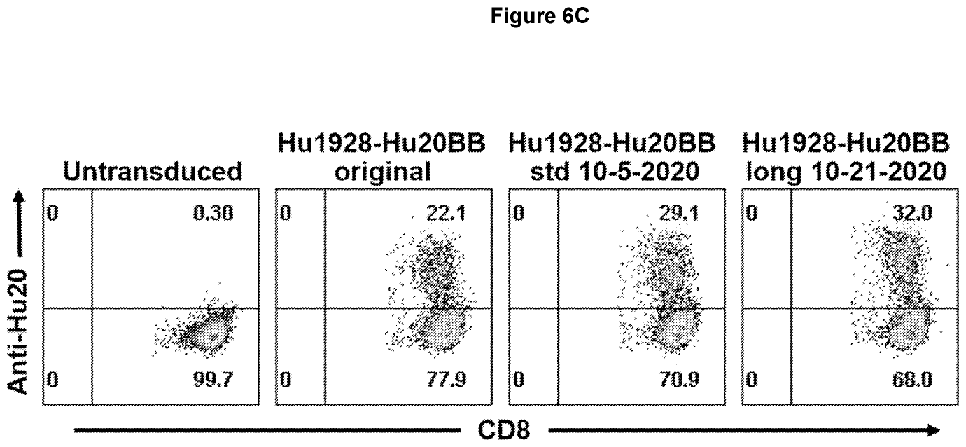

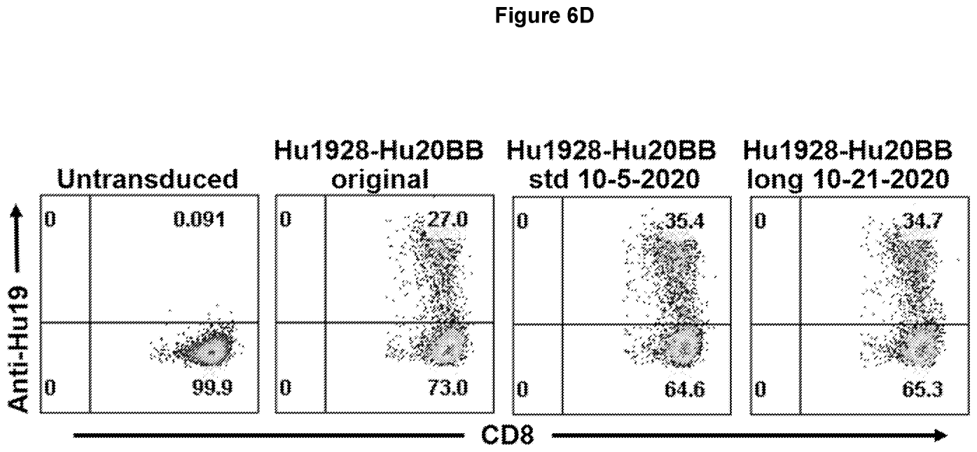

All experiments utilized primary human T cells transduced with the various CAR constructs or left untransduced. Human donor T cells from the same human donor were transduced with MSGV1 gamma-retroviral vectors encoding Hu1928-Hu20BB-original, Hu1928-Hu20BB std 10-5-2020, or Hu1928-Hu20BB long 10-21-2020. Expression of the Hul9-CD828Z and Hu20-CD8BBZ CARs encoded by these constructs were assessed 5 days after transduction on both CD4⁺ and CD8⁺ T cells, as follows. Human PBMC from the same donor were placed in culture with medium containing an anti-CD3 antibody and IL-2. On day 2 of culture, the cells were left untransduced, or they were transduced with MSGV1 gamma-retroviral vectors encoding one of three CARs: Hu1928-Hu20BB- original, Hu1928-Hu20BB std 10-5-2020, and Hu1928-Hu20BB long 10-21-2020. Five days after transduction, flow cytometry was performed (FIGS. 6A-6D). All plots were gated on live, CD3⁺ lymphocytes. Cells were also stained with a monoclonal antibody that specifically bound the Hul9 scFv and a different antibody that specifically bound the Hu20 scFv.

For cells transduced with MSGV1-Hu1928-Hu20BB std 10-5-2020 and MSGV1-Hu1928-Hu20BB long 10-21-2020, expression of Hul9-CD828Z and Hu20-CD8BBZ was higher when compared to expression of these CARs by T cells transduced with MSGV1-Hu1928-Hu20BB-original.

### Example 4

This Example demonstrates inventive CAR T-cell degranulation, in accordance with aspects of the disclosure.

Degranulation was assessed by measuring expression of CD107a on T cells after culture with target cells. T cells were cultured and transduced as described in Example 3. On day 7, the cells were cultured in the presence of an antibody against CD107a for 4 hours with one of the following target cells: CD19-K562, CD20-K562, st486, or NGFR-K562. The cells were then stained with antibodies against CD3, CD4, and CD8.

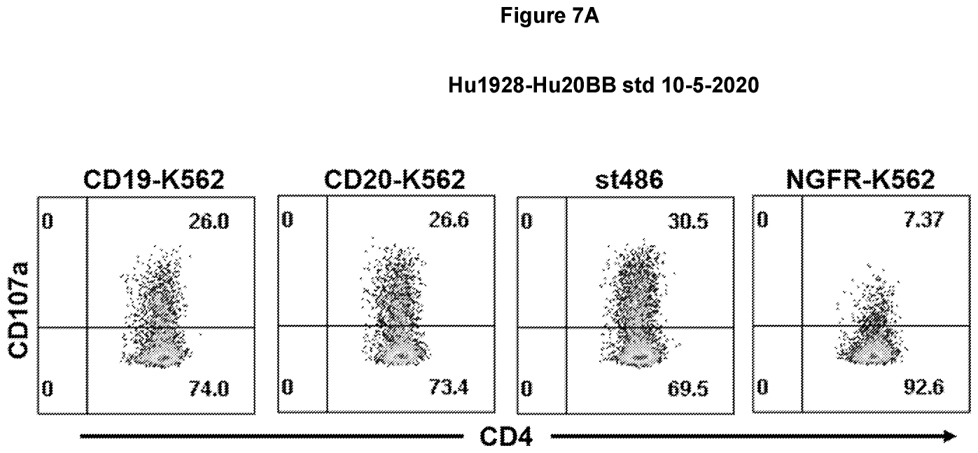

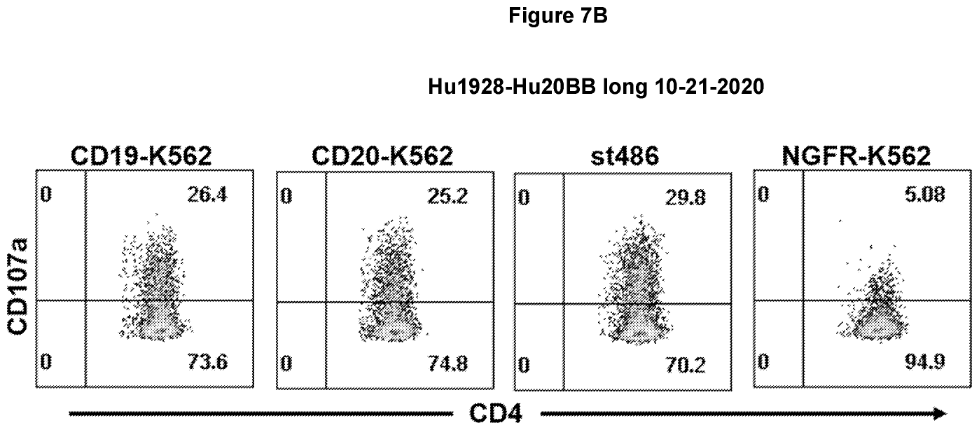

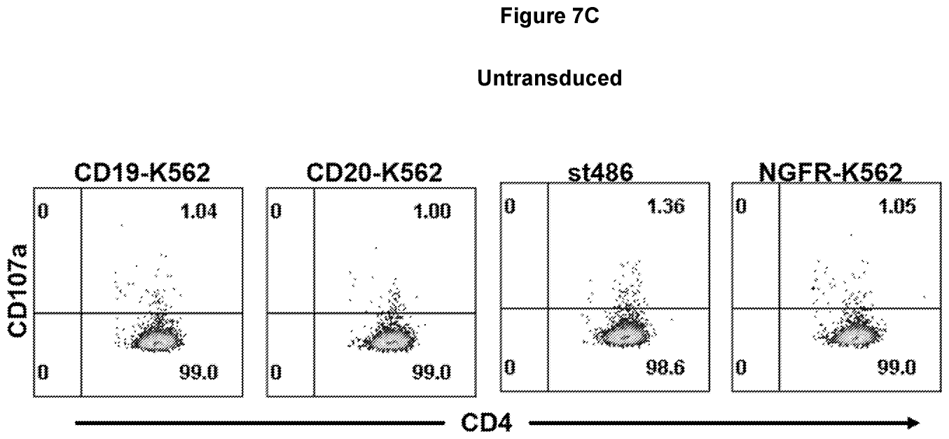

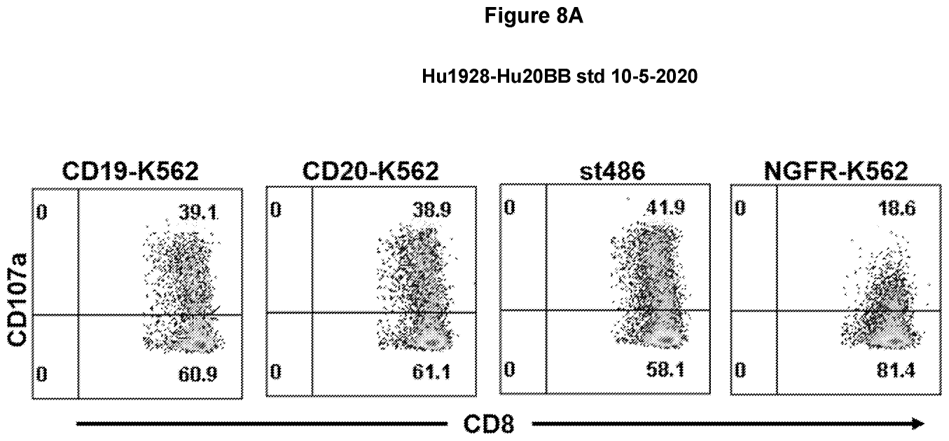

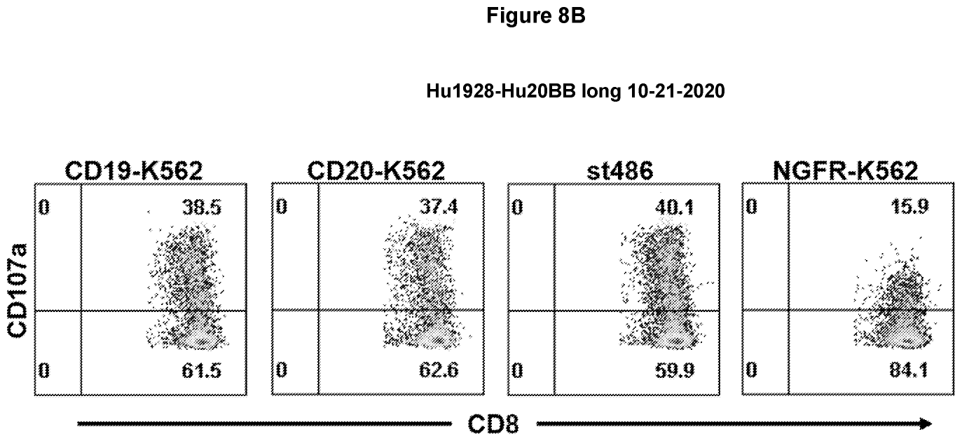

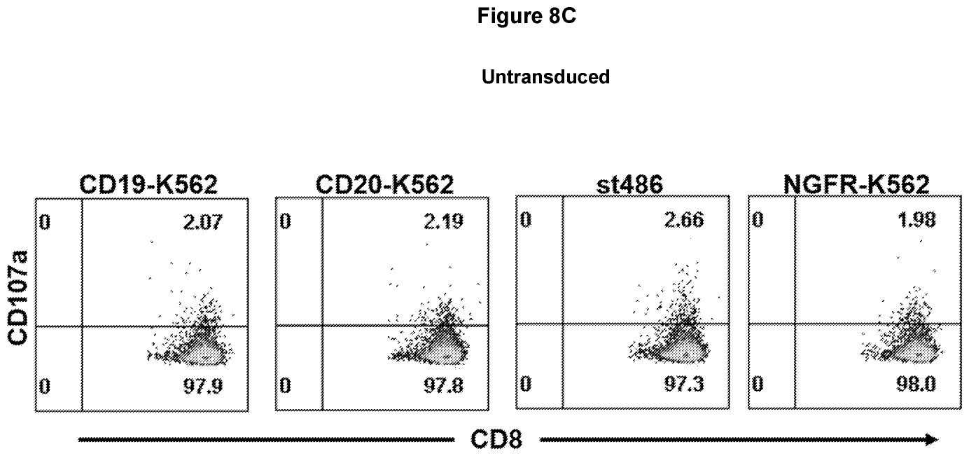

It was found that both CD4⁺ and CD8⁺ T cells expressing either Hu1928-Hu20BB std 10-5-2020 or Hu1928-Hu20BB long 10-21-2020 degranulated in response to target cells expressing CD19 (CD19-K562), CD20 (CD20-K562), or both CD19 and CD20 (st486) (FIGS. 7A-7C and 8A-8C). Degranulation was detected at lower levels when the transduced T cells were cultured with NGFR-K562 target cells that express neither CD19 nor CD20. Untransduced T cells exhibited only low levels of degranulation.

### Example 5

This Example demonstrates CAR T-cell cytokine release, in accordance with aspects of the disclosure.

T cells from a human donor were transduced with gamma-retroviruses encoding the CAR constructs or left untransduced. Seven days after transduction, the T cells were cultured alone or with the indicated target cells as indicated overnight. After the overnight culture, and IFNγ and IL-2 ELISA were performed on the culture supernatant.

It was found that T cells expressing either Hu1928-Hu20BB std 10-5-2020 or Hu1928-Hu20BB long 10-21-2020 released IFNγ (Tables 12A-12C) and IL-2 (Tables 13A-13C) in an antigen-specific manner. For Tables 12A-12C and 13A-13D: all values are pg/mL of IFNγ/IL-2 except for the % CAR⁺; % CAR⁺ indicates the percentage of transduced T cells that expressed both Hul9-CD828Z and Hu20-CD8BBZ.

TABLE 12A

| | | | | st486 CD19- |
|---|---|---|---|---|
| | CD19-K562 | CD20-K562 | st486 | negative |
| Untransduced | 29.3 | 23.5 | 153.0 | 167.5 |
| Hu1928- | 9290.9 | 16155.0 | 7500.6 | 7265.9 |
| Hu20BB std | | | | |
| Oct. 5, 2020 | | | | |
| Hu1928- | 8213.8 | 20475.5 | 7765.4 | 7672.7 |
| Hu20BB long | | | | |
| Oct. 21, 2020 | | | | |

TABLE 12B

| | | Toledo CD19- | | |
|---|---|---|---|---|
| | Toledo | negative | CEM | NGFR-K562 |
| Untransduced | 51.6 | 29.3 | 16.5 | 30.1 |
| Hu1928- | 8366.6 | 5888.8 | 62.6 | 86.4 |
| Hu20BB std | | | | |
| Oct. 5, 2020 | | | | |
| Hu1928- | 8021.9 | 6686.9 | 22.9 | 42.3 |
| Hu20BB long | | | | |
| Oct. 21, 2020 | | | | |

TABLE 12C

| | T cells Alone | % CAR+ |
|---|---|---|
| Untransduced | 12.6 | 0.0 |
| Hu1928- | 63.2 | 31.5 |
| Hu20BB std | | |
| Oct. 5, 2020 | | |
| Hu1928- | 21.0 | 34.8 |
| Hu20BB long | | |
| Oct. 21, 2020 | | |

TABLE 13A

| | | | | ST486 CD19- |
|---|---|---|---|---|
| | CD19-K562 | CD20-K562 | ST486 | negative |
| Untransduced | <15.6 | <15.6 | 58.3 | 61.8 |
| Hu1928- | 179.5 | 591.3 | 333.9 | 241.7 |
| Hu20BB std | | | | |
| Oct. 5, 2020 | | | | |
| Hu1928- | 495.7 | 2286.3 | 1250.8 | 1220.2 |
| Hu20BB long | | | | |
| Oct. 21, 2020 | | | | |

TABLE 13B

| | | Toledo CD19- | | |
|---|---|---|---|---|
| | Toledo | negative | CEM | NGFR-K562 |
| Untransduced | 23.4 | <15.6 | <15.6 | <15.6 |
| Hu1928- | 426.2 | 116.7 | <15.6 | <15.6 |
| Hu20BB std | | | | |
| Oct. 5, 2020 | | | | |
| Hu1928- | 1400.1 | 674.7 | <15.6 | <15.6 |
| Hu20BB long | | | | |
| Oct. 21, 2020 | | | | |

TABLE 13C

| | T cells Alone | % CAR+ |
|---|---|---|
| Untransduced | <15.6 | 0.0 |
| Hu1928- | <15.6 | 31.5 |
| Hu20BB std | | |
| Oct. 5, 2020 | | |
| Hu1928- | <15.6 | 34.8 |
| Hu20BB long | | |
| Oct. 21, 2020 | | |

T cells expressing Hu1928-Hu20BB long 10-21-2020 released higher levels of IL-2 when compared with T cells expressing Hu1928-Hu20BB std 10-5-2020; this result was obtained in four of four experiments with different donors. The demonstration of antigen-specific IFNγ release by Hu1928-Hu20BB std 10-5-2020 and Hu1928-Hu20BB long 10-21-2020 was repeated, and the expression of 10-5-2020 Hul9-CD828Z and the ability of this CAR to release IFNγ in an antigen-specific manner were demonstrated (Tables 14A-14C). For Tables 14A-14C: % CAR⁺ indicates the percentage of transduced T cells that expressed both Hul9-CD828Z and Hu20-CD8BBZ for bicistronic constructs or just Hul9-CD828Z for the monospecific 10-5-2020 Hul9-CD828Z construct.

TABLE 14A

| | CD19- | CD20- | | ST486 CD19- |
|---|---|---|---|---|
| | K562 | K562 | ST486 | negative |
| Untransduced | 217.2 | 201.3 | 1674.8 | 1464.3 |
| Oct. 5, 2020 Hu19- | 22046.2 | 290.6 | 2067.8 | 1360.7 |
| CD828Z | | | | |
| Hu1928-Hu20BB std | 29071.5 | 41357.7 | 11165.9 | 14048.3 |
| Oct. 5, 2020 | | | | |
| Hu1928-Hu20BB long | 37534.4 | 60870.8 | 16527.1 | 19223.4 |
| Oct. 21, 2020 | | | | |

TABLE 14B

| | | Toledo CD19- | | |
|---|---|---|---|---|
| | Toledo | negative | CEM | NGFR-K562 |
| Untransduced | 542.3 | 372.7 | 51.0 | 168.8 |
| Oct. 5, 2020 Hu19- | 3203.3 | 702.1 | 321.5 | 257.1 |
| CD828Z | | | | |
| Hu1928-Hu20BB std | 11215.9 | 7259.4 | 411.7 | 404.7 |
| Oct. 5, 2020 | | | | |
| Hu1928-Hu20BB long | 17693.8 | 7575.4 | 349.2 | 336.5 |
| Oct. 21, 2020 | | | | |

TABLE 14C

| | T cells Alone | % CAR+ |
|---|---|---|
| Untransduced | 41.3 | 0.0 |
| Oct. 5, 2020 Hu19- | 309.5 | 67.2 |
| CD828Z | | |
| Hu1928-Hu20BB std | 431.8 | 55.6 |
| Oct. 5, 2020 | | |
| Hu1928-Hu20BB long | 251.5 | 58.2 |
| Oct. 21, 2020 | | |

### Example 6

This Example demonstrates CAR T-cell proliferation, in accordance with aspects of the disclosure.

T cells expressing Hu1928-Hu20BB std 10-5-2020 or Hu1928-Hu20BB long 10-21-2020 were labelled with CFSE and cultured for 4 days with target cells expressing either CD19, CD20, or neither of these antigens. T cells were cultured and transduced as described in Example 3. On day 14 of culture, the transduced T cells were labeled with CFSE and cultured with either CD19-K562 cells, CD20-K562 cells, or NGFR-K562 cells. Four days later, the cells were stained with antibodies against CD3, CD4, and CD8.

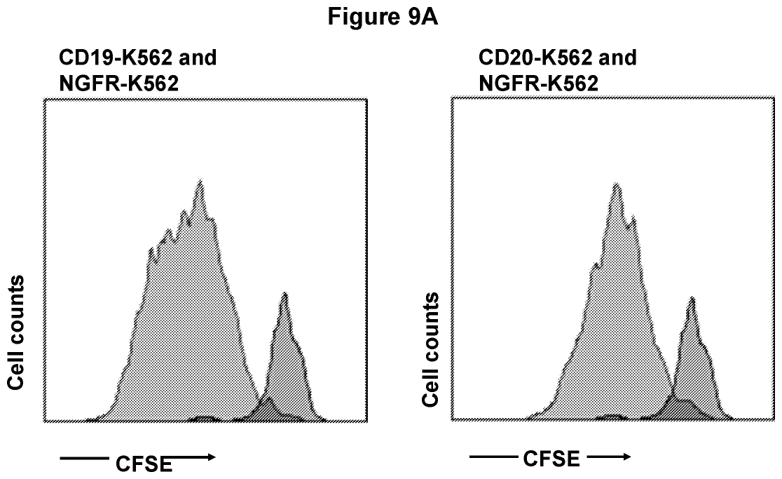

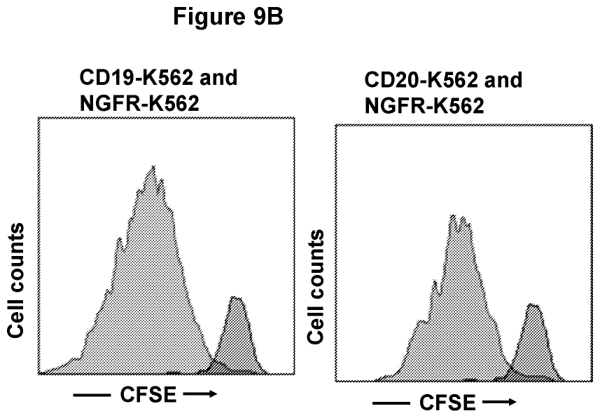

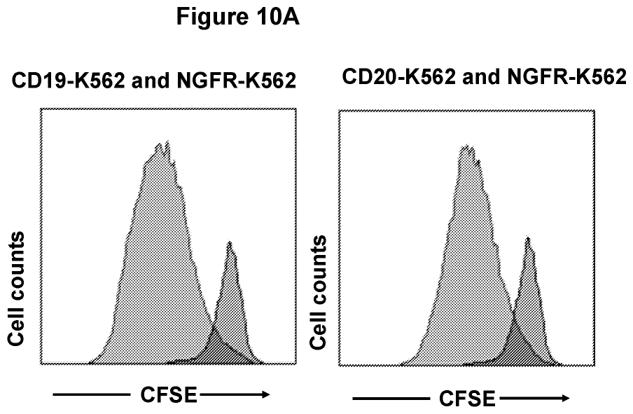

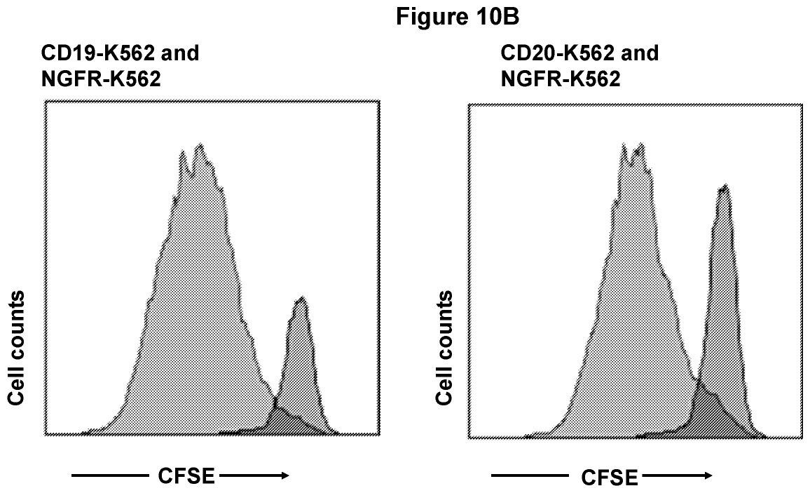

It was found that either CD4⁺ or CD8⁺ T cells expressing either of these constructs proliferated specifically in response to CD19 or CD20 (FIGS. 9 and 10). For FIG. 9A, the median fluorescence intensity for CD19-K562 was 1649 relative fluorescence units, for CD20-K562 it was 2755, and for NGFR-K562 it was 22132. For FIG. 9B, the median fluorescence intensity for CD19-K562 was 1407 relative fluorescence units, for CD20-K562 it was 2111, and for NGFR-K562 it was 28478. For FIG. 10A, the median fluorescence intensity for CD19-K562 was 1961 relative fluorescence units, for CD20-K562 it was 2871, and for NGFR-K562 it was 20520. For FIG. 10B, the median fluorescence intensity for CD19-K562 was 1771 relative fluorescence units, for CD20-K562 it was 2544, and for NGFR-K562 it was 24978.

### Example 7

This Example demonstrates expression of Hu1928-Hu2028 long and Hu1928-Hu20BB long 10-21-2020, in accordance with aspects of the disclosure.

Human PBMC from the same donor were placed in culture with medium containing an anti-CD3 antibody and IL-2. On day 2 of culture, the cells were left untransduced, or they were transduced with MSGV1 gamma-retroviral vectors encoding one of two CARs: Hu1928-Hu2028 long or Hu1928-Hu20BB long 10-21-2020. Five days after transduction, flow cytometry was performed. Cells were also stained with a monoclonal antibody that specifically bound the Hul9 scFv and a different antibody that specifically bound the Hu20 scFv.

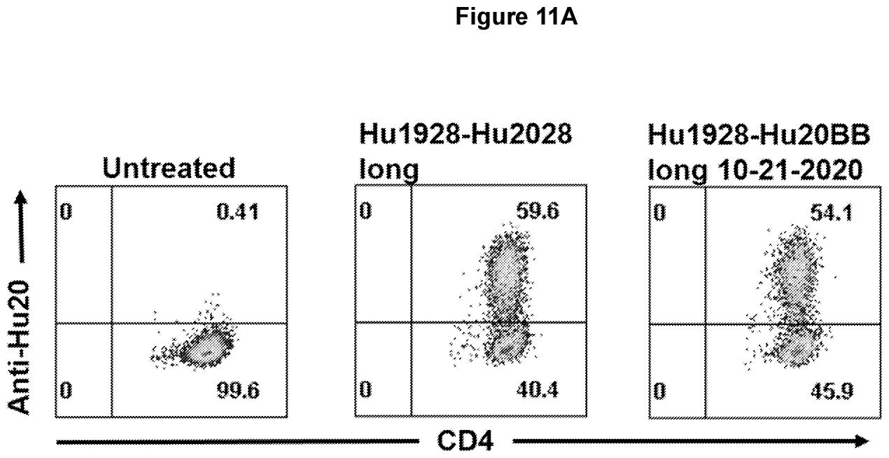

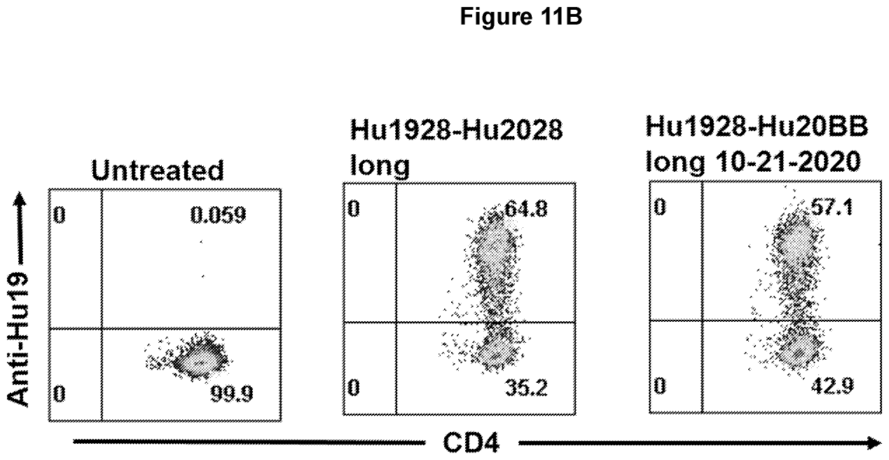

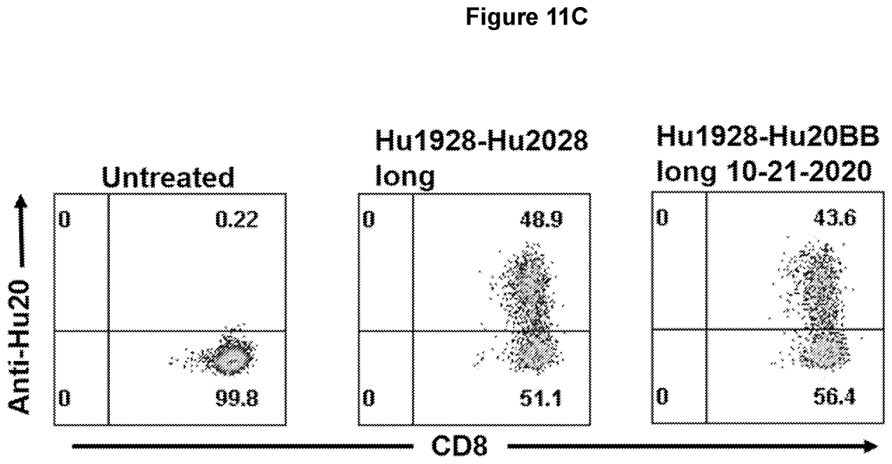

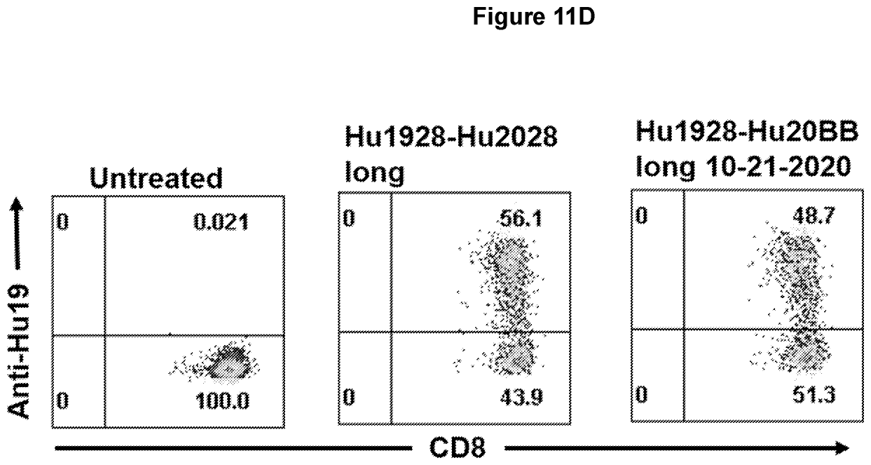

T cells that were transduced with the MSGV1-Hu1928-Hu2028 long vector expressed both the Hul9-CD828Z and Hu20-CD828Z long CARs on the T-cell surface. FIGS. 11A-11D present the results.

### Example 8

This Example demonstrates additional studies of the CARs described herein, in accordance with aspects of the disclosure.

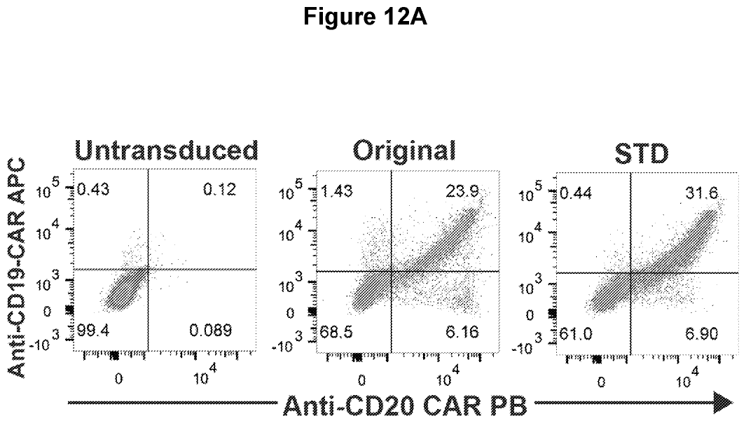

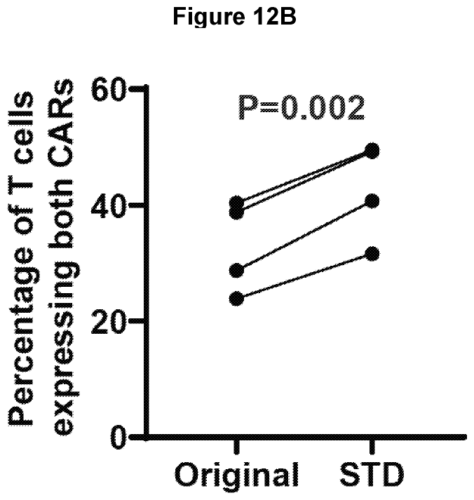

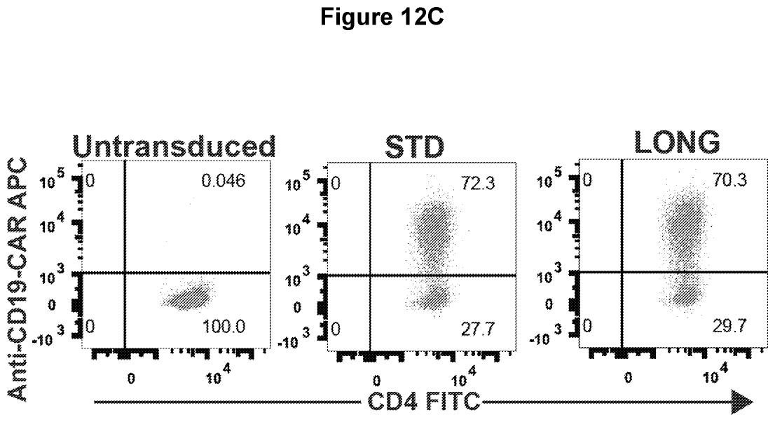

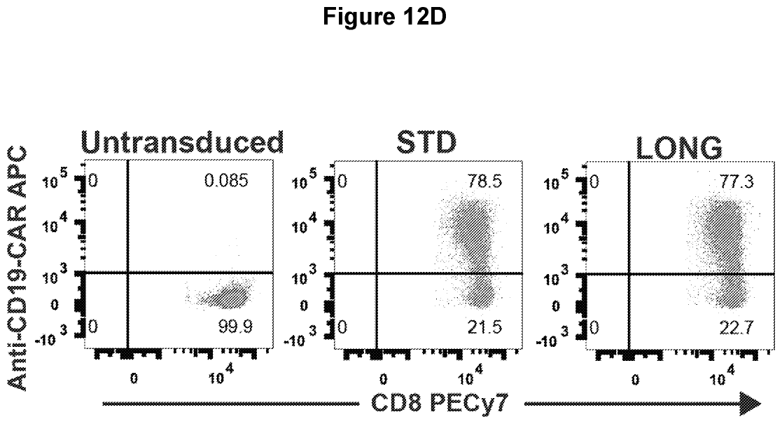

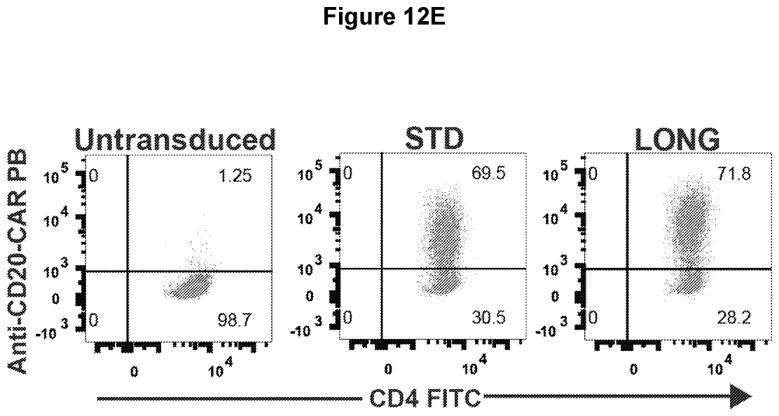

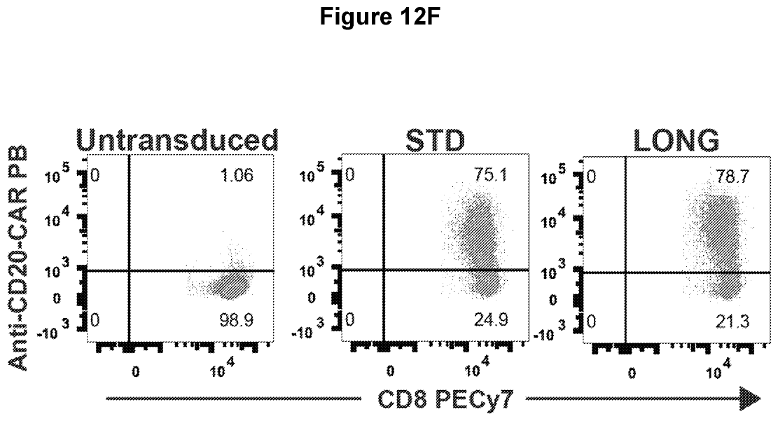

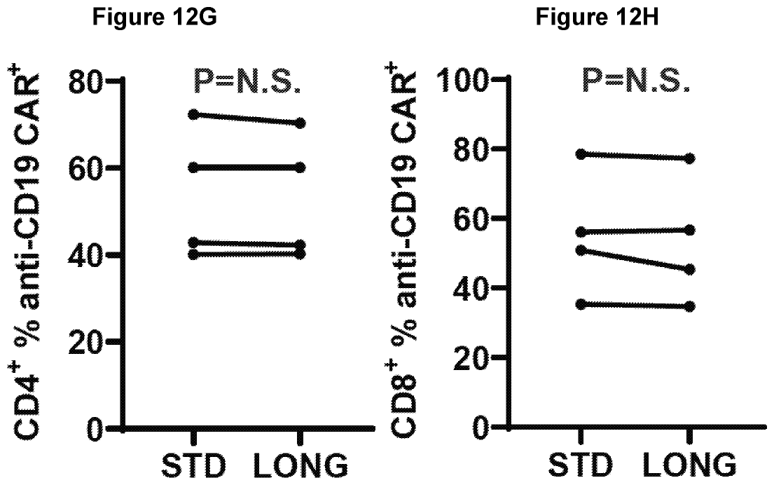

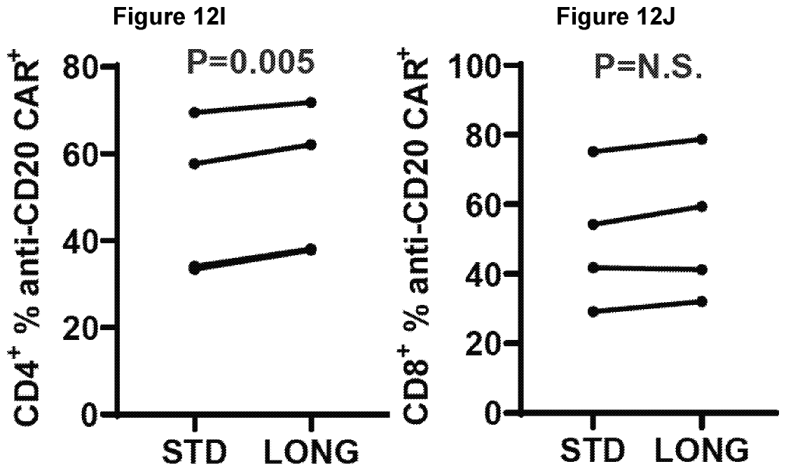

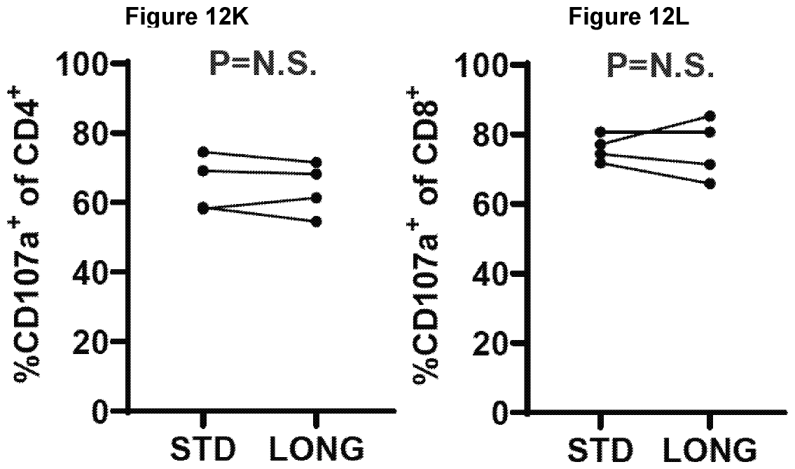

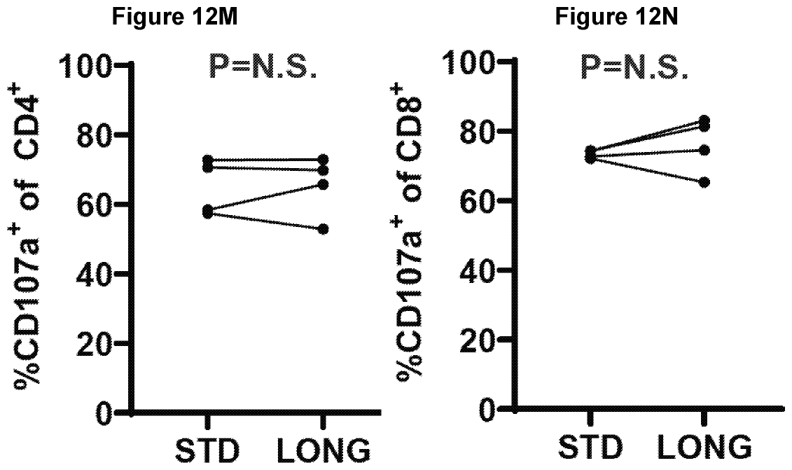

Expression of certain CARs is shown in FIGS. 12A-12N.

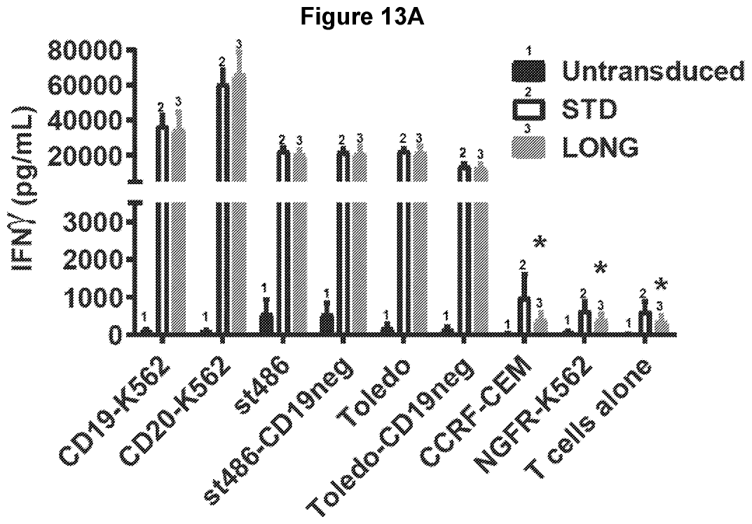

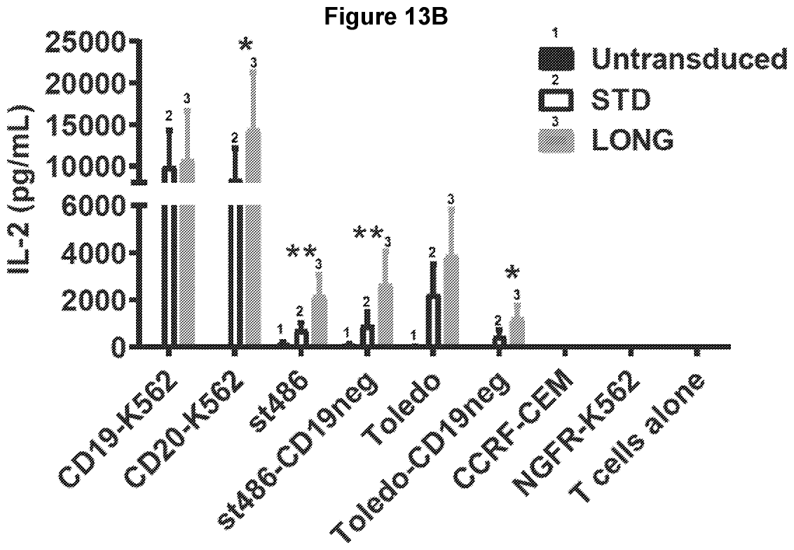

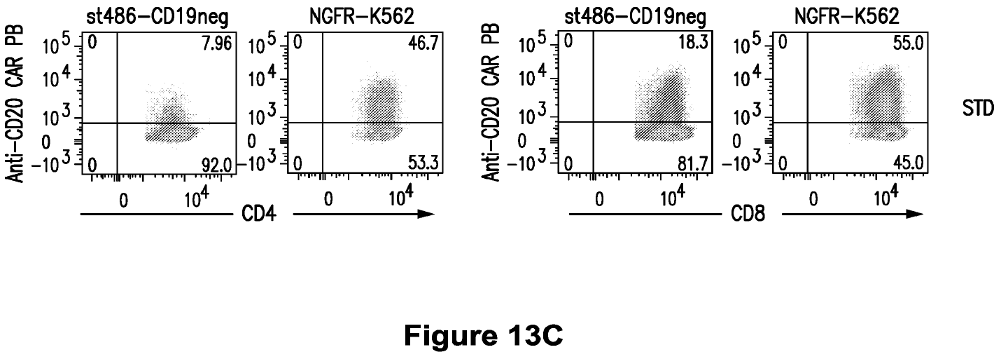

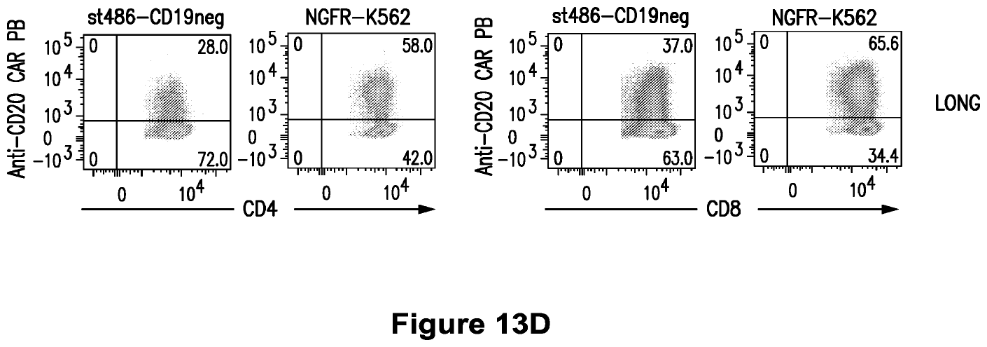

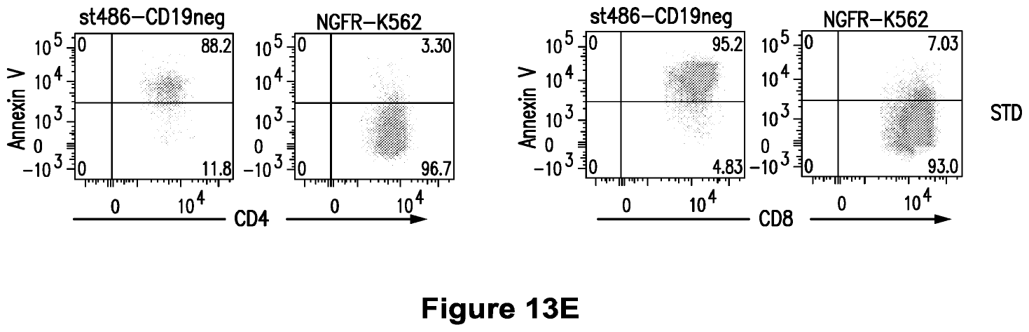

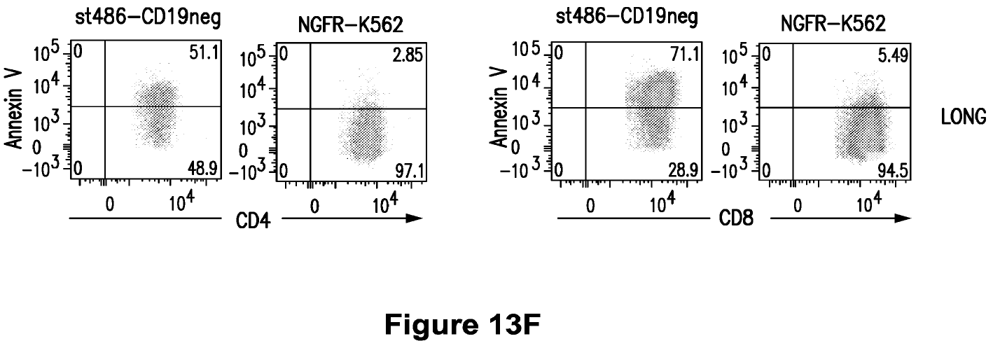

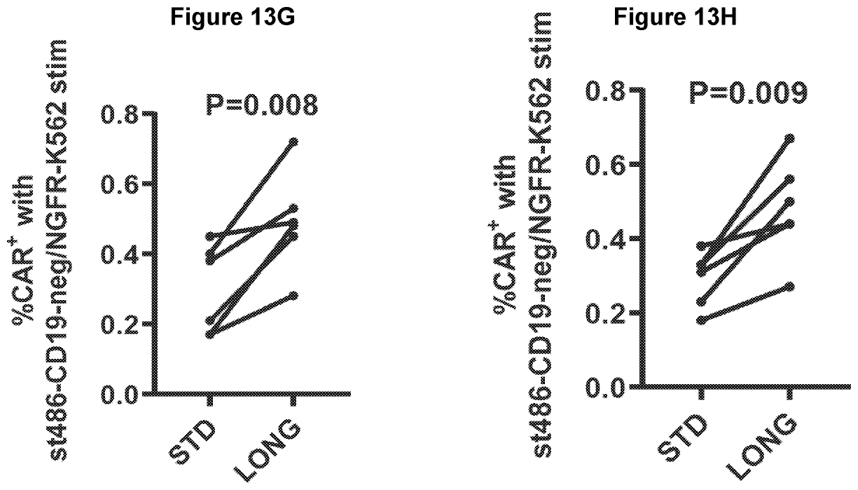

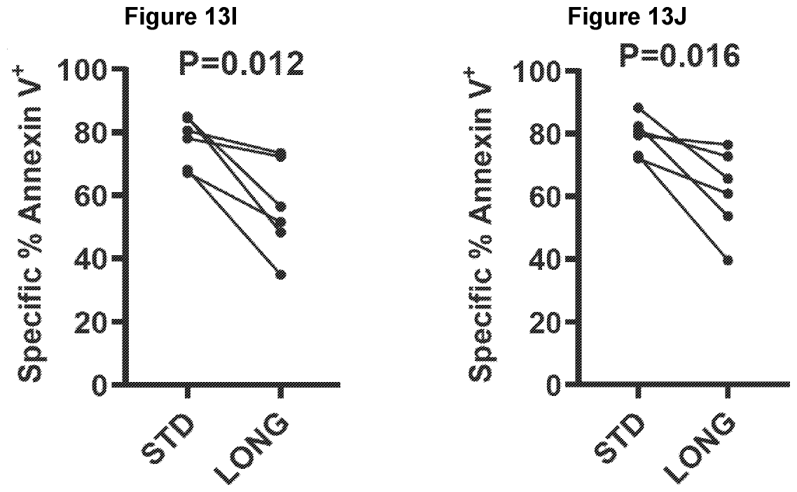

FIGS. 13A-13J show that lengthening the linker of the Hu20 scFv has a functional impact on CAR T cells.

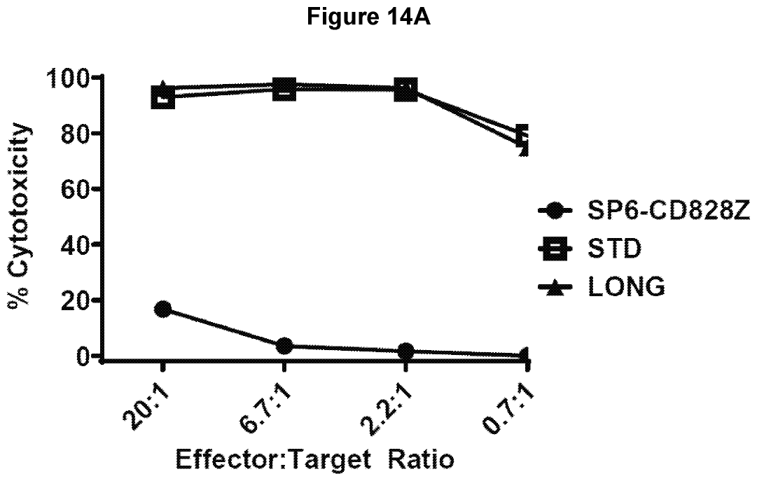

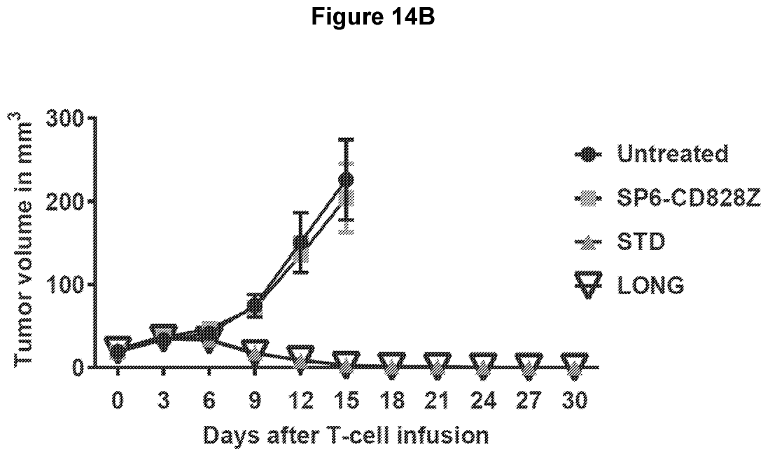

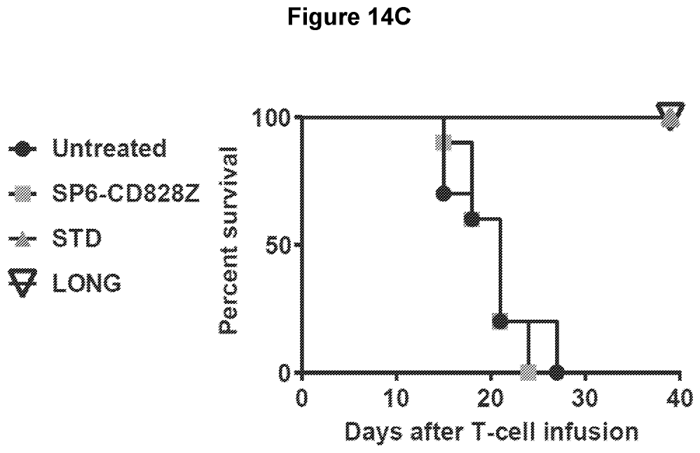

FIGS. 14A-14C show in vitro cytotoxicity and murine tumor reduction.

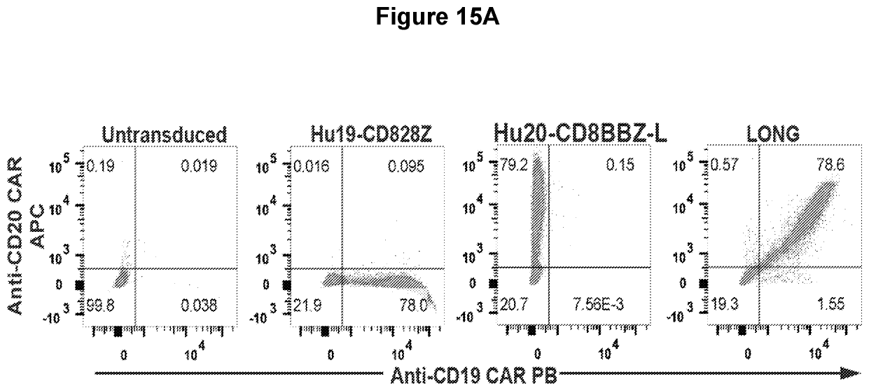

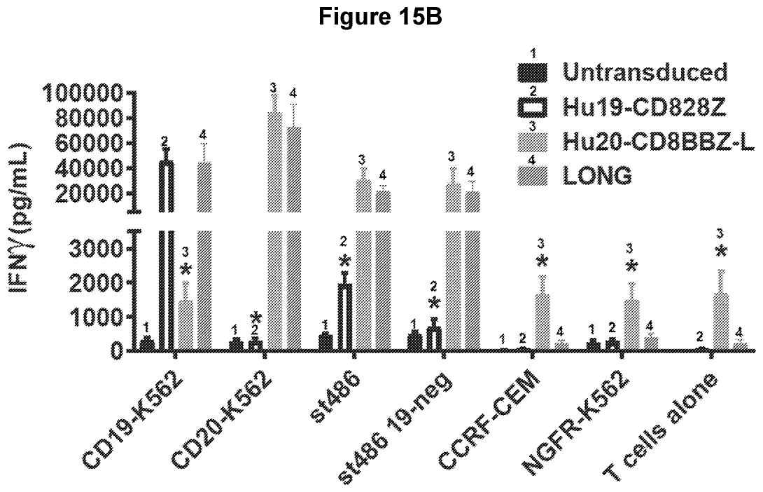

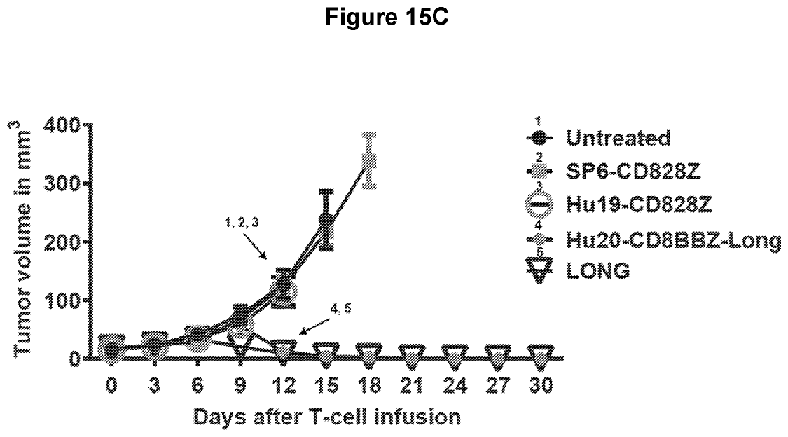

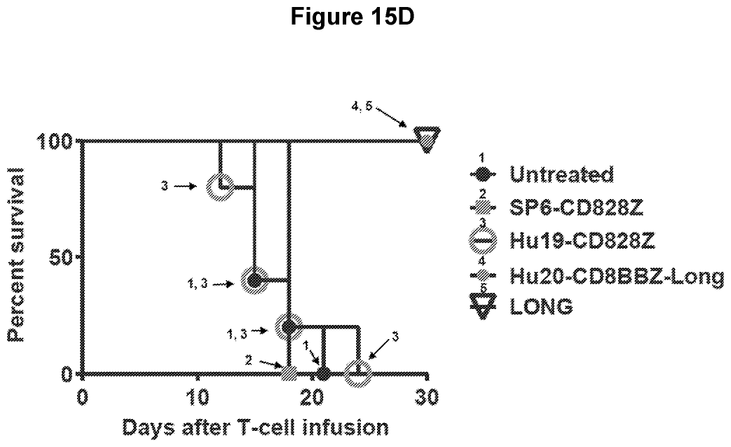

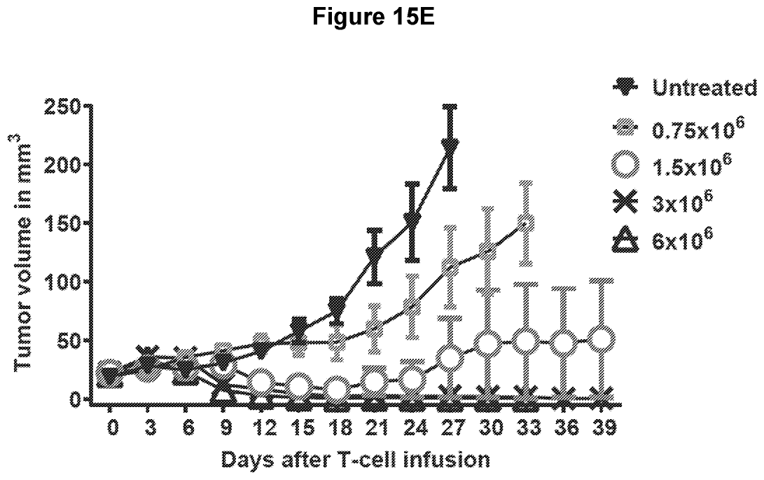

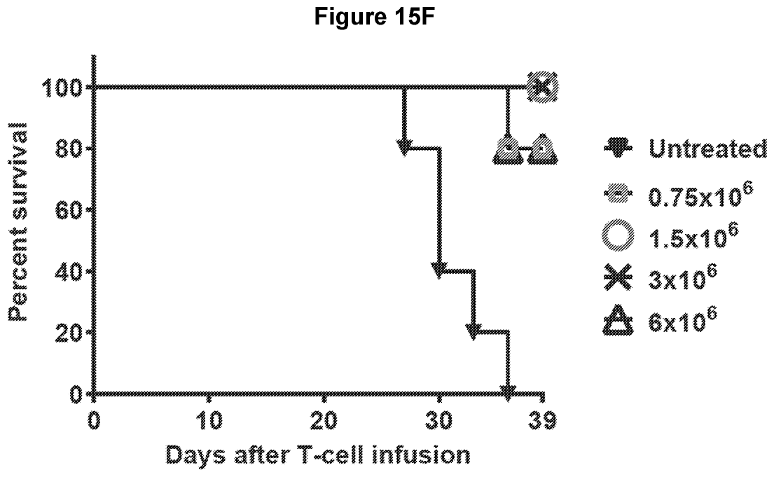

FIGS. 15A-15F further show antigen-specific CAR T-cell function.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid comprising a nucleotide sequence encoding a chimeric antigen receptor (CAR) construct comprising:
(a) a first CAR comprising
a first antigen binding domain,
a first transmembrane domain, and
a first intracellular T cell signaling domain;
(b) a second CAR comprising
a second antigen binding domain,
a second transmembrane domain, and
a second intracellular T cell signaling domain; and
(c) a cleavage sequence,
wherein the cleavage sequence is positioned between the first and second CARs; and wherein the nucleic acid has been designed to reduce retroviral recombination.

2. The nucleic acid of claim 1, wherein the nucleic acid sequence identity between the first and second CARs is no more than 90%.

3. The nucleic acid of claim 1, wherein the nucleic acid, when expressed in a host cell, exhibits greater expression compared to a nucleic acid that encodes the same amino acid sequence but that has not been designed to reduce retroviral recombination.

4. A nucleic acid comprising a nucleotide sequence encoding a chimeric antigen receptor (CAR) construct comprising:
(a) a first CAR comprising
a first antigen binding domain,
a first transmembrane domain, and
a first intracellular T cell signaling domain;
(b) a second CAR comprising
a second antigen binding domain,
a second transmembrane domain, and
a second intracellular T cell signaling domain; and
(c) a cleavage sequence,
wherein the cleavage sequence is positioned between the first and second CARs; and
(d) wherein the first or second antigen binding domain comprises a linker of SEQ ID NO: 41.

5. The nucleic acid of claim 1, wherein the first antigen binding domain of the first CAR has antigenic specificity for CD19, and wherein the second antigen binding domain of the second CAR has antigenic specificity for CD20.

6. The nucleic acid of claim 1, wherein the cleavage sequence comprises any one of the following: porcine teschovirus-1 2A (P2A) amino acid sequence, equine rhinitis A virus (E2A) amino acid sequence, thosea asigna virus 2A (T2A) amino acid sequence, foot-and-mouth disease virus (F2A) amino acid sequence, or a furin-cleavable amino acid sequence, modified versions of any of the foregoing, or any combination of the foregoing.

7. The nucleic acid of claim 1, wherein the cleavage sequence comprises a foot-and-mouth disease virus (F2A) amino acid sequence.

8. The nucleic acid of claim 1, wherein the cleavage sequence comprises an amino acid sequence comprising SEQ ID NO: 37.

9. The nucleic acid of claim 1, wherein the first antigen binding domain comprises the six CDRs of Hul9 or 47G4.

10. The nucleic acid of claim 1, wherein the first antigen binding domain comprises single-chain variable fragment 47G4.

11. The nucleic acid of claim 1, wherein the second antigen binding domain comprises the six CDRs of 11B8, C2B8, 2.1.2, 8G6, or GA101.

12. The nucleic acid of claim 1, wherein the second antigen binding domain comprises an antigen binding domain of antibody C2B, 11B8, 8G6, 2.1.2, or GA101.

13. The nucleic acid of claim 1, wherein one or both of the first and second transmembrane domain(s) comprises a CD8 transmembrane domain and hinge domain.

14. The nucleic acid of claim 13, wherein one or both of the first and second CARs comprises the nucleic acid sequence of SEQ ID NO: 57 or 65.

15. The nucleic acid of claim 1, wherein one or both of the first and second intracellular T cell signaling domain(s) comprises any one of the following: a human CD28 protein, a human CD3-zeta protein, a human FcRy protein, a CD27 protein, an OX40 protein, a human 4-1BB protein, a human inducible T-cell costimulatory protein (ICOS), modified versions of any of the foregoing, or any combination of the foregoing.

16. The nucleic acid of claim 1, wherein one or both of the first and second intracellular T cell signaling domain(s) comprises a CD28 intracellular T cell signaling sequence or a 41BB sequence.

17. The nucleic acid of claim 16, wherein one or both of the first and second intracellular T cell signaling domain(s) comprises a CD28 intracellular T cell signaling sequence comprising the nucleic acid sequence of SEQ ID NO: 58 or 69; or wherein one or both of the first and second intracellular T cell signaling domain(s) comprises a 4-1BB intracellular T cell signaling sequence comprising the nucleic acid sequence of SEQ ID NO: 66.

18. The nucleic acid of claim 1, wherein one or both of the first and second intracellular T cell signaling domain(s) comprises a CD3 zeta (ζ) intracellular T cell signaling sequence.

19. The nucleic acid of claim 18, wherein the CD3ζ intracellular T cell signaling sequence comprises the nucleic acid sequence of SEQ ID NO: 59 or 67.

20. The nucleic acid of claim 1, wherein the CAR construct comprises a CD8 leader domain.

21. The nucleic acid of claim 20, wherein the CD8 leader domain sequence comprises the nucleic acid sequence of SEQ ID NO: 53.

22. The nucleic acid of claim 1, wherein the CAR construct comprises exactly two CARs being the first and second CARs, respectively.

23. The nucleic acid of claim 1, comprising the nucleic acid sequence of one or more of SEQ ID NOs: 42-45.

24. A nucleic acid comprising the nucleic acid sequence of one or more of SEQ ID NOs: 48-52.

25. A chimeric antigen receptor (CAR) comprising the amino acid sequence of any one of SEQ ID NOs: 71-79.

26. The CAR of claim 25, wherein the CAR comprises the amino acid sequence of SEQ ID NO: 72.

27. A recombinant expression vector comprising the nucleic acid of claim 1.

28. The recombinant expression vector of claim 27, wherein the vector is a gamma-retrovirus, lentivirus, or transposon vector.

29. An isolated host cell comprising the recombinant expression vector of claim 27.

30. The isolated host cell of claim 29, wherein the cell is a T cell, a macrophage, or a NK cell.

31. A population of cells comprising at least one host cell of claim 29.

32. A pharmaceutical composition comprising the nucleic acid of claim 1, or one or more of SEQ ID NOs: 48-52, (b) a CAR comprising the amino acid sequence of any one of SEQ ID NOs: 71-79, (c) a recombinant expression vector comprising the amino acid sequence of (a), (d) a host cell comprising the recombinant expression vector of (c), or (e) a population of cells comprising at least one host cell of (d), and a pharmaceutically acceptable carrier.

33. A method of detecting the presence of cancer in a mammal, comprising:
(a) contacting a sample comprising one or more cells from the mammal with (a)the nucleic acid of claim 1, or one or more of SEQ ID NOs: 48-52, (b) a CAR comprising the amino acid sequence of any one of SEQ ID NOs: 71-79, (c) a recombinant expression vector comprising the amino acid sequence of (a), (d) a host cell comprising the recombinant expression vector of (c), (e) a population of cells comprising at least one host cell of (d), or a pharmaceutical composition comprising one or more of (a) through (e) and a pharmaceutically acceptable carrier thereby forming a complex, and
(b) detecting the complex, wherein detection of the complex is indicative of the presence of cancer in the mammal.

34. A method of treatment or prevention of cancer in a mammal, comprising administering to the mammal (a) the nucleic acid of claim 1, or one or more of SEQ ID NOs: 48-52, (b) a CAR comprising the amino acid sequence of any one of SEQ ID NOs: 71-79, (c) a recombinant expression vector comprising the amino acid sequence of (a), (d) a host cell comprising the recombinant expression vector of (c), (e) a population of cells comprising at least one host cell of (d), or a pharmaceutical composition comprising one or more of (a) through (e) and a pharmaceutically acceptable carrier, whereby cancer is treated or prevented in the mammal.

35. The method of claim 34, wherein the administration comprises (d) or (e) or a pharmaceutical composition comprising (d) or (e) and a pharmaceutically acceptable carrier.

36. The method of claim 35, wherein the host cell or population of cells is autologous in relation to the mammal.

37. The method of claim 35, wherein the host cell or population of cells is allogeneic in relation to the mammal.

38. The method of claim 34,
wherein the cancer is a hematological malignancy.

39. A method of making a chimeric antigen receptor (CAR) construct, the method comprising:
(i) designing a nucleic acid comprising a nucleotide sequence encoding a CAR construct comprising
(a) a first CAR comprising
a first antigen binding domain,
a first transmembrane domain, and
a first intracellular T cell signaling domain;
(b) a second CAR comprising
a second antigen binding domain,
a second transmembrane domain, and
a second intracellular T cell signaling domain; and
(c) a cleavage sequence,
wherein the cleavage sequence is positioned between the first and second CARs;
(ii) designing the nucleic acid to reduce retroviral recombination; and
(iii) preparing the nucleic acid of (ii).

40. The method of claim 39, wherein the sequence identity between the first and second CARs is no more than 90%.

41. The method of claim 39, wherein the nucleic acid, when expressed in a host cell, exhibits greater expression compared to a nucleic acid that encodes the same amino acid sequence but that has not been designed to reduce retroviral recombination.

42. The method of claim 39, wherein the method further comprises expressing the CAR construct in a host cell.

43. The method of claim 39, wherein the nucleic acid sequence comprises of any one of SEQ ID NOs: -48-52.


SEQUENCE LISTING (80 sequences)

SEQ ID NO: 1 - DNA, 249 bp, Homo sapiens
ttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctggtcatcaccctgtactgcaaccaccggaac

SEQ ID NO: 2 - DNA, 249 bp, Homo sapiens
ttcgtgcctgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatttgggcccctctggctggaacttgtggcgtgctgctgctgtctctcgtgatcacactgtattgcaatcataggaac

SEQ ID NO: 3 - DNA, 249 bp, Homo sapiens
ttcgtgccagtgtttctacctgccaagccgaccaccacgcctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctcagcctggtcatcaccctgtactgtaaccaccggaac

SEQ ID NO: 4 - DNA, 249 bp, Homo sapiens
ttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaac

SEQ ID NO: 5 - protein, 119 aa, Artificial Sequence
  Synthetic
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 6 - protein, 108 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 7 - protein, 121 aa, Mus musculus [sequence omitted]

SEQ ID NO: 8 - protein, 106 aa, Mus musculus [sequence omitted]

SEQ ID NO: 9 - protein, 125 aa, Homo sapiens
EVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 10 - protein, 15 aa, Artificial Sequence
  Synthetic
GGGGSGGGGSGGGGS

SEQ ID NO: 11 - protein, 123 aa, Homo sapiens
EVQLAESGGDLVQSGRSLRLSCAASGITFHDYAMHWVRQPPGKGLEWVSGISWNSDYIGYADSVKGRFTISRDNAKKSLYLQMNSLRPDDTALYYCVKDFHYGSGSNYGMDVWGQGTTVTVSS

SEQ ID NO: 12 - protein, 107 aa, Homo sapiens
EIVMTQSPATLSMSPGERATLSCRASQSVSRNLAWYQQKVGQAPRLLISGASTRATGIPARFSGSGSGTEFTLTINSLQSEDFAVYYCQQSNDWPLTFGQGTRLEIK

SEQ ID NO: 13 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 14 - protein, 112 aa, Homo sapiens
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 15 - protein, 119 aa, Artificial Sequence
  Synthetic
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 112 aa, Artificial Sequence
  Synthetic
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 17 - protein, 11 aa, Homo sapiens
RASQSVSSYLA

SEQ ID NO: 18 - protein, 7 aa, Homo sapiens
DASNRAT

SEQ ID NO: 19 - protein, 9 aa, Homo sapiens
QQRSDWPLT

SEQ ID NO: 20 - protein, 6 aa, Homo sapiens
SYHAMH

SEQ ID NO: 21 - protein, 16 aa, Homo sapiens
IIGTGGVTYYADSVKG

SEQ ID NO: 22 - protein, 17 aa, Homo sapiens
DYYGAGSFYDGLYGMDV

SEQ ID NO: 23 - protein, 16 aa, Artificial Sequence
  Synthetic
RSSKSLLHSNGITYLY

SEQ ID NO: 24 - protein, 7 aa, Artificial Sequence
  Synthetic
QMSNLVS

SEQ ID NO: 25 - protein, 9 aa, Artificial Sequence
  Synthetic
AQNLELPYT

SEQ ID NO: 26 - protein, 6 aa, Artificial Sequence
  Synthetic
GYAFSY

SEQ ID NO: 27 - protein, 8 aa, Artificial Sequence
  Synthetic
FPGDGDTD

SEQ ID NO: 28 - protein, 10 aa, Artificial Sequence
  Synthetic
NVFDGYWLVY

SEQ ID NO: 29 - protein, 18 aa, Artificial Sequence
  Synthetic
GSTSGSGKPGSGEGSTKG

SEQ ID NO: 30 - protein, 21 aa, Homo sapiens
MALPVTALLLPLALLLHAARP

SEQ ID NO: 31 - protein, 83 aa, Homo sapiens
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 32 - protein, 41 aa, Homo sapiens
RSKRSRLLHSDYMNMTPRRPGPTRKHYQPYAPPRDFAAYRS

SEQ ID NO: 33 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 42 aa, Homo sapiens
KRGRKKLLYIFKQPFMRPVQTTQEEDGCSCRFPEEEEGGCEL

SEQ ID NO: 35 - protein, 9 aa, Artificial Sequence
  Synthetic
  MISC_FEATURE (5)..(5): Xaa can be any naturally occurring amino acid
GDVEXNPGP

SEQ ID NO: 36 - protein, 4 aa, Artificial Sequence
  Synthetic
SGSG

SEQ ID NO: 37 - protein, 32 aa, Artificial Sequence
  Synthetic
RAKRSGSGAPVKQTLNFDLLKLAGDVESNPGP

SEQ ID NO: 38 - protein, 4 aa, Artificial Sequence
  Synthetic
RAKR

SEQ ID NO: 39 - protein, 6 aa, Artificial Sequence
  Synthetic
SGSGAP

SEQ ID NO: 40 - protein, 22 aa, Artificial Sequence
  Synthetic
VKQTLNFDLLKLAGDVESNPGP

SEQ ID NO: 41 - protein, 20 aa, Artificial Sequence
  Synthetic
GGGGSGGGGSGGGGSGGGGS

SEQ ID NO: 42 - DNA, 3129 bp, Artificial Sequence
  Synthetic
atggccctgcctgtgacagctctgctgctgcccctggccctgctgctgcatgccgccagacctgagatcgtgctgacccagtctcccggtaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggtaccgacttcaccctgaccatcagcagactggaacccgaggacttcgccgtgtattactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtcaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgccagtgtttctacctgccaagccgaccaccacgcctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctcagcctggtcatcaccctgtactgtaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaggagggcctgtataatgagctgcagaaagacaagatggccgaggcctacagcgagatcggcatgaaaggcgagagaagaagaggcaagggccacgatggactgtaccagggactgagcacagccaccaaggatacctacgatgccctgcacatgcaggcccttccacctagaagggccaagagatctggatctggcgcccctgtgaagcagaccctgaatttcgacctgctgaagctggccggcgacgtggaatctaatcctggacctatgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaagcgaggccggaagaagctgctgtacatcttcaagcagcctttcatgcggcccgtgcagaccacacaagaggaagatggctgtagctgcagattccccgaggaagaagaaggcggctgcgagctgagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 43 - DNA, 3144 bp, Artificial Sequence
  Synthetic
atggccctgcctgtgacagctctgctgctgcccctggccctgctgctgcatgccgccagacctgagatcgtgctgacccagtctcccggtaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggtaccgacttcaccctgaccatcagcagactggaacccgaggacttcgccgtgtattactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtcaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgccagtgtttctacctgccaagccgaccaccacgcctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctcagcctggtcatcaccctgtactgtaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaggagggcctgtataatgagctgcagaaagacaagatggccgaggcctacagcgagatcggcatgaaaggcgagagaagaagaggcaagggccacgatggactgtaccagggactgagcacagccaccaaggatacctacgatgccctgcacatgcaggcccttccacctagaagggccaagagatctggatctggcgcccctgtgaagcagaccctgaatttcgacctgctgaagctggccggcgacgtggaatctaatcctggacctatgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggaggaggcgggagtggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaagcgaggccggaagaagctgctgtacatcttcaagcagcctttcatgcggcccgtgcagaccacacaagaggaagatggctgtagctgcagattccccgaggaagaagaaggcggctgcgagctgagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 44 - DNA, 3141 bp, Artificial Sequence
  Synthetic
atggccctgcctgtgacagctctgctgctgcccctggccctgctgctgcatgccgccagacctgagatcgtgctgacccagtctcccggtaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggtaccgacttcaccctgaccatcagcagactggaacccgaggacttcgccgtgtattactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtcaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgccagtgtttctacctgccaagccgaccaccacgcctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctcagcctggtcatcaccctgtactgtaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaggagggcctgtataatgagctgcagaaagacaagatggccgaggcctacagcgagatcggcatgaaaggcgagagaagaagaggcaagggccacgatggactgtaccagggactgagcacagccaccaaggatacctacgatgccctgcacatgcaggcccttccacctagaagggccaagagatctggatctggcgcccctgtgaagcagaccctgaatttcgacctgctgaagctggccggcgacgtggaatctaatcctggacctatgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggaggaggcgggagtggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaggagtaagaggagcaggctcctgcatagtgattatatgaatatgactccccgccgccccgggcccacccgcaagcattatcagccctatgccccaccacgcgacttcgcagcctaccgctccagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 45 - DNA, 3126 bp, Artificial Sequence
  Synthetic
atggccctgcctgtgacagctctgctgctgcccctggccctgctgctgcatgccgccagacctgagatcgtgctgacccagtctcccggtaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggtaccgacttcaccctgaccatcagcagactggaacccgaggacttcgccgtgtattactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtcaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgccagtgtttctacctgccaagccgaccaccacgcctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctcagcctggtcatcaccctgtactgtaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaggagggcctgtataatgagctgcagaaagacaagatggccgaggcctacagcgagatcggcatgaaaggcgagagaagaagaggcaagggccacgatggactgtaccagggactgagcacagccaccaaggatacctacgatgccctgcacatgcaggcccttccacctagaagggccaagagatctggatctggcgcccctgtgaagcagaccctgaatttcgacctgctgaagctggccggcgacgtggaatctaatcctggacctatgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaggagtaagaggagcaggctcctgcatagtgattatatgaatatgactccccgccgccccgggcccacccgcaagcattatcagccctatgccccaccacgcgacttcgcagcctaccgctccagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 46 - protein, 1041 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMALPVTALLLPLALLLHAARPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 3126 bp, Artificial Sequence
  Synthetic
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctggtcatcaccctgtactgcaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggcatgaagggcgagagaagaagaggcaagggccacgatggactgtaccagggactgagcacagccaccaaggatacctacgatgccctgcacatgcaggcccttccacctagaagggccaagagatctggatctggcgcccctgtgaagcagaccctgaatttcgacctgctgaagctggccggcgacgtggaatctaatcctggacctatggctctgcccgtgacagctttgctgctgcctctggctctgctgctgcatgccgctagacccgatatcgtgatgacccagacacctcacagcagccctgttacactgggacagcctgccagcatctcctgtagaagcagccagagcctggtgtccagagatggcaatacctacctgagctggctgcagcagaggcctggacaacctcctagactgctgatctacaagatcagcaaccggttcagcggcgtgcccaatagattttctggaagcggagccggcaccgacttcaccctgaagatttctagagtgaaggccgaggacgtgggcgtgtactactgtatgcaggccacacagttccctctgacctttggccagggcaccagactggaaatcaaaggtggcggaggttctggcggcggaggatcaggcggaggtggaagtgaagtgcagctggttcagtctggcgccgaagtgaagaagcctggcgagtctctgaagatcagctgcaaaggcagcggctacagcttcaccagctattggatcggctgggtccgacagatgcctggcaaaggactggaatggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggacagggcacaacagtgaccgtgtctagcttcgtgcctgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatttgggcccctctggctggaacttgtggcgtgctgctgctgtctctcgtgatcacactgtattgcaatcataggaacaagcgaggccggaagaagctgctgtacatcttcaagcagcctttcatgcggcccgtgcagaccacacaagaggaagatggctgtagctgcagattccccgaggaagaagaaggcggctgcgagctgagagtgaaattctctagaagcgccgacgcacccgcataccagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 48 - DNA, 1509 bp, Artificial Sequence
  Synthetic
atggccctgcctgtgacagctctgctgctgcccctggccctgctgctgcatgccgccagacctgagatcgtgctgacccagtctcccggtaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggtaccgacttcaccctgaccatcagcagactggaacccgaggacttcgccgtgtattactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtcaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgccagtgtttctacctgccaagccgaccaccacgcctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctcagcctggtcatcaccctgtactgtaaccaccggaacagaagcaagcggagcagactgctgcacagcgactacatgaacatgacccctagacggcccggacctaccagaaagcactaccagccttacgctcctcctcgggactttgccgcctatcggagcagagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaggagggcctgtataatgagctgcagaaagacaagatggccgaggcctacagcgagatcggcatgaaaggcgagagaagaagaggcaagggccacgatggactgtaccagggactgagcacagccaccaaggatacctacgatgccctgcacatgcaggcccttccacctagataa

SEQ ID NO: 49 - DNA, 1527 bp, Artificial Sequence
  Synthetic
atgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaagcgaggccggaagaagctgctgtacatcttcaagcagcctttcatgcggcccgtgcagaccacacaagaggaagatggctgtagctgcagattccccgaggaagaagaaggcggctgcgagctgagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 50 - DNA, 1524 bp, Artificial Sequence
  Synthetic
atgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaggagtaagaggagcaggctcctgcatagtgattatatgaatatgactccccgccgccccgggcccacccgcaagcattatcagccctatgccccaccacgcgacttcgcagcctaccgctccagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 51 - DNA, 1542 bp, Artificial Sequence
  Synthetic
atgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggaggaggcgggagtggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaagcgaggccggaagaagctgctgtacatcttcaagcagcctttcatgcggcccgtgcagaccacacaagaggaagatggctgtagctgcagattccccgaggaagaagaaggcggctgcgagctgagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 52 - DNA, 1539 bp, Artificial Sequence
  Synthetic
atgcttctcctggtgacaagccttctgctctgtgagttaccacacccagcattcctcctgatcccagatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaaggaggaggcgggagtggtggcggaggttccggcggcggaggatcaggcggaggtggaagtgaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagcttcgttccggtttttctgccggcaaagcctacaactacccccgcaccccggcccccaactcccgctccaacgatcgcatcacaaccactttcactccgaccagaggcttgcagaccggctgcgggaggcgcggtacacacgcgggggctcgattttgcttgcgatatttacatttgggctcctcttgccggtacatgcggtgtcttgctcctgtccctcgtcattactctctattgcaaccataggaacaggagtaagaggagcaggctcctgcatagtgattatatgaatatgactccccgccgccccgggcccacccgcaagcattatcagccctatgccccaccacgcgacttcgcagcctaccgctccagggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgctaa

SEQ ID NO: 53 - DNA, 63 bp, Artificial Sequence
  Synthetic
atggccctgcctgtgacagctctgctgctgcccctggccctgctgctgcatgccgccagacct

SEQ ID NO: 54 - DNA, 324 bp, Artificial Sequence
  Synthetic
gagatcgtgctgacccagtctcccggtaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggtaccgacttcaccctgaccatcagcagactggaacccgaggacttcgccgtgtattactgccagcagtacggcagcagccggttcaccttcggccctggcaccaaggtggacatcaag

SEQ ID NO: 55 - DNA, 54 bp, Artificial Sequence
  Synthetic
ggcagcacctccggcagcggcaagcctggctctggcgagggctctaccaagggc

SEQ ID NO: 56 - DNA, 357 bp, Artificial Sequence
  Synthetic
caggtgcagctggtgcagtctggcgccgaagtcaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagc

SEQ ID NO: 57 - DNA, 249 bp, Artificial Sequence; identical to SEQ ID NO: 3
  Synthetic

SEQ ID NO: 58 - DNA, 123 bp, Artificial Sequence
  Synthetic
agaagcaagcggagcagactgctgcacagcgactacatgaacatgacccctagacggcccggacctaccagaaagcactaccagccttacgctcctcctcgggactttgccgcctatcggagc

SEQ ID NO: 59 - DNA, 336 bp, Artificial Sequence
  Synthetic
agagtgaagttcagcagatcagccgatgctcctgcctaccagcagggccagaatcagctgtacaacgagctgaacctggggagaagagaagagtacgacgtgctggataagcggagaggcagagatcctgagatgggcggcaagcccagacggaagaatcctcaggagggcctgtataatgagctgcagaaagacaagatggccgaggcctacagcgagatcggcatgaaaggcgagagaagaagaggcaagggccacgatggactgtaccagggactgagcacagccaccaaggatacctacgatgccctgcacatgcaggcccttccacctaga

SEQ ID NO: 60 - DNA, 96 bp, Artificial Sequence
  Synthetic
agggccaagagatctggatctggcgcccctgtgaagcagaccctgaatttcgacctgctgaagctggccggcgacgtggaatctaatcctggacct

SEQ ID NO: 61 - DNA, 66 bp, Artificial Sequence
  Synthetic
atgcttctcctggtgacaagccttctgctctgtgagttaccacacccagcattcctcctgatccca

SEQ ID NO: 62 - DNA, 336 bp, Artificial Sequence
  Synthetic
gatatcgtgatgacacagacacctcacagcagccctgttacactgggacagcctgccagcatctcctgtagaagctcccagagcctggtgtccagagatggcaatacctacctgagctggctgcagcagaggcctggacaacctcctaggctgctgatttacaagatcagcaaccggttcagcggcgtgcccaatagattttctggaagcggagccggcacagactttaccctgaagatttctagagtgaaggccgaggacgtgggcgtgtactactgtatgcaggccacacagttccctctgacctttggccagggcaccagactggaaatcaaa

SEQ ID NO: 63 - DNA, 45 bp, Artificial Sequence
  Synthetic
ggtggcggaggttccggcggcggaggatcaggcggaggtggaagt

SEQ ID NO: 64 - DNA, 366 bp, Artificial Sequence
  Synthetic
gaagtccagctcgttcagtccggagccgaggtgaagaagcctggcgagtctctgaagatcagctgcaaaggcagcggctacagcttcaccagctattggatcggctgggtccgacagatgcctggcaaaggactggagtggatgggcatcatctaccccggcgacagcgataccagatacagccctagctttcagggccaagtgaccatcagcgccgacaagagcatcagcacagcctacctgcagtggtctagcctgaaggccagcgacaccgccatgtactattgtgccagacagggcgacttttggagcggctatggtggcatggatgtgtggggccagggcacaacagtgaccgtgtctagc

SEQ ID NO: 65 - DNA, 249 bp, Artificial Sequence; identical to SEQ ID NO: 4
  Synthetic

SEQ ID NO: 66 - DNA, 126 bp, Artificial Sequence
  Synthetic
aagcgaggccggaagaagctgctgtacatcttcaagcagcctttcatgcggcccgtgcagaccacacaagaggaagatggctgtagctgcagattccccgaggaagaagaaggcggctgcgagctg

SEQ ID NO: 67 - DNA, 336 bp, Artificial Sequence
  Synthetic
agggtgaaattctctagaagcgccgacgcacccgcatatcagcaaggacaaaaccagctctataacgaactcaacctcggcagacgcgaggaatatgatgtgctggacaagaggcggggacgcgatccagaaatgggaggaaagcctcggagaaagaacccacaagagggactttacaacgaactccaaaaggataagatggcagaagcctattccgagattggaatgaagggcgaacgtcggagaggaaagggacacgacggcctttatcagggcctgtccaccgccacaaaagatacgtatgacgctctccacatgcaagcgttgcccccccgc

SEQ ID NO: 68 - DNA, 60 bp, Artificial Sequence
  Synthetic
ggaggaggcgggagtggtggcggaggttccggcggcggaggatcaggcggaggtggaagt

SEQ ID NO: 69 - DNA, 123 bp, Artificial Sequence
  Synthetic
aggagtaagaggagcaggctcctgcatagtgattatatgaatatgactccccgccgccccgggcccacccgcaagcattatcagccctatgccccaccacgcgacttcgcagcctaccgctcc

SEQ ID NO: 70 - protein, 5 aa, Artificial Sequence
  Synthetic
GGGGS

SEQ ID NO: 71 - protein, 1042 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 1047 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 1046 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 1041 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGAPVKQTLNFDLLKLAGDVESNPGPMLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 502 aa, Artificial Sequence
  Synthetic
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 508 aa, Artificial Sequence
  Synthetic
MLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 507 aa, Artificial Sequence
  Synthetic
MLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 513 aa, Artificial Sequence
  Synthetic
MLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 512 aa, Artificial Sequence
  Synthetic
MLLLVTSLLLCELPHPAFLLIPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 22 aa, Artificial Sequence
  Synthetic
MLLLVTSLLLCELPHPAFLLIP